(19) United States
(12) Patent Application Publication
(10) Pub. No.: US 2020/0185054 A1
(43) Pub. Date: Jun. 11, 2020

(54) DEVICE AND METHOD OF IDENTIFYING AND EVALUATING A TUMOR PROGRESSION

(71) Applicant: TURING AI INSTITUTE (NANJING) CO., LTD., Nanjing (CN)

(72) Inventors: Jianyang ZENG, Nanjing (CN); Bin ZHOU, Nanjing (CN)

(73) Assignee: TURING AI INSTITUTE (NANJING) CO., LTD., Nanjing (CN)

(21) Appl. No.: 16/725,147

(22) Filed: Dec. 23, 2019

Related U.S. Application Data
(63) Continuation of application No. PCT/CN2019/082574, filed on Apr. 12, 2019.

(30) Foreign Application Priority Data
Apr. 16, 2018 (CN) 201810337789.8

(51) Int. Cl. G16B 20/20; G16B 20/10; G16H 50/20

(52) U.S. Cl. CPC G16B 20/20; G16H 50/20; G16B 20/10

(57) ABSTRACT

The present application relates to a device and method of identifying and evaluating a tumor progression. The device or method can comprise: 1) a module or step capable of providing a clinical feature of a patient with the tumor; 2) a module or step capable of providing at least one biological indicator derived from the patient; 3) a module or step capable of determining a correlation between the at least one biological indicator of the individual patient and the clinical feature of the same patient; and 4) a module or step capable of evaluating a tumor progression or identifying an evaluation indicator of the correlation. The device or method of the present application are capable of providing guidance for study in potential molecular mechanisms of the tumor progression and providing the therapeutic strategy against the tumor progression.

### TECHNICAL FIELD

The present application relates to the detection and treatment of diseases, especially to a device and a method of identifying a biological indicator capable of evaluating a tumor progression, and to a device and a method of determining a tumor progression.

### INVENTION BACKGROUND

It is one of the most important problems in oncology to reveal the potential molecular mechanisms of tumorgenesis. High-throughout DNA-decoding technology can offer the genomic features of a patient with gene expression disorders. For example, it has been found that copy number variation (CNV) can function as an important indicator of cancers like colorectal cancers (see, Zhao S, et al., Proc Natl Acad Sci U S A 2013, 110 (8): 2916-2921). DNA methylation is an important epigenetic mechanism. In urinary bladder carcinoma cells, abnormal levels of DNA methylation have been shown to be associated with dysfunction of certain genes, and thus associated with the occurrence of urinary bladder carcinoma (see, Rose M, et al., Carcinogenesis 2014, 35 (3): 727-736). Somatic mutations are often considered as another cause of the bladder carcinoma progression (see, Soung Y H, et al., Oncogene 2003, 22 (39): 8048-8052). Also, abnormal expressions of microRNA may lead to disorder of intracellular regulatory network in bladder carcinoma cells (see, Jin Y, et al., Tumour Biol 2015, 36 (5): 3791-3797).

However, the occurrence and progression of cancers are often a multi-step and highly dynamic process which involves the activity level variations of a plurality of molecules in cells. Thus, it is generally difficult to evaluate the progression or prognosis of cancers by a single indicator. Moreover, there is an absence of reliable biological indicator correlated with the clinical feature (e.g., the progression of diseases) in the correlative field. Correspondingly, there is an urgent need of identifying potential biological indicators capable of revealing the cancer progression; evaluating the important biological indicators associated with the cancer progression from various viewpoints, such as, gene expression levels, copy number variations, DNA methylations, somatic mutations, and microRNA regulations; and studying how to evaluate the progression and/or prognosis of the cancer by comprehensive use of these indicators.

### SUMMARY OF THE INVENTION

The present application provides a device and method of identifying a biological indicator capable of evaluating a tumor progression, and said device and method can creatively compare and associate a clinical feature of a patient with tumor (such as, the tumor stage and/or the survival time of the patient) with at least one biological indicator of the patient (e.g., expression level of gene, copy number variation, DNA methylation, somatic mutations, microRNAs, and so on) to identify a biological indicator capable of evaluating the tumor progression. Furthermore, the present application further provides a device and a method of determining a tumor progression in a subject, and said device and method can creatively comprehensively utilize various biological indicators as identified and assign reasonable weights to the various indicators, and accordingly determine the circumstance of the tumor progression in the subject. Under certain circumstances, the device or method of the present application can further provide a suitable therapeutic regimen on the basis of the determined results.

In one aspect, the present application provides a device of identifying a biological indicator capable of evaluating a tumor progression comprising: 1) a clinical feature module capable of providing a clinical feature of a patient with the tumor, wherein the clinical feature comprises the tumor stage of patient and/or the survival time of the patient; 2) a biological indicator module capable of providing at least one biological indicator derived from the patient; 3) a correlation determination module capable of determining a correlation between the at least one biological indicator of the individual patient with the clinical feature of the corresponding patient; and 4) an identification module capable of identifying the biological indicator which is determined to be correlated with the clinical feature in the module 3) as being capable of evaluating the tumor progression.

In another aspect, the present application provides a device of identifying a biological indicator capable of evaluating a tumor progression comprising a computer for identifying the biological indicator, said computer being programmed to executing the steps of: 1) providing a clinical feature of a patient with the tumor, wherein the clinical feature comprises the tumor stage of patient and/or the survival time of the patient; 2) providing at least one biological indicator derived from the patient; 3) determining a correlation between the at least one biological indicator of the individual patient and the clinical feature of the corresponding patient; and 4) identifying the biological indicator which is determined to be correlated with the clinical feature in 3) as being capable of evaluating the tumor progression.

In another aspect, the present application provides a method of identifying a biological indicator capable of evaluating a progression of a tumor comprising: 1) providing a clinical feature of a patient with the tumor, wherein the clinical feature comprises the tumor stage of patient and/or the survival time of the patient; 2) providing at least one biological indicator derived from the patient; 3) determining a correlation between the at least one biological indicator of the individual patient and the clinical feature of the corresponding patient; and 4) identifying the biological indicator which is determined to be correlated with the clinical feature in 3) as being capable of evaluating the tumor progression.

In certain embodiments, the tumor comprises bladder cancer. In certain embodiments, bladder cancer comprises Bladder Urothelial Carcinoma (BLCA).

In certain embodiments, the tumor stage is selected from the group consisting of: Tumor Stage I, Tumor Stage II, Tumor Stage III, and Tumor Stage IV.

In certain embodiments, the at least one biological indicator comprises one or more classes of indicators selected from the group consisting of:

Class 1: an expression level of gene in the patient;

Class 2: a copy number variation of gene in the patient;

Class 3: a DNA methylation of gene in the patient;

Class 4: a somatic mutation of gene in the patient; and

Class 5: a microRNAs in the patient.

In certain embodiments, the at least one biological indicator comprises the expression level of gene in the patient, and determining a correlation between the expression level of gene and the clinical feature comprises: performing a single variable regression analysis in relation to the clinical feature by use of the expression level of gene as the single variable, and identifying the genes of which the p value is less than or equal to a first threshold and the FDR value is less than or equal to a second threshold in the regression analysis as being correlated with the clinical feature.

In certain embodiments, the at least one biological indicator comprises the expression level of gene in the patient, and determining a correlation between the expression level of gene and the clinical feature comprises performing a multiple-variable regression analysis against the clinical feature, and identifying the gene of which the FDR value is less than or equal to a third threshold in the regression analysis as being correlated with the clinical feature, and wherein and the multiple variable comprises the expression level of gene in the patient, the age of the patient, the gender of the patient, and/or the tumor stage of the patient.

In certain embodiments, the at least one biological indicator comprises the expression level of gene in the patient, and determining the correlation between the expression level of gene and the clinical feature further comprises: classifying the genes into protective effective genes and risk effective genes in accordance with the correlation coefficient values of the individual genes obtained in the multiple variable regression analysis, wherein the protective effective genes have negative correlation coefficient values, and the risk effective genes has positive correlation coefficient values.

In certain embodiments, the at least one biological indicator comprises the expression level of gene in the patient, and the determining the correlation between the expression level of gene and the clinical feature further comprises determining the expression level of gene in the patient in the individual tumor stage, determining accordingly the co-expression circumstances of genes which are specific for tumor staging, classifying the genes into two or more groups in accordance with the co-expression circumstances of the genes, and determining the correlation between the expression level of gene of each group and the clinical feature.

In certain embodiments, the method or device classify the genes into two or more groups in accordance with the co-expression circumstances of the genes by use of WGCNA algorithm.

In certain embodiments, the at least one biological indicator comprises the copy number variation of gene in the patient, and determining the correlation between the copy number variation of gene and the clinical feature comprises: comparing the copy number variation frequencies of gene in the patient in various tumor stages.

In certain embodiments, the at least one biological indicator comprises the DNA methylation of gene in the patient, and determining the correlation between the DNA methylation and the clinical feature comprises: performing a regression analysis in relation to the clinical feature by use of the degree of DNA methylation as the variable, and identifying the DNA methylation of which the p value is less than or equal to a fourth threshold in the regression analysis as being correlated with the clinical feature.

In certain embodiments, determining the correlation between the DNA methylation and the clinical feature in the device or method further comprises: determining a risk value of various DNA methylation sites which are determined to be correlated with the clinical feature, wherein the risk value is determined based on the correlation coefficient of the methylation site obtained in the regression analysis, as well as the methylation degree of the methylation site.

In certain embodiments, the at least one biological indicator comprises the somatic mutation of gene in the patient, and determining the correlation between the somatic mutation and the clinical feature comprises: determining the signal pathway of the gene containing the somatic mutation, and/or determining the correlation between the expression level of the gene containing the somatic mutation and the clinical feature.

In certain embodiments, the at least one biological indicator comprises the microRNA in the patient, and the determining the correlation between the microRNA and the clinical feature comprises: determining the correlation between the expression level of the gene regulated by the microRNA and the clinical feature, and determining the expression level of the microRNA in the patient and the expression level of the gene regulated by the microRNA.

In certain embodiments, the at least one biological indicator comprises two or more classes of the biological indicators, and the determining the correlation between the biological indicator and the clinical feature comprises determining a weights of various biological indicator affecting to the clinical feature.

In certain embodiments, the device or method determine the weight by means of ordered logistic regression analysis.

in certain embodiments, the at least one biological indicator comprises the expression level of gene in the patient, and determining a correlation between the expression level of gene and the clinical feature comprises: a) performing a single variable regression analysis to the clinical feature by use of the expression level of gene as the single variable, and identifying the gene of which the p value is less than or equal to a first threshold and the FDR value is less than or equal to a second threshold in the regression analysis as a first gene set correlated with the clinical feature.

In certain embodiments, determining the correlation between the expression level of gene and the clinical feature in the device or method further comprises: b) performing a multiple-variable regression analysis against to the clinical feature, and identifying the gene of which the FDR value is less than or equal to a third threshold in the regression analysis as a second gene set correlated with the clinical feature, and wherein the multiple variables comprise the expression level of the individual genes in the first gene set, the age of the patient, the gender of the patient, and the tumor stage of the patient.

In certain embodiments, determining the correlation between the expression level of gene and the clinical feature in the device or method further comprises: c) classifying the genes into protective effective genes and risk effective genes in accordance with the correlation coefficient values of the individual genes obtained in the multiple variable regression analysis, wherein the protective effective genes have negative correlation coefficient values, and the risk effective genes has positive correlation coefficient values.

In certain embodiments, determining the correlation between the expression level of gene and the clinical feature in the device or method further comprises: determining the expression level of the individual genes of the second gene set in various tumor stages, determining accordingly the co-expression circumstances of genes which are specific for tumor staging, classifying the genes of the second gene set into two or more groups in accordance with the co-expression circumstances of genes, and determining the correlation between the expression level of gene of each group and the clinical feature.

In certain embodiments, the device or method classify the genes in the second gene set into two or more groups in accordance with the co-expression circumstances of genes by use of WGCNA algorithm.

In certain embodiments, the at least one biological indicator further comprises the copy number variation of gene in the patient, and the determining the correlation between the copy number variation of gene and the clinical feature comprises: comparing the copy number variation frequencies of the genes of the second gene set in various tumor stages.

In certain embodiments, the at least one biological indicator further comprises the DNA methylation of gene in the patient, and determining the correlation between the DNA methylation and the clinical feature comprises: determining the DNA methylation sites of the genes of the second gene set and the DNA methylation degrees at the individual sites, performing a regression analysis against to the clinical feature by use of the DNA methylation degree as the variable, and identifying the DNA methylations of which the p value is less than or equal to a fourth threshold in the regression analysis as a first DNA methylation set associated with the clinical feature.

In certain embodiments, determining the correlation between the DNA methylation and the clinical feature in the device or method further comprises determining the risk values of various DNA methylation sites in the first DNA methylation set, wherein the risk values are determined based on the correlation coefficients of the methylation sites obtained in the regression analysis, as well as the methylation degrees of the methylation sites.

In certain embodiments, the at least one biological indicator further comprises the somatic mutation of gene in the patient, and determining the correlation between the somatic mutation and the clinical feature comprises: determining the somatic mutation contained in the gene in the second gene set, and determining the signal pathway of the gene containing the somatic mutation.

In certain embodiments, the at least one biological indicator comprises the microRNAs in the patient, and the determining the correlation between the microRNA and the clinical feature comprises: determining the microRNA regulating the gene of the second gene set, and determining the correlation between the expression level of the microRNA and the expression level of gene regulated by the microRNA, identifying the microRNA having a correlation higher than a fifth threshold as a first microRNA set correlated with the clinical feature.

In certain embodiments, determining the correlation between the biological indicator and the clinical feature in the device or method comprises: determining the weight of a biological indicator selected from group consisting of the expression level of the gene in the second gene set, the copy number variation of the gene in the second gene set, and the risk value of the DNA methylation site in the first DNA methylation set to the clinical feature by means of ordered logistic regression analysis, respectively.

In certain embodiments, the device or method determines the respective weights of the expression level of the protective effective genes and the risk effective genes of the second gene set, respectively.

In another aspect, the present application provides a computer readable storage media having a computer program stored, wherein the computer program allows the computer to execute the identifying method of the present application.

In another aspect, the present application provides a device of determining a tumor progression in a subject comprising: a) an analysis module capable of determining the expression levels of one or more genes as shown in Table 1 in the subject or a biological sample derived from the subject; and b) a determination module capable of determining the tumor progression in the subject in accordance with the expression level as measured in a).

In another aspect, the present application provides a device of determining a tumor progression in a subject comprising a computer for determining a tumor progression in a subject, said computer being programmed to executing the steps of: a) determining the expression levels of one or more genes as shown in Table 1 in the subject or a biological sample derived from the subject; and b) determining the tumor progression in the subject in accordance with the expression level as measured in a).

In another aspect, the present application provides a method of determining a tumor progression in a subject comprising: a) determining the expression levels of one or more genes as shown in Table 1 in the subject or a biological sample derived from the subject; and b) determining the tumor progression in the subject in accordance with the expression level as measured in a).

In certain embodiments, the tumor progression comprises the stages of the tumor and/or the survival rate of the subject.

In certain embodiments, the stage of the tumor is selected from the group consisting of: Tumor Stage I, Tumor Stage II, Tumor Stage III, and Tumor Stage IV.

In certain embodiments, the tumor comprises bladder cancer. In certain embodiments, the bladder cancer comprises Bladder Urothelial Carcinoma (BLCA).

In certain embodiments, the one or more genes comprise at least one or more protective effective genes as shown in Table 2.

In certain embodiments, the one or more genes comprise at least one or more risk effective genes as shown in Table 3.

In certain embodiments, the one or more genes comprise at least one or more genes as shown in Table 4. In certain embodiments, the one or more genes comprise at least one or more genes as shown in Table 5.

In certain embodiments, the device or method further comprises a step or module of determining the copy number variation of the one or more genes.

In certain embodiments, the method or device further comprises a step or module of determining the risk value of DNA methylation of one or more genes as shown in Table 8.

In certain embodiments, the method or device further comprises a step of module of determining the age of the subject.

In certain embodiments, determining the expression levels of one or more genes as shown in Table 1 in the subject or a biological sample derived from the subject in the device or method comprises: determining the average expression level of the genes as shown in Table 2 in the one or more genes; and determining the average expression level of the genes as shown in Table 3 in the one or more genes.

In certain embodiments, the device or method determines the tumor progression in the subject in accordance with Formula (I):

$$ln ( \frac{P ( Stages 1 )}{1 - P ( Stages 1 )} ) = Intercept + 0.0366 * a + 0.3386 * b + 0.3349 * c + 1.2193 * d + 0.0084 * e - 0.048 * f ; \qquad ( I )$$

wherein when j=Tumor Stage III, Intercept=0.9609; when j=Tumor Stage I/II, Intercept=−0.6617; a is the average expression level of the genes as shown in Table 2 in the one or more genes; b is the average expression level of the genes as shown in Table 3 in the one or more genes; c is the copy number variation of the one or more genes; d is the risk value of DNA methylation of the genes as shown in Table 8 in the one or more genes; e is the subject's age; and f is the subject's gender, wherein male is 0, and female is 1.

In another aspect, the present application provides a computer readable storage media having a computer program stored therein, wherein the computer program allows the computer to execute the determination method of the present application.

In another aspect, the present application provides a method of treating a tumor in a subject comprising: determining the tumor progression in the subject in accordance with the determination method of the present application; and administering an effective amount of treatment to the subject in accordance with the progression.

In another aspect, the present application provides a device of treating a tumor in a subject comprising: a) an analysis module capable of determining the expression levels of the one or more genes as shown in Table 1 in the subject or a biological sample derived from the subject; b) a determination module capable of determining the tumor progression in the subject in accordance with the expression level as measured in a); and c) a treatment module capable of administering an effective amount of treatment to the subject in accordance with the progression as determined in b).

Other aspects and advantages of the present disclosure will be readily apparent to those skilled in the art by reference to the following detailed description. The following detailed description merely shows and describes the exemplary embodiments of the present disclosure. Those skilled in the art will appreciate that the present disclosure enables the skilled persons in the art to make modifications to the particular embodiments as disclosed without departing the spirit and scope involved by the present application. Correspondingly, the drawings and the descriptions in the present application are only illustrative, other than restrictive.

### BRIEF DESCRIPTION OF DRAWINGS

The specific features of the inventions as claimed in the present application arc defined by the appended claims. The features and advantages of the present application can be better understood by reference to the exemplary embodiments and the accompany drawings as described in details below. The accompanying drawings are briefly described as follows.

FIG. 1 shows a schematic flowchart of the identification method and device of the present application.

FIGS. 2A-2D show a schematic graph of Kaplan-Meier curves of APOL2, BCL2L14, CSAD, and ORMDL1 expressions in two groups of different BLCA patients.

FIGS. 3A-3B show a gene ontology (GO) enrichment analysis of the protective effective genes and the risk effective genes among the genes which are essential to the survival of the BLCA patients.

FIGS. 4A-4C show a dynamic change of the correlation between the key genes in the BLCA patients in various tumor stages.

FIGS. 5A-5D show a functional module of gene co-expression network obtained by detection of WGCNA algorithm.

FIGS. 6A-6E show an analysis of copy number variation (CNV) in various stages of bladder cancer.

FIGS. 7A-7B show an exemplary result of DNA methylation analysis.

FIGS. 8A-8D show cellular signaling pathways enriching substantially the mutated genes in the BLCA sample.

FIGS. 9A-9E show an analysis of somatic mutations in various stages of bladder cancer.

FIGS. 10A-10C show an evolution of the microRNA-regulatory Network in various stages of bladder cancer.

FIG. 11 shows a forest plot of the ordered logistic regression in the integrated analysis.

### DETAILED DESCRIPTION OF THE INVENTION

Embodiments of the present application are illustrated hereinafter by way of specific embodiments, and those skilled in the art can readily understand other advantages and effects of the present application based on the present description.

In one aspect, the present application provides a device of identifying a biological indicator capable of evaluating a tumor progression comprising: 1) a clinical feature module capable of providing clinical feature of a patient with the tumor, wherein the clinical feature comprises the tumor stage of patient and/or the survival time of the patient; 2) a biological indicator module capable of providing at least one biological indicator derived from the patient; 3) a correlation determination module capable of determining a correlation between the at least one biological indicator of the individual patient with the clinical feature of the corresponding patient; and 4) an identification module capable of identifying the biological indicator which is determined to be correlated with the clinical feature in the module 3) as being capable of evaluating the tumor progression.

In another aspect, the present application provides a device of identifying a biological indicator capable of evaluating a tumor progression comprising a computer for identifying the biological indicator, said computer being programmed to executing the steps of: 1) providing clinical feature of a patient with the tumor, wherein the clinical feature comprises the tumor stage of patient and/or the survival time of the patient; 2) providing at least one biological indicator derived from the patient; 3) determining a correlation between the at least one biological indicator of the individual patient and the clinical feature of the same patient; and 4) identifying the biological indicator which is determined to be correlated with the clinical feature in 3) as being capable of evaluating the tumor progression.

In another aspect, the present application provides a method of identifying a biological indicator capable of evaluating a progression of a tumor comprising: 1) providing a clinical feature of a patient with the tumor, wherein the clinical feature comprises the tumor stage of patient and/or the survival time of the patient; 2) providing at least one biological indicator derived from the patient; 3) determining a correlation between the at least one biological indicator of the individual patient and the clinical feature of the corresponding patient; and 4) identifying the biological indicator which is determined to be correlated with the clinical feature in 3) as being capable of evaluating the tumor progression.

In the present application, the term “patient” generally refers to an individual having a characterization of disease, which may refer to either a symptom of disease, or in a case of prophylaxis to an undesirable physiological condition that cannot be changed. The individual may comprise male and/or female, and generally comprises humans or non-human animals, including, but not limited to, human, dog, cat, horse, sheep, goat, pig, cow, rabbit, rat, mouse, monkey, and the like. In certain embodiments, the patient is a human patient.

In the present application, the term “tumor” generally refers to an uncontrolled proliferation of some cells in the bodies due to abnormal pathological changes of cells, many of which tend to aggregate to form lumps. The tumors may be divided into benign tumors and malignant tumors. Among the malignant tumors, the proliferated cells aggregate to form lumps, and then spread to other sites. The tumors may be selected from the group consisting of: nasopharyngeal carcinoma, lip carcinoma, colorectal cancer, gallbladder cancer, lung cancer, liver cancer, cervical cancer, bone cancer, laryngeal carcinoma, melanoma, thyroid cancer, oropharyngeal cancer, brain tumor, bladder cancer, skin cancer, prostate cancer, breast cancer, esophagus cancer, glioma, tongue cancer, renal cancer, adrenocortical carcinoma, stomach cancer, angioma, pancreatic cancer, vagina cancer, uterine cancer, and lipoma. For example, the tumor can be bladder cancer, such as, Bladder Urothelial Carcinoma (BLCA).

Clinical Feature

In the present application, the term “clinical feature module” generally refers to a functional module capable of providing a clinical feature of a patient with the tumor. For example, the clinical feature module may comprise an information input and/or extraction unit capable of receiving and/or providing the clinical feature of the patient, including the tumor stage and/or the survival time of the patient.

In the present application, the term “clinical feature” generally refers to one or more indicator and/or parameters representing the clinical disease characteristics of the patient, e.g., the tumor stage and/or the survival time of the patient, and the like.

As used herein, the clinical feature module may comprise a reagent, an apparatus and/or an equipment capable of obtaining the tumor stage and/or the survival time of the patient. For example, the clinical feature module may comprise a reagent, apparatus, and/or equipment of detecting size, infiltration degree, and metastasis condition of the tumor (e.g., NMR-imaging, CT, estero- and gastro-scopy). As another example, the clinical feature module may comprise an apparatus and/or equipment of monitoring the survival time of the patient (e.g., a reagent, apparatus, and/or equipment for detection of a tumor marker). The tumor marker may be selected from the group consisting of: serum carcinoembryonic antigen (CEA), alpha fetoprotein (AFP), prostate specific antigen (PSA) and human chorionic gonadotropin (HCG).

In the present application, the term “tumor staging/stage” generally refers to a histopathological classification method of evaluating the tumor progression in accordance with the number and site of tumors in the patient. The tumor staging/stage may be used to describe the severity degree and the involvement scope of a malignant tumor depending on the degree of the primary tumor and the dissemination degree in an individual (e.g., in accordance with the TNM staging method suggested by the WHO). The tumor staging/stage may help a doctor to establish a corresponding therapy plan and understand the prognosis of the disease, while avoiding a circumstance of excessive or insufficient treatment. In general, the tumor is staged in accordance with the TNM staging method suggested by the World Health Organization (WHO). The English or numerical codes as used in the TNM staging method have the following meanings, respectively. T represents the extent and size of the primary tumor, the extent of infiltration, the presence or absence of metastasis, or the depth of infiltration, and is divided as 5 levels (from T0 to T4), wherein the greater number means the greater degree of the cancer progression. The staging methods vary depending on the cancer onset organs. N represents the circumstance of lymph node dissemination, and is divided as 4 levels (from N0 to N3), wherein the greater number means the greater degree of the cancer progression. M represents the presence or absence of metastasis, wherein M0 represents the absence of metastasis, and M1 represents the presence of distant metastasis. Clinically, the results of T, N, and M as described above are combined to determine the tumor stages. For example, the tumor stage may comprise Tumor Stage T, Tumor Stage II, Tumor Stage III, and Tumor Stage IV.

In the present application, the term “Tumor Stage I” generally refers to an early stage of tumor. In the present application, the term “Tumor Stage II” generally refers to a mild stage of tumor. In the present application, the term “Tumor Stage III” generally refers to a middle stage of tumor. In the present application, the term “Tumor Stage IV” generally refers to a complete stage of tumor.

In the present application, the term “survival time” refers to a total survival time of a post-treatment patient with tumor. The survival time may be associated with the tumor stage.

In the present application, the term “bladder cancer” generally refers to various malignant tumors of urinary bladder. The bladder cancer may comprise Bladder Urothelial Carcinoma (BLCA). The BLCA may be divided into non-muscle invasive bladder cancer and muscle-invasive bladder cancer. The bladder cancer has complicated causes, including both intrinsic genetic factors and extrinsic environmental factors. The two relatively common risk factors are smoking and occupational exposure to aromatic amine-based chemicals. In terms of clinical manifestations, about 90% or above of bladder cancer patients initially have a clinical manifestation of hematuria, usually manifested as painless, intermittent, gross hematuria, and sometimes microscopic hematuria. Hematuria may only occur once or last from one day to several days, and may alleviate or stop on its own. About 10% of bladder cancer patients may initially has an irritation sign of bladder, manifested as urinary frequency, urinary urgency, urinary pain, and difficulty of urination. The irritation sign of bladder is generally due to the reduction of bladder volume or the complicated infection caused by the tumor necrosis, the ulcer, the presence of large tumors or large number of tumors in the bladder, or the diffuse infiltration of bladder tumor into bladder wall.

As used herein, the bladder cancer may be staged into the following stages: Stage 0 bladder cancer (non-invasive papillary carcinoma and preinvasive carcinoma), Stage I, II, and III bladder cancers, and Stage IV bladder cancer. The therapies corresponding to the bladder cancers in different tumor stages comprise the following methods (see, the specification of the NIH (the National Cancer Institute)).

As for Stage 0 bladder cancer, the primary therapy comprises:
Trans-urethral resection via electrocautery,
   administration of intravesical chemotherapy immediately after surgery,
   administration of intravesical chemotherapy immediately after surgery, followed by administration of intravesical BCG or intravesical chemotherapy at regular intervals;
partial cystectomy;
radical cystectomy;
clinical practical of novel therapy.

As for Stage I bladder cancer, the primary therapy comprises:
Trans-urethral resection via electrocautery,
   administration of intravesical chemotherapy immediately after surgery;
   administration of intravesical chemotherapy immediately after surgery, followed by administration of intravesical BCG or intravesical chemotherapy at regular intervals;
partial cystectomy;
radical cystectomy;
clinical practical of novel therapy.

As for Stage II and Stage III bladder cancers, the primary therapy comprises:
radical cystectomy;
combined chemotherapy followed by radical cystectomy, and urinary diversion if required;
external radiotherapy, or external radiotherapy with chemotherapy;
partial cystectomy, or partial cystectomy with chemotherapy;
trans-urethral resection via electrocautery;
clinical trial of novel therapy.

As for Stage IV bladder cancer, the primary therapy comprises:
chemotherapy;
radical cystectomy alone, or followed by chemotherapy;
external radiotherapy, or external radiotherapy with chemotherapy;
urinary diversion or cystectomy as palliative therapy.

As for Stage IV bladder cancer that has spread to other sites of the body (such as, lung, bone, or liver), the therapy may comprise:
chemotherapy, or chemotherapy with local therapy (therapy or radiotherapy);
immunotherapy;
external radiotherapy as palliative therapy;
urinary diversion or cystectomy as palliative therapy;
clinical trial of novel anti-cancer drug.

Biological Indicator

In the present application, the term “biological indicator module” generally refers to a functional unit capable of providing at least one biological indicator derived from the patient. For example, the biological indicator module may provide an indicator and/or a feature reflecting the tumor stage of patient and/or the survival time of the patient at the molecular level.

For example, the biological indicator module may comprise a sample unit for obtaining a patient sample (e.g., a peripheral blood). For example, the biological indicator module may comprise a sample device for obtaining a patient sample (e.g., a device for obtaining a sample, such as, a blood taking needle or the like; and/or, a device for bearing a sample, such as, test tube or the like). For example, the biological indicator module may comprise a sample treatment device for obtaining the DNA of the patient by the treatment of the patient sample (e.g., a kit for extracting the whole blood DNA, a test tube, and a correlative device). As another example, the biological indicator module may further comprise an isolation unit capable of isolating a patient sample. For example, the biological indicator module may comprise a reagent for isolating cells (e.g., proteinase K) and a device for isolating cells (e.g., a centrifuge).

For example, the biological indicator module may comprise a sample treatment unit. For example, the sample treatment unit may comprise a reagent and a device for detecting the expression level of gene in the patient, a reagent and a device for detecting the copy number variation of gene in the patient, a reagent and a device for detecting the DNA methylation of gene in the patient, a reagent and a device for detecting the somatic mutation of gene in the patient, and a reagent and a device for detecting the microRNAs in the patient. As another example, the sample treatment unit may comprise a q-RT PCR kit, a MLPA (multiplex ligation-dependent probe amplification) kit, a kit for methylation profile analysis, a TruSeq Rapid Exomc Library kit and a kit for microarray analysis.

In the present application, the term “biological indicator” generally comprise one or more classes of indicators selected from the group consisting of: Class 1: the expression level of gene in the patient; Class 2: the copy number variation of gene in the patient; Class 3: the DNA methylation of gene in the patient; Class 4: the somatic mutation of gene in the patient; and Class 5: the microRNAs in the patient (microRNAs).

For example, the expression level of gene in the patient may be up-regulated, e.g., by about 10% or above, 20% or above, 30% or above, 40% or above, 50% or above, 60% or above, 70% or above, 80% or above, 90% or above, 100% or above, 120% or above, 140% or above, 160% or above, 180% or above; or 200% or above, as compared with the expression level in normal cells. For example, the expression level of gene in the patient may be down-regulated, e.g., to about 10% or less, 20% or less, 30% or less, 40% or less, 50% or less, 60% or less, 70% or less, 80% or less, 90% or less, 92% or less, 94% or less, 96% or less, 98% or less, or 99% or less, of the expression level in normal cells. For example, the copy number variation of gene in the patient may be increased, e.g., by about 0.1 times or above, about 0.5 times or above, about 1 time or above, about 2 times or above, about 3 times or above, about 4 times or above, about 5 times or above, about 6 times or above, about 7 times or above, about 8 times or above, about 9 times or above, or about 10 times or above, as compared with the expression level in normal cells. As another example, the copy number variation of gene in the patient may be decreased, e.g., by about 0.1 times or above, about 0.5 times or above, about 1 times or above, about 2 times or above, about 3 times or above, about 4 times or above, about 5 times or above, about 6 times or above, about 7 times or above, about 8 times or above, about 9 times or above, or about 10 times or above, as compared with the expression level in normal cells. For example, the DNA methylation level of gene in the patient may be increased, e.g., by about 0.1 times or above, about 0.5 times or above, about 1 times or above, about 2 times or above, about 3 times or above, about 4 times or above, about 5 times or above, about 6 times or above, about 7 times or above, about 8 times or above, about 9 times or above, or about 10 times or above, as compared with the DNA methylation level in normal cells. As another example, the DNA methylation level of gene in the patient may be decreased, e.g., by about 0.1 times or above, about 0.5 times or above, about 1 times or above, about 2 times or above, about 3 times or above, about 4 times or above, about 5 times or above, about 6 times or above, about 7 times or above, about times or above, about 9 times or above, or about 10 times or above, as compared with the DNA methylation level in normal cells.

In the present application, the term “expression level of gene” generally refers to the level of translating the information encoded by the gene to a gene product (e.g., RNA, protein). The expressed genes comprise genes to be transcribed to RNAs (e.g., mRNAs) which are subsequently translated to proteins and genes to be transcribed to non-coding functional RNAs (e.g., tRNAs, rRNA ribozymes, and the like) that are not translated to proteins. As used herein, “the expression level of gene” or “expression level” refers to the level (e.g., amount) of one or more products (e.g., RNAs, proteins) coded by a given gene in a sample or a reference standard.

In the present application, the term “copy number variation of gene” refers generally to the CNV (Copy Number Variation), which represents a phenomena that the slice repeats of genome and the number repeats in the genome differ among individuals in the population (see, Mccarroll, S. A et al., (2007). “Copy-number variation and correlation studies of human diseases”. Nature Genetics. 39: 37-42.). CNV is a repeat or deletion event, affecting a significant number of base pairs, and primarily occurs in human genomes. The copy number variations may generally be divided to two major categories: short repeats and long repeats. Short repeat sequences comprise primarily dinucleotide repeats (two repeating nucleotides, e.g., A-C-A-C-A-C . . . ) and trinucleotide repeats. Long repeat sequences comprise the repeats of the whole genes. The research data of CNV can not only provide additional evidences for evolution and natural selection, but also is used to develop therapies of various genetic diseases.

In the present application, the term “DNA methylation of gene” generally refers to a process of incorporating methyl into a DNA molecule (primarily, cytosine and adenine). Methylation may change the activity of a DNA fragment without changing the sequence. When the DNA methylation is located in a promoter of gene, it often serves to inhibit the transcription of gene. DNA methylation is essential to normal development, and associated with many key processes, including genomic imprinting, X-chromosome inactivation, and suppression of transposable factor, aging and carcinogenesis. Methylation of cytosine to form 5-methyl cytosine occurs at the same 5 position of the pyrimidine ring where the DNA base thymine methyl group is located; and the same position distinguishes between thymine and a similar RNA base uracil that does not contain a methyl group. The spontaneous deamination of 5-methyl cytosine converts it to thymine. It will lead to a T-G mismatching. The mechanism is repaired, and then it is changed back to the initial C-G pair; alternatively, it is possible to replace A with G, and change the initial C-G pair to T-A pair, thereby effectively changing the base and introducing a mutation. In the present application, the DNA methylation of gene may produce a DNA methylation mark, that is a genomic region of a specific methylation pattern with a specific biological state (e.g., tissue, cell type, individual), and considered as a potential functional region involved in gene transcriptional regulation.

In the present application, the term “somatic mutations of gene” generally refers to mutations occurring in cells other than the germ cell line, and are also known as acquired mutations. Somatic mutations do not cause genetic changes in the offsprings, but may cause changes in the genetic structure of some contemporary cells. Most somatic mutations have no phenotypic effect. The sporadic forms of malignant tumors may be caused by somatic mutations. Studies have shown that carcinogenesis of somatic cells is not necessarily accompanied with genetic structure change. When non-genetic substances, such as, proteins, RNAs, and biofilms, are changed, while these changes may also cause abnormal turn-off or turn-on of growth or differentiation-correlated genes, the cells may also be transformed into cancer cells at this time. Such viewpoint is called as extra-genetic regulation theory.

In the present application, the term “microRNAs” generally refers to non-coding RNAs having a length of about 22nt (microRNAs, briefly as miRNAs), which arc widely found from various organisms from viruses to humans. Such miRNAs have the ability of binding to mRNA to block the expression of protein-coding genes and preventing their translations into proteins. Mammalian miRNAs may have many unique targets. For example, the analysis of highly conversed miRNAs in vertebrates indicates that there are about 400 conversed targets on average for each miRNA. Similarly, an individual miRNA class may inhibit the production of hundreds of proteins. Studies have shown that chronic lymphocytic leukemia and B cell malignant tumors may be associated with miRNAs.

Correlation

In the present application, the term “correlation determination module” generally refers to a functional unit capable of determining a correlation between the at least one biological indicator of the individual patient with the clinical feature of the corresponding patient.

In the present application, the term “correlation” generally means that the at least one biological indicator of a patient in accordance with the present application exhibits a statistically significant correlation with the clinical feature of the corresponding patient. For example, one gene can be expressed at a higher or a lower level, and is associated with the state or result of tumor (e.g., bladder cancer).

For example, the correlation determination module may comprise a sample determination unit capable of determining a correlation between the at least one biological indicator of the individual patient with the clinical feature of the corresponding patient. For example, the correlation determination module may comprise a unit of determining the correlation between the expression level of gene and the clinical feature by performing a single variable regression analysis in relation to the clinical feature by use of the expression level of gene as the single variable (e.g., it may comprise a hardware, program and/or software capable of executing relevant instructions). For example, the correlation determination module may comprise a unit of determining the correlation between the expression level of gene and the clinical feature by performing a multiple variable regression analysis in relation to the clinical feature by use of the age of patient, the gender of patient, and/or the tumor stages of patient (e.g., it may comprise a hardware, program and/or software capable of executing the relevant instructions). As another example, the correlation determination module may further comprise a unit of determining the correlation between the expression level of gene and the clinical feature in accordance with the correlation coefficient values of individual genes obtained in the regression analysis (e.g., it may comprise a hardware, program and/or software capable of executing the relevant instructions).

As another example, the correlation determination module may further comprise a unit of determining the correlation between the expression level of gene and the clinical feature of each group, respectively, by determining the co-expression circumstance of genes specific for each tumor stage in accordance with the expression levels of the genes in various tumor stages of the patient, thereby classifying the genes into two or more groups in accordance with the co-expression circumstances of the genes (e.g., it may comprise a hardware, program and/or software capable of executing the relevant instructions). For example, the unit may utilize the WGCNA (Weighted Gene Co-Expression Network Analysis) algorithm to achieve at least a part of the functions thereof.

As another example, the correlation determination module may further comprise a unit of determining the correlation between the copy number variation of gene and the clinical feature in accordance with the variation frequency of genes in various tumor stages of the patient (e.g., it may comprise a hardware, program and/or software capable of executing the relevant instructions).

As another example, the correlation determination module may further comprise a unit of determining the correlation between the DNA methylation and the clinical feature in accordance with the DNA methylation, which is measured by performing a regression analysis in relation to the clinical feature by use of the degree of DNA methylation as the variable (e.g., it may comprise a hardware, program and/or software capable of executing the relevant instructions). As another example, the correlation determination module may further comprise a unit of determining the correlation between the DNA methylation and the clinical feature in accordance with the risk values of various DNA methylation sites, which are determined and identified as being correlated with the clinical feature by the correlation coefficients of the methylation sites obtained in the regression analysis as well as the methylation degree of the same methylation sites (e.g., it may comprise a hardware, program and/or software capable of executing the relevant instructions).

As another example, the correlation determination module may further comprise a unit of determining the correlation between the gene expression level of the somatic mutation and the clinical feature in accordance with the signaling pathway to which the genes containing the somatic mutation of the patient belong (e.g., it may comprise a hardware, program and/or software capable of executing the relevant instructions).

As another example, the correlation determination module may further comprise a unit of determining the correlation between the expression levels of the genes regulated by the microRNAs and the clinical feature and the clinical feature in accordance with the expression levels of the genes regulated by the microRNAs (e.g., it may comprise a hardware, program and/or software capable of executing the relevant instructions).

As another example, the correlation determination module may further comprise a unit of determining the correlation between the biological indicator and the clinical feature by determining the weight of two or more classes of the biological indicators to the clinical feature (e.g., it may comprise a hardware, program and/or software capable of executing the relevant instructions). For example, the unit may determine the weight by means of ordered logistic regression analysis.

As used herein, the at least one biological indicator may comprise the expression level of gene in the patient, and the determining the correlation between the expression level of gene and the clinical feature may comprise: performing a single variable regression analysis in relation to the clinical feature by use of the expression level of gene as the single variable, and identifying the genes of which the p value is less than or equal to a first threshold and the FDR value is less than or equal to a second threshold in the regression analysis as being correlated with the clinical feature.

In certain embodiments, the at least one biological indicator may comprise the expression level of gene in the patient, and the determining a correlation between the expression level of gene and the clinical feature comprises: a) performing a single variable regression analysis in relation to the clinical feature by use of the expression level of gene as the single variable, and identifying the genes of which the p value is less than or equal to a first threshold and the FDR value is less than or equal to a second threshold in the regression analysis as a first gene set correlated with the clinical feature.

In the present application, the term “first threshold ” generally refers to a cut-off value of the statistical significance of the determination results (i.e., a cut-off value of the p value) in the single variable regression analysis in relation to the clinical feature by use of the expression level of gene as the single variable. For example, the first threshold may be 0.09 or less. For example, the first threshold may be 0.08 or less, 0.07 or less, 0.06 or less, 0.05 or less, 0.045 or less, 0.04 or less, 0.03 or less, 0.02 or less, 0.01 or less, or 0.005 or less.

In the present application, the term “second threshold” generally refers to a threshold which the false discovery rate (FDR) is less than or equal to in the single variable regression analysis performed in relation to the clinical feature by use of the expression level of gene as the single variable. As used herein, the second threshold may be 0.5 or less. For example, the second threshold may be 0.4 or less, 0.3 or less, 0.2 or less, 0.1 or less, or 0.05 or less.

As used herein, if the expression level of gene satisfies both the first threshold and the second threshold, then the gene may be identified as a first gene set which is correlated with the clinical feature. As used herein, if the expression level of gene satisfies both the first threshold and the second threshold, then the expression level of gene may be correlated with the clinical feature, and/or the gene may be used as one of the biological indicators for evaluating the tumor progression.

As used herein, the at least one biological indicator may comprise the expression level of gene in the patient, and the determining a correlation between the expression level of gene and the clinical feature comprise performing a multiple-variable regression analysis against the clinical feature, and identifying the genes of which the FDR value is less than or equal to a third threshold in the regression analysis as being correlated with the clinical feature, and wherein and the multiple variables comprise the expression level of gene in the patient, the age of the patient, the gender of the patient, and/or the tumor stages of the patient.

In certain embodiments, the determining the correlation between the expression level of gene and the clinical feature further comprises: b) performing a multiple-variable regression analysis in relation to the clinical feature, and identifying the genes of which the FDR value is less than or equal to a third threshold in the regression analysis as a second gene set correlated with the clinical feature, and wherein the multiple variables comprise the expression level of the individual genes in the first gene set, the age of the patient, the gender of the patient, and the tumor stage of the patient.

In the present application, the term “third threshold” generally refers to a threshold which the false discovery rate (FDR) is less than or equal in a multiple-variable regression analysis performed in relation to the clinical feature. Among others, the multiple variables may be selected from the group consisting of: the expression level of gene in the patient, the age of patient, the gender of patient, and/or the tumor stages of the patient. As used herein, the third threshold may be 0.2 or less. For example, the third threshold may be 0.2 or less, 0.15 or less, 0.1 or less, or 0.05 or less.

As used herein, if the expression level of gene satisfies the third threshold, then the gene may be identified as a second gene set which is correlated with the clinical feature. For example, the genes of the second gene set may be selected from those listed in Table 1. For example, the number of gene in the second gene set may be 1078.

As used herein, the at least one biological indicator may comprise the expression level of gene in the patient, and the determining the correlation between the expression level of gene and the clinical feature further comprises: classifying the genes into protective effective genes and risk effective genes in accordance with the correlation coefficient values of the individual genes obtained in the multiple variable regression analysis, wherein the correlation coefficient value of the protective effective genes may be negative, and the correlation coefficient value of the risk effective genes may be positive.

As used herein, the determining the correlation between the expression level of gene and the clinical feature may further comprise: c) classifying the genes into protective effective genes and risk effective genes in accordance with the correlation coefficient values of the individual genes obtained in the multiple variable regression analysis, wherein the protective effective genes have negative correlation coefficient values, and the risk effective genes have positive correlation coefficient values.

In the present application, the term “protective effective gene” generally refers to genes of which the expression level is in positive correlation with the survival time of the patient, or in negative correlation with the progression degree of tumor (e.g., the progression of the tumor stage). For example, in the multiple-variable regression analysis of the present application, the correlation coefficient value between the expression level of the protective effective genes and the clinical feature (e.g., the tumor stage) may be negative. As used herein, the protective effective genes may be selected from those listed in Table 2. As used herein, the number of the protective effective gene may be 356. The expression level of the protective effective genes may be down-regulated during the progression of the tumor. For example, the protective effective genes may be in negative correlation with the tumor stages.

In the present application, the term “risk effective genes” generally refers to genes of which the expression level is in negative correlation with the survival time of the patient, or in positive correlation with the progression degree of tumor (e.g., the progression of the tumor stage). For example, in the multiple-variable regression analysis of the present application, the correlation coefficient value between the expression level of the risk effective genes and the clinical feature (e.g., the tumor stage) may be positive. As used herein, the risk effective genes may be selected from those listed in Table 3. As used herein, the number of the risk effective genes may be 722. The expression level of the risk effective genes may be up-regulated during the progression of the tumor. For example, the risk effective genes may be in positive correlation with the tumor stage.

As used herein, the at least one biological indicator may comprise the expression level of gene in the patient, and determining the correlation between the expression level of gene and the clinical feature further comprises that determining the expression level of gene in the patient in the individual tumor stage, determining accordingly the co-expression circumstances of genes which are specific for tumor staging, classifying the genes into two or more groups in accordance with the co-expression circumstances of the genes, and determining the correlation between the expression level of gene of each group and the clinical feature. For example, the genes may be classified into two or more groups by identifying the co-expression relation of individual genes in a certain tumor stage, and/or identifying the variation of such co-expression relationship between various tumor stages, wherein the genes of each group may present a specific co-expression pattern of the tumor stage. Next, analyzing the correlation between the genes of each group and the clinical feature (e.g., the survival time of the patient and/or the tumor stages) (e.g., via the single-variable and/or multiple-variable regression analysis as described in the present application), thereby identifying the genomes having the desired correlations.

As used herein, the determining the correlation between the expression level of gene and the clinical feature may further comprise: determining the expression level of the individual genes of the second gene set in various tumor stage, determining accordingly the co-expression circumstances of genes which are specific for tumor staging, classifying the genes of the second gene set into two or more groups in accordance with the co-expression circumstances of genes, and determining the correlation between the expression level of gene of each group and the clinical feature. For example, the genes of the second gene set may be classified into two or more groups by identifying the co-expression relationship of individual genes in a certain tumor stage, and/or identifying the variation of such co-expression relationship between various tumor stages, wherein the genes of each group may present a specific co-expression profile of the tumor stage. Next, the genes of each group may be analyzed for their correlations with the clinical feature (e.g., the survival time of the patient and/or the tumor stages) (e.g., via the single-variable and/or the multiple-variable regression analysis as described in the present application), thereby identifying the genomes having the desired correlations.

In the present application, the term “co-expression of gene” refers generally to a tendency that a variety of genes of the second gene set can exhibit a similar expression level in a certain stage of the tumor (e.g., the expression levels have the same or similar tendency in a certain tumor, such as, up-regulated in Tumor Stage I), thereby classifying the genes of the second gene set into two more groups (e.g., 2 groups or more, 3 groups or more, 4 groups or more, 5 groups or more, 6 groups or more, 7 groups or more, 8 groups or more, 9 groups or more, 10 groups or more, or more) in accordance with the co-expression of gene, so that the expression level of gene in each group is correlated with the clinical feature. For example, the co-expression of gene may be determined by use of WGCNA algorithm.

As used herein, the at least one biological indicator may comprise the copy number variation of gene in the patient, and determining the correlation between the copy number variation of gene and the clinical feature comprises: comparing the copy number variation frequencies of gene in the patient in various tumor stages.

In certain embodiments, the at least one biological indicator further comprises the copy number variation of gene in the patient, and determining the correlation between the copy number variation of gene and the clinical feature comprises: comparing the copy number variation frequencies of the genes of the second gene set in various tumor stages.

As used herein, the at least one biological indicator may comprise the DNA methylation of gene in the patient, and the determining the correlation between the DNA methylation and the clinical feature comprises: performing a regression analysis in relation to the clinical feature by use of the degree of DNA methylation as the variable, and identifying the DNA methylation of which the p value is less than or equal to a fourth threshold in the regression analysis as being correlated with the clinical feature.

In certain embodiments, the at least one biological indicator further comprises the DNA methylation of gene in the patient, and the determining the correlation between the DNA methylation and the clinical feature comprises: determining the DNA methylation sites of the genes of the second gene set and the DNA methylation degrees at the individual sites, performing a regression analysis in relation to the clinical feature by use of the DNA methylation degree as the variable, and identifying the DNA methylations of which the p value is less than or equal to a fourth threshold in the regression analysis as a first DNA methylation set correlated with the clinical feature.

In the present application, the term “fourth threshold” generally refers to a threshold which the p value is less than or equal to in the regression analysis performed in relation to the clinical feature by use of the DNA methylation degree of gene as the variable (e.g., a cut-off value of the p value exhibiting the statistical significance). As used herein, the fourth threshold may be less than 0.2. For example, the fourth threshold may be less than 0.15, less than 0.1, less than 0.05, less than 0.01, or less than 0.005.

In the present application, if the p value is less than or equal to the fourth threshold in the regression analysis of the DNA methylation degree of the genes of the second gene set, then the DNA methylation may be identified as a first DNA methylation set which is correlated with the clinical feature. As used herein, the first DNA methylation set may be selected from genes as listed in Table 8. For example, the first DNA methylation set may comprise the DNA methylation events in 23 genes.

In the present application, the determining the correlation between the DNA methylation and the clinical feature may further comprise: determining the risk value of various DNA methylation sites which are identified as being correlated with the clinical feature, wherein the risk values are determined based on the correlation coefficients of the methylation sites obtained in the regression analysis, as well as the methylation degrees of the methylation sites.

In certain embodiments, the determining the correlation between the DNA methylation and the clinical feature further comprises determining the risk values of various DNA methylation sites in the first DNA methylation set, wherein the risk values are determined based on the correlation coefficients of the methylation sites obtained in the regression analysis, as well as the methylation degrees of the methylation sites. For example, the risk value of a certain DNA methylation event may be a linear combination of the correlation coefficient of the methylation site obtained in the regression analysis with the value of the methylation degree of the methylation site.

As used herein, the at least one biological indicator may comprise the somatic mutation of gene in the patient, and the determining the correlation between the somatic mutation and the clinical feature comprises: determining the signal pathway of the gene containing the somatic mutation, and/or determining the correlation between the expression level of the gene containing the somatic mutation and the clinical feature.

In certain embodiments, the at least one biological indicator further comprises the somatic mutation of gene in the patient, and the determining the correlation between the somatic mutation and the clinical feature comprises: determining the somatic mutation contained in the gene in the second gene set, and determining the signal pathway of the gene containing the somatic mutation.

In the present application, the signaling pathway may comprise PI3K/AKT pathway, Ras pathway, Rap1 pathway and MAPK pathway. As used herein, the signaling pathway may be confirmed to be correlated with a tumor.

In the present application, the at least one biological indicator may comprise the microRNAs in the patient, and the determining the correlation between the microRNA and the clinical feature comprises: determining the correlation between the expression level of the gene regulated by the microRNA and the clinical feature, and determining the correlation between the expression level of the microRNA in the patient and the expression level of the gene regulated by the microRNA.

In certain embodiments, the at least one biological indicator may comprise the microRNAs in the patient, and the determining the correlation between the microRNA and the clinical feature comprises: determining the microRNA regulating the gene of the second gene set, and determining the correlation between the expression level of the microRNA in the patient and the expression level of gene regulated by the microRNA, identifying the microRNA having a correlation higher than a fifth threshold as a first microRNA set correlated with the clinical feature.

In the present application, the term “fifth threshold” generally refers to a cut-off value of determining the statistical significance of the correlation. As used herein, the fifth threshold may be less than −0.1. For example, the fifth threshold may be less than −0.15, less than −0.2, less than −0.25, less than −0.3, less than −0.35, less than —0.4, or less than −0.45. As used herein, if the correlation coefficient is less than the fifth threshold, then it may be considered that there is a significant correlation between the expression level of genes regulated by the microRNAs and the expression level of the microRNAs. For example, the microRNAs and the genes interacting may be paired as a regulation pair (a microRNA-gene regulation pair). Thus, the fifth threshold may reflect the matching degree of microRNA with a gene regulated thereby. As used herein, the fifth threshold may vary with the tumor stage.

In the present application, the term “first microRNAs set” may comprise microRNAs having the correlation higher than the fifth threshold. As used herein, the first microRNAs set may be selected from those as listed in Table 10.

As used herein, the at least one biological indicator may comprise two or more classes of the biological indicators, and the determining the correlation between the biological indicator and the clinical feature comprises determining the weights of various biological indicators to the clinical feature. For example, the weight may be determined by means of ordered logistic regression analysis.

As used herein, determining the correlation between the biological indicator and the clinical feature may comprise: determining the weight of the following biological indicators to the clinical feature by an ordered logistic regression analysis, respectively: the expression level of genes of the second gene set, the copy number variation of the genes of the second gene set, the risk value of the DNA methylation sites of the first DNA methylation set. For example, the respective weights of the expression level of the protective effective genes and the expression level of the risk effective genes of the second gene set may be determined, respectively.

In the present application, the term “weight” generally refers to the relative importance of a certain indicator (e.g., the biological indicator) in the overall evaluation (e.g., the evaluation of tumor progression).

In another aspect, the present application further provides a computer readable storage medium having a computer program stored, wherein the computer program allows the computer to execute the method as described in the present application.

In the present application, the term “computer readable storage medium” generally refers to a media for storing certain parameters or data contained in a computer storage. The computer storage medium may comprise, e.g., semi-conductors, magnetic cores, magnetic drums, magnetic tapes, laser discs, and the like.

In the present application, the term “identification module” generally refers to a functional unit capable of identifying the biological indicator which is identified as being correlated with the clinical feature in the correlation determination module as being capable of evaluating the tumor progression.

For example, the identification module may comprise a program, reagent, and/or device capable of identifying the biological indicator as being capable of evaluating the tumor progression.

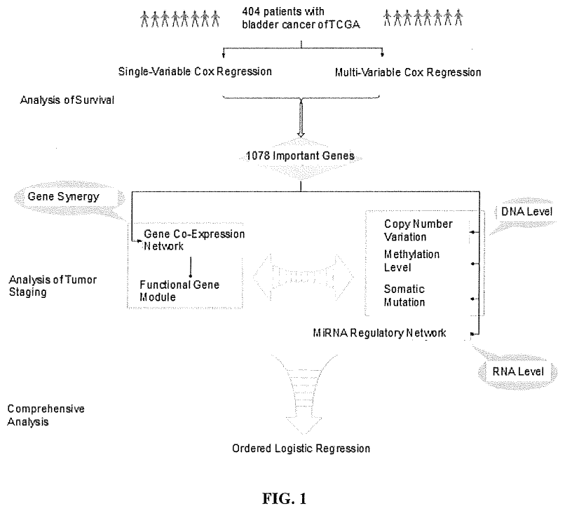

In the present application, the identifying a biological indicator capable of evaluating a tumor progression may be divided into three phases (as shown in FIG. 1): Phase I: Identifying 1078 key genes by a large-scale Cox regression model (i.e., single- and multi-variable Cox regression models) in accordance with the effects of genes on survival status in a patient with tumor (e.g., a patient with bladder cancer) obtained from TCGA, followed by analyzing these genes for their protectiveness or harmfulness in accordance with the relationships of the genes with the survival rate of the patient and/or the tumor stages in various stages of tumors (e.g., bladder cancer). Phase II: Analyzing the state-specific co-expression profile of genes in various stages of the tumor (e.g., bladder cancer), and accordingly dividing the 1078 key genes into a variety of sub-groups wherein the genes in each sub-group presents the same or similar stage-specific co-expression pattern, followed by determining the correlation between the genes in the individual sub-groups and the survival rate of the patient and/or the tumor stages, thereby identifying the gene sub-group which is the most correlative with the tumor progression in the 1078 key genes. Phase III: Analyzing the correlations between the progression (e.g., the survival rate of the patient and/or the tumor stages) of the tumor (e.g., bladder cancer) and other biological indicators of the patient, such as, the copy number variation of 1078 key genes, the DNA methylation circumstance, the somatic mutations, and the microRNA regulatory Network, and the like, respectively, thereby identifying one or more additional biological indicators capable of exhibiting the correlation. Phase IV: Performing an integrated analysis on the comprehensive correlation between the identified indicators and the progression (e.g., the survival rate of the patient and/or the tumor stages) of the tumor (e.g., bladder cancer). By the aforesaid studies, the present application provides a systemic and reasonable manner to comprehensively analyze the biological indicator data and the clinical feature data of the patient, thereby revealing the characteristic index of the progression of cancer (e.g., bladder cancer).

Device or Method of Determining Tumor Progression

In another aspect, the present application provides a device of determining a tumor progression in a subject comprising: a) an analysis module capable of determining the expression levels of the genes as shown in Table 1 in the subject or a biological sample derived from the subject; and b) a determination module capable of determining the tumor progression in the subject in accordance with the expression level as measured in a).

The present application further provides a device of determining a tumor progression in a subject comprising a computer for determining a tumor progression in a subject, said computer being programmed to executing the steps of: a) determining the expression levels of one or more genes as shown in Table 1 in the subject or a biological sample derived from the subject; and b) determining the tumor progression in the subject in accordance with the expression level as measured in a).

In another aspect, the present application provides a method of determining a tumor progression in a subject comprising: a) determining the expression levels of one or more genes as shown in Table 1 in the subject or a biological sample derived from the subject; and b) determining the tumor progression in the subject in accordance with the expression level as measured in a).

In the present application, the term “an analysis module” generally refers to a functional unit capable of determining the expression levels of the genes as shown in Table 1 in the subject or a biological sample derived from the subject.

For example, the analysis module may comprise a sample unit of obtaining a sample (e.g., a peripheral blood) from a subject. For example, the analysis module may comprise a sample device of obtaining a sample from a subject (e.g., a device of obtaining a sample, such as, blood taking needle and the like; and/or, a device of bearing a sample, such as, test tube and the like). For example, the analysis module may comprise a sample treatment device of obtaining the DNA of a subject by treating a sample from the patient (e.g., a kit for extracting the whole blood DNA, a test tube, and a correlative device). As another example, the analysis module may further comprise an isolation unit capable of isolating a sample from a subject. For example, the analysis module may comprise a reagent of isolating cells (e.g., proteinase K) and a device of isolating cells (e.g., centrifuge).

For example, the analysis module may comprise a reagent and equipment of detecting the expression levels of one or more genes as shown in Table 1 in the subject or a biological sample derived from the subject. For example, the analysis module may comprise a q-RT PCR kit and a q-RT PCR instrument.

In the present application, the term “a determination module” generally refers to a functional unit of determining the tumor progression in the subject in accordance with the expression level as determined in the analysis module.

For example, the determination module may comprise a sample determination unit capable of determining the tumor progression in the subject in accordance with the expression level as determined in the analysis module.

For example, the tumor progression may comprise the stages of the tumor and/or the survival rate of the subject.

For example, the tumor stage may be selected from the group consisting of: Tumor Stage I, Tumor Stage II, Tumor Stage III, and Tumor Stage IV.

For example, the tumor may comprise bladder cancer. As another example, the bladder cancer may comprise Bladder Urothelial Carcinoma (BLCA).

In the present application, the one or more genes may comprise at least one or more protective effective genes as shown in Table 2.

In the present application, the one or more genes may comprise at least one or more risk effective genes as shown in Table 3.

In the present application, the one or more genes may comprise at least one or more genes as shown in Table 4. For example, the expression levels of the genes as shown in Table 4 may have a negative correlation coefficient value with the tumor stage. For example, the expression levels of the genes as shown in Table 4 (e.g., 93% or above, 94% or above, 95% or above, 96% or above, 97% or above, 98% or above, 99% or above; or 100% of the genes in Table 4) may have negative correlation coefficient values with the stages of bladder cancer.

In the present application, the one or more genes may comprise at least one or more genes as shown in Table 5. For example, the expression levels of the genes in Table 5 can have a positive correlation coefficient value with the tumor stages. For example, the expression levels of the genes in Table 5 can have positive correlation coefficient value with the stages of bladder cancer.

In the present application, the device or method may further comprise a step of module of determining the copy number variation of the one or more genes. For example, the determining the copy number variation may comprise the step of performing an analysis by use of the copy number variation data in the Broad GDAC Firehose. Of those, the data are derived from samples in various stages of bladder cancer of a patient.

In the present application, the method or device may further comprise a step or module of determining the risk values of DNA methylation of the one or more genes in Table 8.

In the present application, the risk values are generally determined based on the correlation coefficients of the methylation site obtained in the regression analysis and the methylation degree of the methylation site. For example, the risk value may be determined in accordance with a method comprising the following steps: it may be defined as a linear combination of the methylation levels (i.e., 13 value) with the corresponding coefficients of the 23 DNA methylation genes in regularized Cox regression (e.g., the genes in the first DNA methylation set of the present application, or the genes as shown in Table 8); and then all patient were subject to risk scoring in accordance with the median risk value so as to divide the patients into a high-risk group and a low-risk group, which were subsequently subject to Kaplan-Meier analysis and log-rank Test.

In the present application, the method or device further comprises a step or module of determining or providing the age of the subject. For example, the step or module may comprise or execute the steps of: asking for the age of the patient, investigating the medical records of the patient or determining the bone ages, and the like.

In the present application, the determining the expression levels of one or more genes as shown in Table 1 in the subject or a biological sample derived from the subject in the device or method may comprise: determining the average expression level of the genes as shown in Table 2 in the one or more genes; and determining the average expression level of the genes as shown in Table 3 in the one or more genes. For example, the expression levels of on eor more genes as shown in Table 1 in the subject or a biological sample derived from the subject may be determined based on the average expression level of one or more (e.g., 1 or more, 2 or more, 4 or more, 6 or more, 8 or more, 10 or more, 20 or more, 50 or more, 100 or more, 200 or more or 500 or more) genes in Table 2 and Table 3 as measured, respectively.

Integrated Determination

In the present application, the device or method may determine the tumor progression in the subject in accordance with Formula (I):

$$ln ( \frac{P ( Stages 1 )}{1 - P ( Stages 1 )} ) = Intercept + 0.0366 * a + 0.3386 * b + 0.3349 * c + 1.2193 * d + 0.0084 * e - 0.048 * f \qquad ( I )$$

wherein when j=Tumor Stage III, Intercept=0.9609; when j=Tumor Stage I/II, Intercept=−0.6617; a is the average expression level of the one or more genes as shown in Table 2 in the one or more genes; b is the average expression level of the one or more genes as shown in Table 3 in the one or more genes; c is the copy number variation of the one or more genes; d is the risk value of DNA methylation of the one or more genes as shown in Table 8 in the one or more genes; e is the subject's age; and f is the subject's gender, wherein male is 0, and female is 1.

In another aspect, the present application provides a computer readable storage media having a computer program stored therein, wherein the computer program may allow the computer to execute the aforesaid determination.

Method of Treating Tumor

In another aspect, the present application provides a method of treating a tumor in a subject comprising: determining the tumor progression in the subject in accordance with the determination method of the present application; and administering an effective amount of treatment to the subject in accordance with the progression.

For example, the tumor may comprise bladder cancer (e.g., Bladder Urothelial Carcinoma (BLCA)). As another example, the tumor progression may be selected from the group consisting of: Tumor Stage I, Tumor Stage II, Tumor Stage III, and Tumor Stage IV.

For example, when the subject has Stage I bladder cancer, the treatment may comprise: Trans-urethral resection via electrocautery, intravesical chemotherapy, partial cystectomy, and radical cystectomy. For example, when the subject has Stages II and Stage III bladder cancer, the treatment may comprise: radical cystectomy, combined chemotherapy followed by radical cystectomy, radiotherapy, partial cystectomy and Trans-urethral resection via electrocautery. For example, when the subject has Stage IV bladder cancer, the treatment may comprise: chemotherapy, radical cystectomy alone or followed by chemotherapy, external radiotherapy, or external radiotherapy with chemotherapy and palliative treatment (e.g., urinary diversion or cystectomy).

In another aspect, the present application provides a device of treating a tumor in a subject comprising: a) an analysis module capable of determining the expression levels of the genes as shown in Table 1 in the subject or a biological sample derived from the subject; b) a determination module capable of determining the tumor progression in the subject in accordance with the expression level as measured in a); and c) a treatment module capable of administering an effective amount of treatment to the subject in accordance with the progression as determined in b).

In the present application, the term “treatment module” generally refers to a functional unit capable of determining and/or performing an administration of an effective amount of treatment to the subject in accordance with the tumor progression as determined in the determination module.

For example, the treatment module may comprise a reagent, agent, apparatus, and equipment: surgery for tumor resection, chemotherapy, radiotherapy, biologically targeted therapy, and palliative treatment. Of those, the palliative treatment may be a therapeutic method of controlling the symptoms affecting the life quality, such as, including pain, anorexia, constipation, fatigue, dyspnea, vomiting, cough, dry mouth, diarrhea, dysphagia, and the like, together with paying attention to psychic and mental problems. For example, the cancer may be bladder cancer, and the biologically targeted therapy may comprise administering, e.g., IL2 and/or IFN-α2a.

For example, the treatment module may comprise administering an effective amount of an agent to the subject. The “effective amount” may be an amount of drug that relieve or eliminate the diseases or symptoms of the subject. Typically, the particular effective amount may be determined in accordance with the weight, age, gender, diet, excretion rate, past medical history, current treatment of the patient, administration time, dosage form, administration manner, administration route, combination of drugs, health condition and potential of cross infection of the patient, allergy, hypersensitivity, and side-effects of the subject, and/or the degrees of tumor staging. Persons skilled in the art (e.g., physicians or veterinarians) may proportionally increase or decrease the effective amount in accordance with these or other conditions or requirements.

In the present application, the term “about” generally refers to a variation within 0.5%-10% of a specified value, e.g., a variation within 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of the specified value.

The present application further relates to the following embodiments: 1. A device of identifying a biological indicator capable of evaluating a tumor progression comprising:

1) a clinical feature module capable of providing clinical feature of a patient with the tumor, wherein the clinical feature comprise the tumor stage of patient and/or the survival time of the patient;

2) a biological indicator module capable of providing at least one biological indicator derived from the patient;

3) a correlation determination module capable of determining a correlation between the at least one biological indicator of the individual patient with the clinical feature of the corresponding patient; and

4) an identification module capable of identifying the biological indicator which is determined to be correlated with the clinical feature in the module 3) as being capable of evaluating the tumor progression.

2. A device of identifying a biological indicator capable of evaluating a tumor progression comprising a computer for identifying the biological indicator, said computer being programmed to executing the steps of:

1) providing clinical feature of a patient with the tumor, wherein the clinical feature comprise a tumor stage of the patient and/or a survival time of the patient;

2) providing at least one biological indicator derived from the patient;

3) determining a correlation between the at least one biological indicator of the individual patient and the clinical feature of the corresponding patient; and

4) identifying the biological indicator which is determined to be correlated with the clinical feature in 3) as being capable of evaluating the tumor progression.

3. A method of identifying a biological indicator capable of evaluating a progression of a tumor comprising:

1) providing a clinical feature of a patient with the tumor, wherein the clinical feature comprise a tumor stage of the patient and/or a survival time of the patient;

2) providing at least one biological indicator derived from the patient;

3) determining a correlation between the at least one biological indicator of the individual patient and the clinical feature of the corresponding patient; and

4) identifying the biological indicator which is determined to be correlated with the clinical feature in 3) as being capable of evaluating the tumor progression.

4. The method or device according to any one of embodiments 1-3, wherein the tumor comprises bladder cancer.

5. The method or device according to embodiment 4, wherein the bladder cancer comprises Bladder Urothelial Carcinoma (BLCA).

6. The method or device in accordance with any one of embodiments 1-5, wherein the tumor stage is selected from the group consisting of: Tumor Stage I, Tumor Stage II, Tumor Stage III, and Tumor Stage IV.

7. The method or device in accordance with any one of embodiments 1-6, wherein the at least one biological indicator comprises one or more classes of indicators selected from the group consisting of:

Class 1: the expression level of gene in the patient;

Class 2: the copy number variation of gene in the patient;

Class 3: the DNA methylation of gene in the patient;

Class 4: the somatic mutation of gene in the patient; and

Class 5: the microRNAs in the patient.

8. The method or device in accordance with embodiment 7, wherein the at least one biological indicator comprises the expression level of gene in the patient, and the determining a correlation between the expression level of gene and the clinical feature comprises: performing a single variable regression analysis in relation to the clinical feature by use of the expression level of gene as the single variable, and identifying the genes of which the p value is less than or equal to a first threshold and the FDR value is less than or equal to a second threshold in the regression analysis as being correlated with the clinical feature.

9. The method or device in accordance with any one of embodiments 7-8, wherein the at least one biological indicator comprises the expression level of gene in the patient, and the determining a correlation between the expression level of gene and the clinical feature comprise performing a multiple-variable regression analysis against the clinical feature, and identifying the genes of which the FDR value is less than or equal to a third threshold in the regression analysis as being correlated with the clinical feature, and wherein and the multiple variables comprise the expression level of gene in the patient, the age of the patient, the gender of the patient, and/or the tumor stages of the patient.

10. The method or device in accordance with any one of embodiments 8-9, wherein the at least one biological indicator comprises the expression level of gene in the patient, and the determining the correlation between the expression level of gene and the clinical feature further comprises dividing the genes into protective effective genes and risk effective genes in accordance with the correlation coefficient values of individual genes obtained in the regression analysis, wherein the protective effective genes have negative correlation coefficient values, and the risk effective genes has positive correlation coefficient values.

11. The method or device in accordance with any one of embodiments 7-10, wherein the at least one biological indicator comprises the expression level of gene in the patient, and the determining the correlation between the expression level of gene and the clinical feature further comprises that determining the expression level of gene in the patient in the individual tumor stage, determining accordingly the co-expression circumstances of genes which are specific for tumor staging, classifying the genes into two or more groups in accordance with the co-expression circumstances of the genes, and determining the correlation between the expression level of gene of each group and the clinical feature respectively.

12. The method or device in accordance with embodiment 11 comprising classifying the genes into two or more groups in accordance with the co-expression circumstances of the genes by use of WGCNA algorithm.

13. The method or device in accordance with any one of embodiments 7-12, wherein the at least one biological indicator comprises the copy number variation of gene in the patient, and the determining the correlation between the copy number variation of gene and the clinical feature comprises: comparing the copy number variation frequencies of gene in the patient in various tumor stages.

14. The method or device in accordance with any one of embodiments 7-13, wherein the at least one biological indicator comprises the DNA methylation of gene in the patient, and the determining the correlation between the DNA methylation and the clinical feature comprises: performing a regression analysis in relation to the clinical feature by use of the degree of DNA methylation as the variable, and identifying the DNA methylation of which the p value is less than or equal to a fourth threshold in the regression analysis as being correlated with the clinical feature.

15. The method or device in accordance with embodiment 14, wherein the determining the correlation between the DNA methylation and the clinical feature further comprises: determining the risk values of various DNA methylation sites which are determined to be correlated with the clinical feature, wherein the risk values are determined based on the correlation coefficients of the methylation sites obtained in the regression analysis, as well as the methylation degrees of the methylation sites.

16. The method or device in accordance with any one of embodiments 7-15, wherein the at least one biological indicator comprises the somatic mutation of gene in the patient, and the determining the correlation between the somatic mutation and the clinical feature comprises: determining the signal pathway of the gene containing the somatic mutation, and/or determining the correlation between the expression level of the gene containing the somatic mutation and the clinical feature.

17. The method or device in accordance with any one of embodiments 7-16, wherein the at least one biological indicator comprises the microRNAs in the patient, and the determining the correlation between the microRNA and the clinical feature comprises: determining the correlation between the expression level of the gene regulated by the microRNA and the clinical feature, and determining the expression level of the microRNA in the patient and the expression level of the gene regulated by the microRNA.

18. The method or device in accordance with any one of embodiments 7-17, wherein the at least one biological indicator comprises two or more classes of the biological indicators, and the determining the correlation between the biological indicator and the clinical features comprises determining the weights of various biological indicators to the clinical feature.

19. The method or device in accordance with embodiment 18 comprising determining the weight by means of ordered logistic regression analysis.

20. The method or device in accordance with any one of embodiments 1-19, wherein the at least one biological indicator comprises the expression level of gene in the patient, and the determining a correlation between the expression level of gene and the clinical feature comprises:

a) performing a single variable regression analysis in relation to the clinical feature by use of the expression level of gene as the single variable, and identifying the genes of which the p value is less than or equal to a first threshold and the FDR value is less than or equal to a second threshold in the regression analysis as a first gene set associated with the clinical feature.

21. The method or device in accordance with embodiment 20, wherein the determining the correlation between the expression level of gene and the clinical feature further comprises:

b) performing a multiple-variable regression analysis in relation to the clinical feature, and identifying the genes of which the FDR value is less than or equal to a third threshold in the regression analysis as a second gene set correlated with the clinical feature, and wherein the multiple variables comprise the expression level of the individual genes in the first gene set, the age of the patient, the gender of the patient, and the tumor stage of the patient.

22. The method or device in accordance with embodiment 21, wherein the determining the correlation between the expression level of gene and the clinical feature further comprises:

c) classifying the genes into protective effective genes and risk effective genes in accordance with the correlation coefficient values of the individual genes obtained in the multiple variable regression analysis, wherein the protective effective genes have negative correlation coefficient values, and the risk effective genes has positive correlation coefficient values.

23. The method or device in accordance with any one of embodiments 21-22, wherein the determining the correlation between the expression level of gene and the clinical feature further comprises: determining the expression levels of the individual genes of the second gene set in various tumor stages, determining accordingly the co-expression circumstances of genes which are specific for tumor staging, classifying the genes of the second gene set into two or more groups in accordance with the co-expression circumstances of genes, and determining the correlation between the expression level of gene of each group and the clinical feature.

24. The method or device in accordance with embodiment 23, wherein the genes of the second gene set are divided into two or more groups in accordance with the co-expression circumstance of genes by use of WGCNA algorithm.

25. The method or device in accordance with any one of embodiments 21-24, wherein the at least one biological indicator further comprises the copy number variation of gene in the patient, and the determining the correlation between the copy number variation of gene and the clinical feature comprises: comparing the copy number variation frequencies of the genes of the second gene set in various tumor stages.

26. The method or device in accordance with any one of embodiments 21-25, wherein the at least one biological indicator further comprises the DNA methylation of gene in the patient, and the determining the correlation between the DNA methylation and the clinical feature comprises: determining the DNA methylation sites of the genes of the second gene set and the DNA methylation degrees at the individual sites, performing a regression analysis in relation to the clinical feature by use of the DNA methylation degree as the variable, and identifying the DNA methylations of which the p value is less than or equal to a fourth threshold in the regression analysis as a first DNA methylation set associated with the clinical feature.

27. The method or device in accordance with embodiment 26, wherein the determining the correlation between the DNA methylation and the clinical feature further comprises determining the risk values of various DNA methylation sites in the first DNA methylation set, wherein the risk values are determined based on the correlation coefficients of the methylation sites obtained in the regression analysis, as well as the methylation degrees of the methylation sites.

28. The method or device in accordance with any one of embodiments 21-27, wherein the at least one biological indicator further comprises the somatic mutation of gene in the patient, and the determining the correlation between the somatic mutation and the clinical feature comprises: determining the somatic mutation contained in the gene in the second gene set, and determining the signal pathway of the gene containing the somatic mutation.

29. The method or device in accordance with any one of embodiments 21-28, wherein the at least one biological indicator comprises the microRNAs in the patient, and the determining the correlation between the microRNA and the clinical feature comprises: determining the microRNA regulating the gene of the second gene set, and determining the correlation between the expression level of the microRNA in the patient and the expression level of gene regulated by the microRNA, identifying the microRNA having a correlation higher than a fifth threshold as a first microRNA set associated with the clinical feature.

30. The method or device in accordance with any one of embodiments 27-29, wherein the determining the correlation between the biological indicator and the clinical feature comprises: determining the weight of the following biological indicators to the clinical feature by performing an ordered logistic regression analysis, respectively: the expression level of genes of the second gene set, the copy number variation of the genes of the second gene set, the risk values of the DNA methylation sites of the first DNA methylation set.

31. The method or device in accordance with embodiment 30 comprising the weight of the expression levels of the individual protective effective genes and the individual risk effective genes of the second gene set, respectively.

32. A computer readable storage medium having a computer program stored therein, wherein the computer program allows the computer to execute the method according to any one of embodiments 3-31.

33. A device of determining a tumor progression in a subject comprising:

a) an analysis module capable of determining the expression levels of the genes as shown in Table 1 in the subject or a biological sample derived from the subject; and

b) a determination module capable of determining the tumor progression in the subject in accordance with the expression level as measured in a).

34. A device of determining a tumor progression in a subject comprising a computer for determining a tumor progression in a subject, said computer being programmed to executing the steps of:

a) determining the expression levels of one or more genes as shown in Table 1 in the subject or a biological sample derived from the subject; and

b) determining the tumor progression in the subject in accordance with the expression level as measured in a).

35. A method of determining a tumor progression in a subject comprising:

a) determining the expression levels of one or more genes as shown in Table 1 in the subject or a biological sample derived from the subject; and

b) determining the tumor progression in the subject in accordance with the expression level as measured in a).

36. The method or device in accordance with any one of embodiments 33-35, wherein the tumor progression comprises the stages of the tumor and/or the survival rate of the subject.

37. The method or device in accordance with embodiment 36, wherein the tumor stage is selected from the group consisting of: Tumor Stage I, Tumor Stage II, Tumor Stage III, and Tumor Stage IV.

38. The method or device in accordance with any one of embodiments 33-37, wherein the tumor comprises bladder cancer.

39. The method or device in accordance with embodiment 38, wherein the bladder cancer comprises Bladder Urothelial Carcinoma (BLCA).

40. The method or device in accordance with any one of embodiments 33-39, wherein the one or more genes comprise at least one or more protective effective genes as shown in Table 2.

41. The method or device in accordance with any one of embodiments 33-40, wherein the one or more genes comprise at least one or more risk effective genes as shown in Table 3.

42. The method or device in accordance with any one of embodiments 33-41, wherein the one or more genes comprise at least one or more genes as shown in Table 4.

43. The method or device in accordance with any one of embodiments 33-42, wherein the one or more genes comprise at least one or more genes as shown in Table 5.

44. The method or device in accordance with any one of embodiments 33-43 further comprising a step or module of determining the copy number variation of the one or more genes.

45. The method or device in accordance with any one of embodiments 33-44 further comprising a step or module of determining the risk values of the DNA methylation of the one or more genes as shown in Table 8.

46. The method or device in accordance with any one of embodiments 33-45 further comprising a step or module of determining the age of the subject.

47. The method or device in accordance with any one of embodiments 33-46, wherein the determining the expression levels of one or more genes as shown in Table 1 in the subject or a biological sample derived from the subject comprises: determining the average expression level of the genes as shown in Table 2 in the one or more genes; and determining the average expression level of the genes as shown in Table 3 in the one or more genes.

48. The method or device in accordance with embodiment 47, comprising determining the tumor progression in the subject in accordance with Formula (I):

ln((P (Stage≤j))/(1-P (Stage≤j)))=Intercept+0.0366*a+0.3386*b+0.3349*c+1.2193*d+0.0084*e−0.048*f (I)

wherein when j=Tumor Stage III, Intercept=0.9609; when j=Tumor Stage I/II, Intercept=−0.6617;

a is the average expression level of the genes as shown in Table 2 in the one or more genes;

b is the average expression level of the genes as shown in Table 3 in the one or more genes;

c is the copy number variation of the one or more genes;

d is the risk value of DNA methylation of the genes as shown in Table 8 in the one or more genes;

e is the subject's age; and

f is the subject's gender, wherein male is 0, and female is 1.

49. A computer readable storage medium having a computer program stored therein, wherein the computer program allows the computer to execute the method according to any one of embodiments 35-48.

50. A method of treating a tumor in a subject comprising:

determining the tumor progression in the subject by use of the method according to any one of embodiments 35-48; and

administering an effective amount of treatment to the subject in accordance with the tumor progression.

51. A device of treating a tumor in a subject comprising:

a) an analysis module capable of determining the expression levels of the one or more genes as shown in Table 1 in the subject or a biological sample derived from the subject;

b) a determination module capable of determining the tumor progression in the subject in accordance with the expression level as measured in a); and

c) a treatment module capable of administering an effective amount of treatment to the subject in accordance with the progression as determined in b).

Without being bound by any theory, the following examples are only for the purpose of illustrating the working mechanism of the device, method, and system of the present application, but are not intended to limit the scope of the invention as claimed in the present application.

### EXAMPLES p All statistical analyses in the examples of the present application were performed by R software (version 3.3.3).

### Example 1

### Data Sources of Patients and Tumor Samples

Most of the genomes of the BLCA patients and the clinical data set as used in the present application were downloaded from “NCI GDC Data Portal Legacy Archive”. Of those, the clinical information of the BLCA patients was from the TCGA-BLCA clinical documents. The obtained RNA-seq data set of the BLCA patients comprised 419 samples, including 400 tumor samples and 19 normal samples. All the expression of genes was normalized.

Somatic mutation data for TCGA level 2 were used in the mutation annotation format (MAF file). Methylation data for TCGA Level 3 were downloaded from “jhu-usc_BLCA. HumanMethylation450”. Correlation data between mRNA expression and DNA methylation for TCGA level 4 were from the Broad GDAC Firehose. Copy number variation (CNV) data for TCGA Level 4 were downloaded from Broad GDAC Firehose.

The following discrete indexes were used to indicate the level of amplification and deletion of CNVs: severe deletion=−2; deletion=1; no change=0; amplification=1; high level amplification=2.

The “per million miRNA mapped (RPM)” from the quantitative files for the TCGA Level 3 microRNA was selected as the microRNA expression.

A list of known miRNA-gene interactions that have been validated by the literature was obtained from miRWalk 2.0. The microRNA-cancer relationship information comes from miRCancer.

### Example 2

### Screening of Key Genes Based on Survival Analysis

The relationship between the survival status and various potential influencing factors (e.g., key genes) was studied by means of survival analysis.

Test Method:

Cox Proportional Hazard Regression

Key genes that are likely to affect the survival of the BLCA patients were identified by use of single- and multi-variable Cox proportional hazard regression model. First, the expression of the individual genes of all the BLCA samples was normalized in accordance with the respective z-scores. And the genes that were merely expressed in less than 20 samples were removed.

In the single-variable Cox proportional hazard regression, the expression of gene was used as the only predictive variable; while in the multi-variable Cox proportional hazard regression, the age, the gender, the tumor stage, and the expression of gene were all used as predictive variables. The “Benjamini & Hochberg” method was used to adjust the p value.

As for the statistically significant thresholds of survival analysis, the p-value of the single-variable Cox proportional hazard regression was <0.05 and the false discovery rate (FDR) was <0.1; and the p-value of the multi-variable Cox proportional hazard regression was <0.05 and the FDR<0.05. For all the Cox regression models, the proportional hazard hypothesis was also examined and those genes that did not meet this hypothesis were removed.

Kaplan-Meier Analysis

For the Kaplan-Meier survival analysis, all the BLCA samples were first divided into high and low groups in accordance with the median of the individual genes as selected. Next, a Kaplan-Meier survival graph was plotted, and the two groups were compared for their difference by running a log-rank test. The survival analysis was performed by use of the R package “survival”.

GO Analysis

The functional annotation of the screened genes and the enrichment analysis of their gene ontology (GO) were performed in DAVID v6.8. The GO function was selected by use of a threshold of the p value <0.05.

Test Results:

A group of key genes which were likely to significantly affect the survival of the BLCA patients were selected by use of the single- and multi-variable Cox proportional hazard regression models. Of those, for the single-variable Cox regression, the expression of gene was used as the only predicator variable. Initially, after removing the genes which were rarely expressed (the genes which were merely expressed in less than 20 samples), the expression of 19472 genes were obtained for all the 404 BLCA patients. Then, 1307 candidate genes were selected based on a threshold of the p value <0.05 and the FDR<0.1. Next, it was examined whether the candidate genes met the proportional hazard (PH) hypothesis, and 99 genes which did not meet the hypothesis were excluded. Thus, 1208 candidate genes were screened by the single-variable Cox regression analysis.

In the multi-variable Cox regression, in addition to the expression of the aforesaid 1208 genes, the information including the age, gender and tumor stage (wherein Stage I/II=3, Stage III=2, Stage IV =1) of the BLCA patients were used as the input predicator variables. The FDR threshold <0.05 was used, and it was examined whether the candidate genes met the proportional hazard (PH) hypothesis for further screening the candidate genes. Finally, 1078 candidate genes were obtained by the multi-variable Cox regression (see, Table 1, where Table 1 showed the identified 1078 key genes), and the 1078 genes as shown in Table 1 were defined as key genes for subsequent analysis.

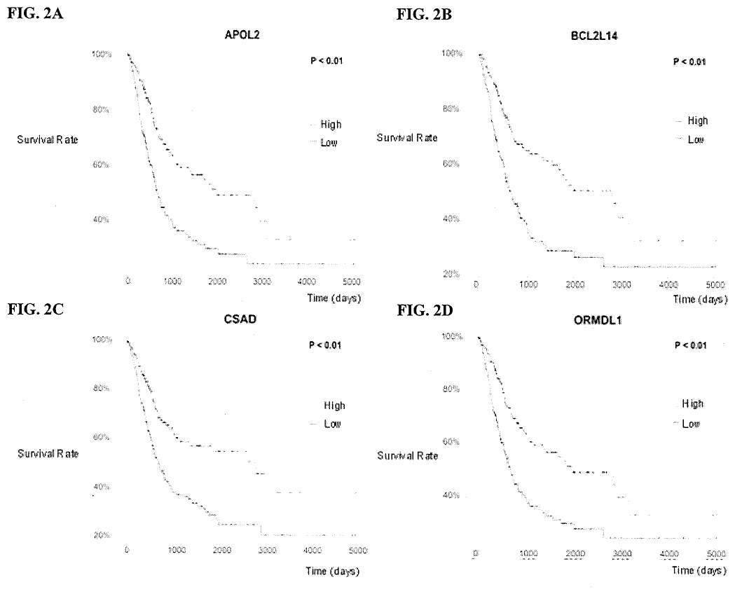

According to the coefficients of gene expression obtained by the aforesaid multi-variable Cox regression model, the 1078 key genes were divided into two groups, wherein 356 genes had negative correlation coefficient values, and 722 genes had positive correlation coefficient value, which were defined as protective effective genes and risk effective genes, respectively (see, Table 2 and Table 3). The Kaplan-Meier graphs as shown in FIGS. 2A-2D utilized four samples as examples, and showed the effect of the screened key genes on the survival of the BLCA patients. FIGS. 2A-2D showed the results of genes APOL2, BCL2L14, CSAD and ORMDL1 in sequence, wherein a log-rank test was used to detect the statistically significant differences.

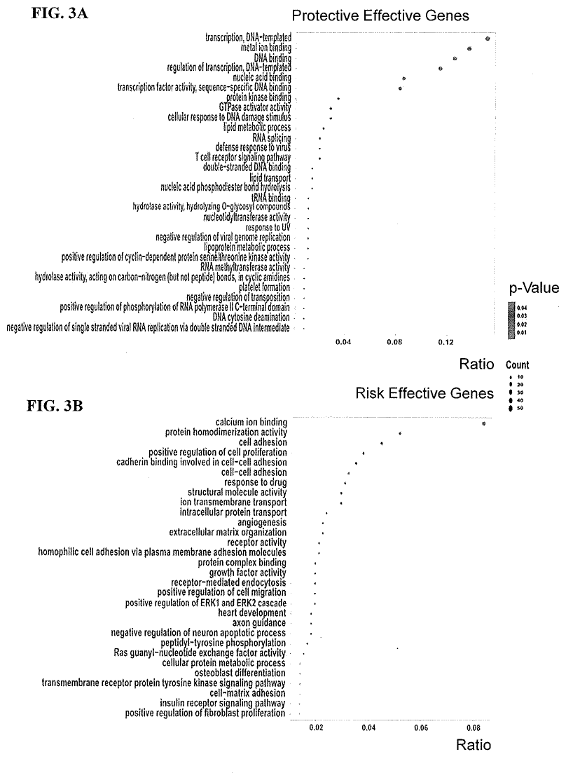

For characterizing the potential biological functions of the key genes as screened above, the above-described protective and risk effective genes were subject to gene ontology (GO) enrichment analysis. As a result, it was found that the GO functions of the protective effective genes lied primarily in the essential cellular processes or functions, such as, nucleic acid binding, RNA splicing, and tRNA binding (see, FIG. 3A). In contrast, the risk effective genes might be involved in the pathogenesis of bladder cancer, such as, cell adhesion, angiogenesis, drug reaction, and positive regulation of cell migration (see, FIG. 3B). The GO functions were ranked in according to the proportion of the involved genes, and FIG. 3 revealed 30 significant GO functions with p values <0.05. In summary, the results of the function enrichment analyses indicated that the screened 1078 key genes, especially those harmful genes, were closely correlated with the biological functions of bladder cancer.

TABLE 1
1078 Key Genes

| | |
|---|---|
| Protective | ACOT13\|55856; AGAP4\|119016; AGAP6\|414189; AGER\|177; AGXT2L2\|85007; AHSA2\|130872; |
| effective | AK3\|50808; ALS2CL\|259173; ANAPC4\|29945; ANGEL2\|90806; ANKRD10\|55608; |
| genes | ANKRD22\|118932; ANO9\|338440; APLF\|200558; APOBEC3D\|140564; APOBEC3F\|200316; |
| | APOBEC3G\|60489; APOL1\|8542; APOL2\|23780; APOL4\|80832; APOL6\|80830; ARRDC2\|27106; |
| | ASB13\|79754; ATF7IP2\|80063; ATOH8\|84913; BAT1\|7919; BATF\|10538; BCL2L14\|79370; |
| | BLOC1S3\|388552; BTN2A1\|11120; C11orf66\|220004; C15orf58\|390637; C17orf86\|654434; |
| | C19orf6\|91304; C19orf66\|55337; C19orf71\|100128569; C1orf126\|200197; C1orf159\|54991; |
| | C1orf213\|148898; C1orf63\|57035; C20orf196\|149840; C20orf96\|140680; C22orf43\|51233; |
| | C2orf60\|129450; C2orf63\|130162; C3orf19\|51244; C3orf23\|285343; C3orf62\|375341; C4orf21\|55345; |
| | C5orf56\|441108; C6orf115\|58527; C6orf134\|79969; C6orf136\|221545; C6orf47\|57827; C6orf62\|81688; |
| | C8orf44\|56260; CARD11\|84433; CASP9\|842; CCDC130\|81576; CCDC14\|64770; CCDC24\|149473; |
| | CCNJL\|79616; CCNL1\|57018; CCNL2\|81669; CCNT2\|905; CCRL2\|9034; CCT6P1\|643253; |
| | CD96\|10225; CDC42EP5\|148170; CDK10\|8558; CDK3\|1018; CELF6\|60677; CHKB-CPT1B\|386593; |
| | CHMP4C\|92421; CIR1\|9541; CLDN15\|24146; CLEC2D\|29121; CLK1\|1195; CNKSR1\|10256; |
| | COLQ\|8292; COX7B\|1349; CPT1B\|1375; CRB3\|92359; CROCCL1\|84809; CRTC2\|200186; |
| | CSAD\|51380; CTRL\|1506; CTU1\|90353; CYorf15B\|84663; CYP2C8\|1558; CYP4Z1\|199974; |
| | CYTH2\|9266; DCAF4L1\|285429; DCDC2B\|149069; DEDD2\|162989; DEGS2\|123099; DMTF1\|9988; |
| | DNAH1\|25981; DNASE1L2\|1775; DOK7\|285489; DOM3Z\|1797; ECHDC2\|55268; EFHD2\|79180; |
| | ELF4\|2000; ELMOD3\|84173; ENGASE\|64772; ERCC5\|2073; ETV7\|51513; FAAH\|2166; |
| | FAAH2\|158584; FAM113B\|91523; FAM122B\|159090; FAM13A\|10144; FAM166B\|730112; |
| | FAM193B\|54540; FAM200B\|285550; FAM73B\|84895; FANCF\|2188; FBP1\|2203; FBXO46\|23403; |
| | FBXO6\|26270; FCHSD1\|89848; FER1L4\|80307; FITM1\|161247; FLJ12825\|440101; FNBP4\|23360; |
| | FOXD4L1\|200350; GAK\|2580; GBA2\|57704; GEMIN8\|54960; GGA1\|26088; GK\|2710; |
| | GLTSCR1\|29998; GLYCTK\|132158; GMIP\|51291; GOLGA2B\|55592; GRIPAP1\|56850; |
| | GSDMB\|55876; HCG26\|352961; HCG27\|253018; HCG4P6\|80868; HDAC10\|83933; HDHD3\|81932; |
| | HEXDC\|284004; HIP1R\|9026; HIST1H2BL\|8340; HIST1H4C\|8364; HIST1H4J\|8363; |
| | HIST2H2AC\|8338; HLA-L\|3139; HMGN4\|10473; HNMT\|3176; HOXB5\|3215; HOXB7\|3217; |
| | HSH2D\|84941; HSPA1L\|3305; ID2B\|84099; IDUA\|3425; IFT27\|11020; IKBKB\|3551; INSL3\|3640; |
| | IP6K2\|51447; IRF1\|3659; KCNJ15\|3772; KIAA0907\|22889; KIAA1530\|57654; KIAA1875\|340390; |
| | KIF21B\|23046; KLHL36\|79786; KLRA1\|10748; LENG8\|114823; LIME1\|54923; LIPT1\|51601; |
| | LMBR1L\|55716; LOC100129637\|100129637; LOC100144604\|100144604; |
| | LOC100272146\|100272146; LOC100286793\|100286793; LOC100288778\|100288778; |
| | LOC146880\|146880; LOC221442\|221442; LOC283314\|283314; LOC284232\|284232; |
| | LOC284233\|284233; LOC284900\|284900; LOC285074\|285074; LOC285359\|285359; |
| | LOC388692\|388692; LOC391322\|391322; LOC400927\|400927; LOC401052\|401052; |
| | LOC440944\|440944; LOC642846\|642846; LOC91316\|91316; LUC7L\|55692; LY6G5B\|58496; |
| | MAPK8IP3\|23162; ME3\|10873; MFAP3L\|9848; MFSD2A\|84879; MID1IP1\|58526; |
| | MRFAP1L1\|114932; MRPS6\|64968; MSL3\|10943; MST1R\|4486; MTERFD3\|80298; MZF1\|7593; |
| | NADSYN1\|55191; NBR2\|10230; NCRNA00105\|80161; NCRNA00115\|79854; NDOR1\|27158; |
| | NFKBID\|84807; NFYA\|4800; NPAS2\|4862; NPIPL3\|23117; NR2F6\|2063; NSUN5P1\|155400; |
| | NSUN5P2\|260294; NSUN6\|221078; NUDT16P1\|152195; NUDT19\|390916; OAS1\|4938; OFD1\|8481; |
| | ORMDL1\|94101; ORMDL3\|94103; P2RY11\|5032; PAQR6\|79957; PAR1\|145624; PARP4\|143; |
| | PATL2\|197135; PBOV1\|59351; PCF11\|51585; PDCL3\|79031; PDXDC2\|283970; PGPEP1\|54858; |
| | PIGA\|5277; PION\|54103; PLA2G6\|8398; PLEKHA6\|22874; PLEKHH1\|57475; PLGLB2\|5342; |
| | PLIN5\|440503; PLXNB1\|5364; PMS1\|5378; PMS2L3\|5387; POLB\|5423; PPFIBP2\|8495; |
| | PRICKLE3\|4007; PRKD2\|25865; PSMB10\|5699; PSMB8\|5696; PTPN6\|5777; PYROXD2\|84795; |
| | RAB28\|9364; RABL2A\|11159; RAD9A\|5883; RBCK1\|10616; RBM6\|10180; REV1\|51455; |
| | RG9MTD3\|158234; RGPD6\|729540; RPL32P3\|132241; RPP21\|79897; RTP2\|344892; RTP4\|64108; |
| | RWDD3\|25950; SCXB\|642658; SDCBP2\|27111; SEC31B\|25956; SEMA4D\|10507; SEPT7P2\|641977; |
| | SERINC4\|619189; SETMAR\|6419; SFRS16\|11129; SFRS17A\|8227; SH3GLB2\|56904; SHC3\|53358; |
| | SKINTL\|391037; SLC10A5\|347051; SLC25A34\|284723; SLC45A3\|85414; SLC5A9\|200010; |
| | SLC7A9\|11136; SMAD6\|4091; SP140L\|93349; SPDYA\|245711; SPG7\|6687; SPOCD1\|90853; |
| | SPSB3\|90864; STAG3L3\|442578; STAP2\|55620; STAT6\|6778; SYCP3\|50511; TAF1C\|9013; |
| | TBC1D3\|729873; TBC1D3B\|414059; TBC1D3P2\|440452; TCEANC\|170082; TCTE3\|6991; |
| | THUMPD2\|80745; TIA1\|7072; TMC7\|79905; TMEM51\|55092; TNFAIP2\|7127; TNK1\|8711; |
| | TOP3B\|8940; TRAPPC2\|6399; TRIM26\|7726; TRIM27\|5987; TRIM38\|10475; TRPV1\|7442; |
| | TSPAN14\|81619; TTLL3\|26140; UBD\|10537; UCP3\|7352; UNC93B1\|81622; USF1\|7391; |
| | WASH3P\|374666; WASH7P\|653635; WDR52\|55779; WDR6\|11180; YTHDC1\|91746; |
| | ZCWPW1\|55063; ZFPM1\|161882; ZNF100\|163227; ZNF137\|7696; ZNF160\|90338; ZNF165\|7718; |
| | ZNF169\|169841; ZNF182\|7569; ZNF187\|7741; ZNF193\|7746; ZNF195\|7748; ZNF254\|9534; |
| | ZNF443\|10224; ZNF480\|147657; ZNF493\|284443; ZNF506\|440515; ZNF513\|130557; ZNF524\|147807; |
| | ZNF564\|163050; ZNF577\|84765; ZNF600\|162966; ZNF630\|57232; ZNF638\|27332; ZNF708\|7562; |
| | ZNF763\|284390; ZNF799\|90576; ZNF814\|730051; ZNF823\|55552; ZNF841\|284371; ZNRD1\|30834; |
| | ZRANB2\|9406; ZRSR2\|8233; ZSCAN16\|80345 |
| Risk | ABCB9\|23457; ABCC1\|4363; ABCC9\|10060; ABCE1\|6059; ACCN1\|40; ACCN2\|41; ACLY\|47; |
| effective | ACVR1\|90; ADAM23\|8745; ADAMTS12\|81792; ADAMTS16\|170690; ADAMTS18\|170692; |
| genes | ADAMTSL1\|92949; ADCY7\|113; ADRA1B\|147; ADRA1D\|146; ADRA2B\|151; AHCY\|191; |
| | AHNAK\|79026; AIF1L\|83543; AKR1B1\|231; AKR1B15\|441282; AKR7A2\|8574; ALAS2\|212; |
| | ALDH1L2\|160428; ALG1\|56052; ALPL\|249; AMDHD1\|144193; ANGPT1\|284; ANLN\|54443; |
| | ANO1\|55107; ANPEP\|290; ANXA1\|301; ANXA2\|302; ANXA2P1\|303; ANXA2P2\|304; ANXA5\|308; |
| | AP2A2\|161; AP2B1\|163; ARCN1\|372; ARHGAP29\|9411; ARID3A\|1820; ARL10\|285598; |
| | ARL4C\|10123; ARMC9\|80210; ARSI\|340075; ASPM\|259266; ATF6\|22926; ATG9A\|79065; |
| | ATP12A\|479; ATP13A4\|84239; ATP1A1\|476; ATP2B4\|493; ATP6V0A1\|535; ATP6V0D1\|9114; |
| | ATP6V1A\|523; ATP6V1B1\|525; ATP6V1B2\|526; ATP6V1C2\|245973; ATP8B2\|57198; AVIL\|10677; |
| | AXIN2\|8313; B3GALT2\|8707; B4GALNT1\|2583; B4GALNT2\|124872; BACE1\|23621; |
| | BAIAP2\|10458; BARX2\|8538; BRMS1L\|84312; BSND\|7809; C10orf90\|118611; C11orf16\|56673; |
| | C11orf20\|25858; C11orf53\|341032; C11orf87\|399947; C12orf61\|283416; C13orf15\|28984; |
| | C13orf33\|84935; C13orf39\|196541; C14orf126\| 112487; C14orf128\|84837; C14orf37\|145407: |
| | C14orf86\|283592; C15orf38\|348110; C15orf54\|400360; C16orf63\|123811; C17orf39\|79018: |
| | C17orf51\|339263; C18orf20\|221241; C18orf22\|79863; C18orf54\|162681; C19orf26\|255057; |
| | C19orf59\|199675; C1orf84\|149469; C20orf117\|140710; C20orf177\|63939; C2orf62\|375307; |
| | C5orf13\|9315; C5orf62\|85027; C6orf138\|442213; C6orf72\|116254; C7orf33\|202865; C8orf31\|286122; |
| | C9orf24\|84688; CA10\|56934; CA5A\|763; CA7\|766; CACNA1B\|774; CAD\|790; CALCA\|796; |
| | CALHM3\|119395; CALM1\|801; CALML3\|810; CALU\|813; CAPG\|822; CAPN2\|824; CARD9\|64170; |
| | CAST\|831; CBLN4\|140689; CCDC102B\|79839; CCDC21\|64793; CCDC54\|84692; CCDC8\|83987; |
| | CCDC80\|151887; CCNA1\|8900; CCT6A\|908; CD109\|135228; CD276\|80381; CD300LG\|146894; |
| | CDC73\|79577; CDK6\|1021; CDKN1C\|1028; CEACAM16\|388551; CEACAM21\|90273; |
| | CELA3B\|23436; CERCAM\|51148; CERK\|64781; CES4\|51716; CGA\|1081; CGB1\|114335; |
| | CHPF2\|54480; CHRNA1\|1134; CHSY1\|22856; CIDEC\|63924; CKAP4\|10970; CKMT2\|1160; |
| | CLDN10\|9071; CLEC11A\|6320; CLEC4G\|339390; CLIC3\|9022; CLSTN2\|64084; CLTC\|1213; |
| | CMTM2\|146225; CNIH\|10175; CNN3\|1266; CNTN1\|1272; CNTNAP3\|79937; COBL\|23242; |
| | COG8\|84342; COL18A1\|80781; COL4A2\|1284; COL5A1\|1289; COL5A3\|50509; COL6A1\|1291; |
| | COL9A3\|1299; COPS3\|8533; COPS8\|10920; COPZ2\|51226; COX8C\|341947; CPXM1\|56265; |
| | CRNN\|49860; CRTAP\|10491; CSF3R\|1441; CSGALNACT2\|55454; CSNK1A1P\|161635; CSPG4\|1464; |
| | CTNNB1\|1499; CTPS\|1503; CTRB1\|1504; CTRB2\|440387; CUBN\|8029; CXADRP2\|646243; |
| | CXCL12\|6387; CXCR1\|3577; CXCR7\|57007; CYP19A1\|1588; CYTH3\|9265; CYTL1\|54360; |
| | DAD1\|1603; DARS2\|55157; DBN1\|1627; DDB1\|1642; DDX10\|1662; DDX21\|9188; DIRAS3\|9077; |
| | DISP2\|85455; DLX1\|1745; DLX4\|1748; DMRT3\|58524; DNAJB4\|11080; DNM3\|26052; |
| | DNMT3L\|29947; DNTT\|1791; DPH3B\|100132911; DSC1\|1823; DSEL\|92126; DSTYK\|25778; |
| | DUSP13\|51207; DUSP14\|11072; DYM\|54808; DYRK3\|8444; ECM1\|1893; EDNRA\|1909; |
| | EFCAB1\|79645; EHBP1\|23301; EIF2AK4\|440275; EIF3A\|8661; EIF4A3\|9775; EIF4E1B\|253314; |
| | ELOVL4\|6785; EMP1\|2012; EMP3\|2014; ENDOD1\|23052; ENKUR\|219670; ENPP1\|5167; |
| | ENTPD2\|954; EPDR1\|54749; EPHB3\|2049; EPHB4\|2050; EPN2\|22905; EPRS\|2058; ERC1\|23085; |
| | ERMN\|57471; ESD\|2098; ESF1\|51575; ESYT2\|57488; ETF1\|2107; EVC2\|132884; EXTL3\|2137; |
| | EYS\|346007; F10\|2159; F13A1\|2162; F2RL2\|2151; FAM101B\|359845; FAM110B\|90362; |
| | FAM126A\|84668; FAM129B\|64855; FAM168A\|23201; FAM180A\|389558; FAM20C\|56975; |
| | FAM25A\|643161; FAM25B\|100132929; FAM27B\|100133121; FAM43A\|131583; FAM49A\|81553; |
| | FAM5C\|339479; FASN\|2194; FGF1\|2246; FGF12\|2257; FGF19\|9965; FHL3\|2275; FJX1\|24147; |
| | FKBP10\|60681; FKBP14\|55033; FKBP9\|11328; FLJ42709\|441094; FLJ43390\|646113; FLRT2\|23768; |
| | FN1\|2335; FNTB\|2342; FOLR2\|2350; FOXI1\|2299; FOXL1\|2300; FRG2\|448831; FRG2B\|441581; |
| | FUT11\|170384; G6PD\|2539; GABRA3\|2556; GABRG1\|2565; GALK1\|2584; GANAB\|23193; |
| | GAS7\|8522; GBX2\|2637; GCG\|2641; GEMIN5\|25929; GFPT2\|9945; GGTLC1\|92086; GJA1\|2697; |
| | GLCE\|26035; GLI2\|2736; GLT25D1\|79709; GNA12\|2768; GOLGA8G\|283768; GPC1\|2817; |
| | GPHN\|10243; GPR32\|2854; GPR37\|2861; GPSM2\|29899; GPX8\|493869; GRAMD2\|196996; |
| | GRB14\|2888; GRIK2\|2898; GRK5\|2869; GTF2A1\|2957; GUCA1A\|2978; GUCY1B3\|2983; |
| | GULP1\|51454; GXYLT2\|727936; HAUS2\|55142; HDAC4\|9759; HDAC5\|10014; HDLBP\|3069; |
| | HECW1\|23072; HEPACAM2\|253012; HEYL\|26508; HHIPL2\|79802; HIPK2\|28996; HOXC5\|3222; |
| | HOXC8\|3224; HPD\|3242; HSPA6\|3310; HTRA4\|203100; IARS2\|55699; ICAM5\|7087; IFT122\|55764; |
| | IGF1\|3479; IGF2BP3\|10643; IGF2R\|3482; IGFL2\|147920; IL12A\|3592; IL31RA\|133396; |
| | IMPDH1\|3614; INHBB\|3625; INS\|3630; INSRR\|3645; IPO11\|51194; IPO4\|79711; IQGAP1\|8826; |
| | ITFG1\|81533; ITGA1\|3672; ITGB8\|3696; JAG1\|182; JDP2\|122953; KANK4\|163782; KCNE4\|23704; |
| | KCNH2\|3757; KCNU1\|157855; KCTD20\|222658; KCTD4\|386618; KDELC2\|143888; KDSR\|2531; |
| | KIAA0087\|9808; KIAA0090\|23065; KIAA0391\|9692; KIAA1328\|57536; KIAA1598\|57698; |
| | KIAA1919\|91749; KIF1B\|23095; KIF25\|3834; KIF26A\|26153; KIFAP3\|22920; KLHDC10\|23008; |
| | KLRG2\|346689; KPNB1\|3837; KRT23\|25984; KRT4\|3851; KRT79\|338785; KRTAP5-2\|440021; |
| | KRTDAP\|388533; L1TD1\|54596; LAMC1\|3915; LCN1\|3933; LDLR\|3949; LDLRAD3\|143458; |
| | LEPROT\|54741; LGALS1\|3956; LGTN\|1939; LHFP\|10186; LIN28A\|79727; LIN28B\|389421; |
| | LMAN1\|3998; LOC100192378\|100192378; LOC100216001\|100216001; LOC151162\|151162; |
| | LOC338588\|338588; LOC441208\|441208; LOX\|4015; LRP1\|4035; LRP12\|29967; LRP1B\|53353; |
| | LTBP1\|4052; LYVE1\|10894; MAFG\|4097; MAGEB16\|139604; MAN2A1\|4124; MAP1A\|4130; |
| | MAP1B\|4131; MAP2\|4133; MAP2K1\|5604; MAP7D1\|55700; MAP7D3\|79649; MAPK3\|5595; |
| | MARVELD1\|83742; MBOAT2\|129642; MDGA2\|161357; ME1\|4199; MED19\|219541; MEP1B\|4225; |
| | MESTIT1\|317751; MFF\|56947; MFSD11\|79157; MGC12916\|84815; MGC4473\|79100; |
| | MGC45800\|90768; MMP16\|4325; MMS19\|64210; MPRIP\|23164; MRO\|83876; MRPL37\|51253; |
| | MT1A\|4489; MTMR2\|8898; MXRA7\|439921; MYADM\|91663; MYH10\|4628; MYO5A\|4644; |
| | MYO9A\|4649; NAMPT\|10135; NAV3\|89795; NBAS\|51594; NCAM1\|4684; NCAPD2\|9918; |
| | NEBL\|10529; NEFL\|4747; NELF\|26012; NELL2\|4753; NES\|10763; NEURL\|9148; NGF\|4803; |
| | NHEDC2\|133308; NID2\|22795; NKX6-2\|84504; NLN\|57486; NLRP12\|91662; NOTCH3\|4854; |
| | NPAS3\|64067; NPC1\|4864; NPHP4\|261734; NPR3\|4883; NR0B1\|190; NRCAM\|4897; NRSN2\|80023; |
| | NT5C3L\|115024; NTRK1\|4914; NTRK2\|4915; NTS\|4922; NUCKS1\|64710; NUDT11\|55190; |
| | NUP188\|23511; NXPH3\|11248; NXPH4\|11247; OBP2A\|29991; ODZ3\|55714; OLFML2B\|25903; |
| | OR2W3\|343171; OSBPL10\|114884; OSCP1\|127700; OTX1\|5013; OTX2\|5015; P4HB\|5034; |
| | PADI4\|23569; PAFAH1B2\|5049; PAM\|5066; PAPPA\|5069; PCDH12\|51294; PCDHB10\|56126; |
| | PCDHB11\|56125; PCDHB12\|56124; PCDHB7\|56129; PCDHB8\|56128; PCDHGA1\|56114; |
| | PCDHGA2\|56113; PCDHGA3\|56112; PCDHGB1\|56104; PCDHGC3\|5098; PCLO\|27445; |
| | PCOLCE2\|26577; PCSK5\|5125; PDE5A\|8654; PDE6H\|5149; PDGFC\|56034; PDGFD\|80310; |
| | PDGFRA\|5156; PDGFRB\|5159; PDIA6\|10130; PDLIM2\|64236; PEG10\|23089; PFKM\|5213; |
| | PGA3\|643834; PGA5\|5222; PGF\|5228; PGLYRP3\|114771; PGLYRP4\|57115; PGM3\|5238; |
| | PHOSPHO1\|162466; PIGS\|94005; PIK3C3\|5289; PINX1\|54984; PIP\|5304; PITX3\|5309; |
| | PIWIL3\|440822; PLA2G1B\|5319; PLAGL1\|5325; PLCZ1\|89869; PLD5\|200150; PLEKHG4B\|153478; |
| | POLR3D\|661; PPEF1\|5475; PPP2R2C\|5522; PPP2R3A\|5523; PPT2\|9374; PPY\|5539; PRDM13\|59336; |
| | PRKAR2A\|5576; PRL\|5617; PRMT5\|10419; PRND\|23627; PRNP\|5621; PROKR2\|128674; |
| | PRPF19\|27339; PRR11\|55771; PRRT4\|401399; PRSS23\|11098; PRSS27\|83886; PRSS37\|136242; |
| | PRSS8\|5652; PTF1A\|256297; PTPLB\|201562; PTPN14\|5784; PTPN21\|11099; PTPRG\|5793; |
| | PVT1\|5820; RAB5C\|5878; RAC3\|5881; RAPGEF5\|9771; RASA1\|5921; RASAL2\|9462; RASD1\|51655; |
| | RASGEF1C\|255426; RASGRP4\|115727; RBBP5\|5929; RBMS3\|27303; RBP7\|116362; RCAN1\|1827; |
| | RDX\|5962; REEP6\|92840; REG1A\|5967; RGS17\|26575; RHOXF2B\|727940; RIMBP2\|23504; |
| | RNF217\|154214; RNF26\|79102; RNF40\|9810; RPAP1\|26015; RPTOR\|57521; RTTN\|25914; |
| | RUNX2\|860; SAMD8\|142891; SARS\|6301; SC65\|10609; SCD\|6319; SCEL\|8796; SCGB2A2\|4250; |
| | SCRN1\|9805; SEC23A\|10484; SEPT7\|989; SERINC1\|57515; SERPINB10\|5273; SERPINB12\|89777; |
| | SERPINF1\|5176; SERPINI1\|5274; SFRP5\|6425; SGCB\|6443; SGTB\|54557; SH2D6\|284948; |
| | SHC4\|399694; SIDT2\|51092; SIGLEC6\|946; SLC12A2\|6558; SLC12A3\|6559; SLC13A5\|284111; |
| | SLC16A6\|9120; SLC16A9\|220963; SLC1A5\|6510; SLC1A6\|6511; SLC22A11\|55867; SLC27A6\|28965; |
| | SLC2A12\|154091; SLC2A14\|144195; SLC2A3\|6515; SLC38A11\|151258; SLC45A1\|50651; |
| | SLC47A1\|55244; SLC6A2\|6530; SLC9A3R1\|9368; SLCO3A1\|28232; SNAI2\|6591; SNX17\|9784; |
| | SNX2\|6643; SNX24\|28966; SORBS3\|10174; SORT1\|6272; SOST\|50964; SOSTDC1\|25928; |
| | SPANXC\|64663; SPNS1\|83985; SPOCK1\|6695; SPRR3\|6707; SPSB4\|92369; SPTBN2\|6712; |
| | SRP54\|6729; SRP68\|6730; SRPX\|8406; SSRP1\|6749; STAC2\|342667; STK32B\|55351; STRAP\|11171; |
| | STRN\|6801; STT3A\|3703; STX5\|6811; STXBP1\|6812; STXBP5L\|9515; STYX\|6815; SULF2\|55959; |
| | SUMF2\|25870; SUN3\|256979; SUPT16H\|11198; SUPT6H\|6830; SUSD2\|56241; SVEP1\|79987; |
| | SYDE1\|85360; TAF13\|6884; TAF4B\|6875; TAS1R3\|83756; TBC1D16\|125058; TBCD\|6904; |
| | TBXA2R\|6915; TBXAS1\|6916; TCF4\|6925; TCL1B\|9623; TEAD4\|7004; TECR\|9524; TET1\|80312; |
| | TEX2\|55852; TGFBI\|7045; TGFBR2\|7048; THBS3\|7059; THOP1\|7064; TKT\|7086; TM4SF1\|4071; |
| | TMCC2\|9911; TMEM104\|54868; TMEM109\|79073; TMEM158\|25907; TMEM17\|200728; |
| | TMEM26\|219623; TMEM48\|55706; TMEM5\|10329; TMEM61\|199964; TMPRSS15\|5651; |
| | TMTC1\|83857; TMX2\|51075; TNFAIP8L3\|388121; TNFRSF6B\|8771; TNN\|63923; TOX4\|9878; |
| | TPD52L1\|7164; TPPP3\|51673; TPST1\|8460; TRAM2\|9697; TREML3\|340206; TREML4\|285852; |
| | TRIM16\|10626; TRIM16L\|147166; TRIM9\|114088; TRIML1\|339976; TROVE2\|6738; TRPV2\|51393; |
| | TSPAN9\|10867; TSPYL6\|388951; TUBA1A\|7846; TUBAL3\|79861; TUBGCP5\|114791; TULP3\|7289; |
| | TWIST2\|117581; TXNRD1\|7296; TYRO3\|7301; UACA\|55075; UBE2QL1\|134111; UBTD1\|80019; |
| | UCHL1\|7345; UCHL5\|51377; UPK3A\|7380; USP13\|8975; USP5\|8078; UTP18\|51096; VDAC2\|7417; |
| | VIM\|7431; VKORC1\|79001; VWA5B2\|90113; WLS\|79971; WWTR1\|25937; XAGE2\|9502; |
| | XPOT\|11260; YARS\|8565; ZC3HAV1L\|92092; ZNF385D\|79750; ZNF474\|133923; ZNF532\|55205; |
| | ZNF705A\|440077; ZNF804B\|219578; ZSCAN5B\|342933; ZW10\|9183 |

TABLE 2
Protective Effective genes

| | |
|---|---|
| Protective | ACOT13\|55856; AGAP4\|119016; AGAP6\|414189; AGER\|177; AGXT2L2\|85007; AHSA2\|130872; |
| effective | AK3\|50808; ALS2CL\|259173; ANAPC4\|29945; ANGEL2\|90806; ANKRD10\|55608; |
| genes | ANKRD22\|118932; ANO9\|338440; APLF\|200558; APOBEC3D\|140564; APOBEC3F\|200316; |
| | APOBEC3G\|60489; APOL1\|8542; APOL2\|23780; APOL4\|80832; APOL6\|80830; ARRDC2\|27106; |
| | ASB13\|79754; ATF7IP2\|80063; ATOH8\|84913; BAT1\|7919; BATF\|10538; BCL2L14\|79370; |
| | BLOC1S3\|388552; BTN2A1\|11120; C11orf66\|220004; C15orf58\|390637; C17orf86\|654434; |
| | C19orf6\|91304; C19orf66\|55337; C19orf71\|100128569; C1orf126\|200197; C1orf159\|54991; |
| | C1orf213\|148898; C1orf63\|57035; C20orf196\|149840; C20orf96\|140680; C22orf43\|51233; |
| | C2orf60\|129450; C2orf63\|130162; C3orf19\|51244; C3orf23\|285343; C3orf62\|375341; C4orf21\|55345; |
| | C5orf56\|441108; C6orf115\|58527; C6orf134\|79969; C6orf136\|221545; C6orf47\|57827; C6orf62\|81688; |
| | C8orf44\|56260; CARD11\|84433; CASP9\|842; CCDC130\|81576; CCDC14\|64770; CCDC24\|149473; |
| | CCNJL\|79616; CCNL1\|57018; CCNL2\|81669; CCNT2\|905; CCRL2\|9034; CCT6P1\|643253; |
| | CD96\|10225; CDC42EP5\|148170; CDK10\|8558; CDK3\|1018; CELF6\|60677; CHKB-CPT1B\|386593; |
| | CHMP4C\|92421; CIR1\|9541; CLDN15\|24146; CLEC2D\|29121; CLK1\|1195; CNKSR1\|10256; |
| | COLQ\|8292; COX7B\|1349; CPT1B\|1375; CRB3\|92359; CROCCL1\|84809; CRTC2\|200186; |
| | CSAD\|51380; CTRL\|1506; CTU1\|90353; CYorf15B\|84663; CYP2C8\|1558; CYP4Z1\|199974; |
| | CYTH2\|9266; DCAF4L1\|285429; DCDC2B\|149069; DEDD2\|162989; DEGS2\|123099; DMTF1\|9988; |
| | DNAH1\|25981; DNASE1L2\|1775; DOK7\|285489; DOM3Z\|1797; ECHDC2\|55268; EFHD2\|79180; |
| | ELF4\|2000; ELMOD3\|84173; ENGASE\|64772; ERCC5\|2073; ETV7\|51513; FAAH\|2166; |
| | FAAH2\|158584; FAM113B\|91523; FAM122B\|159090; FAM13A\|10144; FAM166B\|730112; |
| | FAM193B\|54540; FAM200B\|285550; FAM73B\|84895; FANCF\|2188; FBP1\|2203; FBXO46\|23403; |
| | FBXO6\|26270; FCHSD1\|89848; FER1L4\|80307; FITM1\|161247; FLJ12825\|440101; FNBP4\|23360; |
| | FOXD4L1\|200350; GAK\|2580; GBA2\|57704; GEMIN8\|54960; GGA1\|26088; GK\|2710; |
| | GLTSCR1\|29998; GLYCTK\|132158; GMIP\|51291; GOLGA2B\|55592; GRIPAP1\|56850; |
| | GSDMB\|55876; HCG26\|352961; HCG27\|253018; HCG4P6\|80868; HDAC10\|83933; HDHD3\|81932; |
| | HEXDC\|284004; HIP1R\|9026; HIST1H2BL\|8340; HIST1H4C\|8364; HIST1H4J\|8363; |
| | HIST2H2AC\|8338; HLA-L\|3139; HMGN4\|10473; HNMT\|3176; HOXB5\|3215; HOXB7\|3217; |
| | HSH2D\|84941; HSPA1L\|3305; ID2B\|84099; IDUA\|3425; IFT27\|11020; IKBKB\|3551; INSL3\|3640; |
| | IP6K2\|51447; IRF1\|3659; KCNJ15\|3772; KIAA0907\|22889; KIAA1530\|57654; KIAA1875\|340390; |
| | KIF21B\|23046; KLHL36\|79786; KLRA1\|10748; LENG8\|114823; LIME1\|54923; LIPT1\|51601; |
| | LMBR1L\|55716; LOC100129637\|100129637; LOC100144604\|100144604; |
| | LOC100272146\|100272146; LOC100286793\|100286793; LOC100288778\|100288778; |
| | LOC146880\|146880; LOC221442\|221442; LOC283314\|283314; LOC284232\|284232; |
| | LOC284233\|284233; LOC284900\|284900; LOC285074\|285074; LOC285359\|285359; |
| | LOC388692\|388692; LOC391322\|391322; LOC400927\|400927; LOC401052\|401052; |
| | LOC440944\|440944; LOC642846\|642846; LOC91316\|91316; LUC7L\|55692; LY6G5B\|58496; |
| | MAPK8IP3\|23162; ME3\|10873; MFAP3L\|9848; MFSD2A\|84879; MID1IP1\|58526; |
| | MRFAP1L1\|114932; MRPS6\|64968; MSL3\|10943; MST1R\|4486; MTERFD3\|80298; MZF1\|7593; |
| | NADSYN1\|55191; NBR2\|10230; NCRNA00105\|80161; NCRNA00115\|79854; NDOR1\|27158; |
| | NFKBID\|84807; NFYA\|4800; NPAS2\|4862; NPIPL3\|23117; NR2F6\|2063; NSUN5P1\|155400; |
| | NSUN5P2\|260294; NSUN6\|221078; NUDT16P1\|152195; NUDT19\|390916; OAS1\|4938; OFD1\|8481; |
| | ORMDL1\|94101; ORMDL3\|94103; P2RY11\|5O32; PAQR6\|79957; PAR1\|145624; PARP4\|143; |
| | PATL2\|197135; PBOV1\|59351; PCF11\|51585; PDCL3\|79031; PDXDC2\|283970; PGPEP1\|54858; |
| | PIGA\|5277; PION\|54103; PLA2G6\|8398; PLEKHA6\|22874; PLEKHH1\|57475; PLGLB2\|5342; |
| | PLIN5\|440503; PLXNB1\|5364; PMS1\|5378; PMS2L3\|5387; POLB\|5423; PPFIBP2\|8495; |
| | PRICKLE3\|4007; PRKD2\|25865; PSMB10\|5699; PSMB8\|5696; PTPN6\|5777; PYROXD2\|84795; |
| | RAB28\|9364; RABL2A\|11159; RAD9A\|5883; RBCK1\|10616; RBM6\|10180; REV1\|51455; |
| | RG9MTD3\|158234; RGPD6\|729540; RPL32P3\|132241; RPP21\|79897; RTP2\|344892; RTP4\|64108; |
| | RWDD3\|25950; SCXB\|642658; SDCBP2\|27111; SEC31B\|25956; SEMA4D\|10507; SEPT7P2\|641977; |
| | SERINC4\|619189; SETMAR\|6419; SFRS16\|11129; SFRS17A\|8227; SH3GLB2\|56904; SHC3\|53358; |
| | SKINTL\|391037; SLC10A5\|347051; SLC25A34\|284723; SLC45A3\|85414; SLC5A9\|200010; |
| | SLC7A9\|11136; SMAD6\|4091; SP140L\|93349; SPDYA\|245711; SPG7\|6687; SPOCD1\|90853; |
| | SPSB3\|90864; STAG3L3\|442578; STAP2\|55620; STAT6\|6778; SYCP3\|50511; TAF1C,\|9013; |
| | TBC1D3\|729873; TBC1D3B\|414059; TBC1D3P2\|440452; TCEANC\|170082; TCTE3\|6991; |
| | THUMPD2\|80745; TIA1\|7072; TMC7\|79905; TMEM51\|55092; TNFAIP2\|7127; TNK1\|8711; |
| | TOP3B\|8940; TRAPPC2\|6399; TRIM26\|7726; TRIM27\|5987; TRIM38\|10475; TRPV1\|7442; |
| | TSPAN14\|81619; TTLL3\|26140; UBD\|10537; UCP3\|7352; UNC93B1\|81622; USF1\|7391; |
| | WASH3P\|374666; WASH7P\|653635; WDR52\|55779; WDR6\|11180; YTHDC1\|91746; |
| | ZCWPW1\|55063; ZFPM1\|161882; ZNF100\|163227; ZNF137\|7696; ZNF160\|90338; ZNF165\|7718; |
| | ZNF169\|169841; ZNF182\|7569; ZNF187\|7741; ZNF193\|7746; ZNF195\|7748; ZNF254\|9534; |
| | ZNF443\|10224; ZNF480\|147657; ZNF493\|284443; ZNF506\|440515; ZNF513\|130557; ZNF524\|147807; |
| | ZNF564\|163050; ZNF577\|84765; ZNF600\|162966; ZNF630\|57232; ZNF638\|27332; ZNF708\|7562; |
| | ZNF763\|284390; ZNF799\|90576; ZNF814\|730051; ZNF823\|55552; ZNF841\|284371; ZNRD1\|30834; |
| | ZRANB2\|9406; ZRSR2\|8233; ZSCAN16\|80345 |

TABLE 3
Risk Effective genes

| | |
|---|---|
| Risk | ABCB9\|23457; ABCC1\|4363; ABCC9\|10060; ABCE1\|6059; ACCN1\|40; ACCN2\|41; ACLY\|47; |
| effective | ACVR1\|90; ADAM23\|8745; ADAMTS12\|81792; ADAMTS16\|170690; ADAMTS18\|170692; |
| genes | ADAMTSL1\|92949; ADCY7\|113; ADRA1B\|147; ADRA1D\|146; ADRA2B\|151; AHCY\|191; |
| | AHNAK\|79026; AIF1L\|83543; AKR1B1\|231; AKR1B15\|441282; AKR7A2\|8574; ALAS2\|212; |
| | ALDH1L2\|160428; ALG1\|56052; ALPL\|249; AMDHD1\|144193; ANGPT1\|284; ANLN\|54443; |
| | ANO1\|55107; ANPEP\|290; ANXA1\|301; ANXA2\|302; ANXA2P1\|303; ANXA2P2\|304; ANXA5\|308; |
| | AP2A2\|161; AP2B1\|163; ARCN1\|372; ARHGAP29\|9411; ARID3A\|1820; ARL10\|285598; |
| | ARL4C\|10123; ARMC9\|80210; ARSI\|340075; ASPM\|259266; ATF6\|22926; ATG9A\|79065; |
| | ATP12A\|479; ATP13A4\|84239; ATP1A1\|476; ATP2B4\|493; ATP6V0A1\|535; ATP6V0D1\|9114; |
| | ATP6V1A\|523; ATP6V1B1\|525; ATP6V1B2\|526; ATP6V1C2\|245973; ATP8B2\|57198; AVIL\|10677; |
| | AXIN2\|8313; B3GALT2\|8707; B4GALNT1\|2583; B4GALNT2\|124872; BACE1\|23621; BAIAP2\|10458; |
| | BARX2\|8538; BRMS1L\|84312; BSND\|7809; C10orf90\|118611; C11orf16\|56673; C11orf20\|25858; |
| | C11orf53\|341032; C11orf87\|399947; C12orf61\|283416; C13orf15\|28984; C13orf33\|84935; |
| | C13orf39\|196541; C14orf126\|112487; C14orf128\|84837; C14orf37\|145407; C14orf86\|283592; |
| | C15orf38\|348110; C15orf54\|400360; C16orf63\|123811; C17orf39\|79018; C17orf51\|339263; |
| | C18orf20 221241; C18orf22\|79863; C18orf54\|162681; C19orf26\|255057; C19orf59\|199675; |
| | C1orf84\|149469; C20orf117\|140710; C20orf177\|63939; C2orf62\|375307; C5orf13\|9315; C5orf62\|85027; |
| | C6orf138\|442213; C6orf72\|116254; C7orf33\|202865; C8orf31\|286122; C9orf24\|84688; CA10\|56934; |
| | CA5A\|763; CA7\|766; CACNA1B\|774; CAD\|790; CALCA\|796; CALHM3\|119395; CALM1\|801; |
| | CALML3\|810; CALU\|813; CAPG\|822; CAPN2\|824; CARD9\|64170; CAST\|831; CBLN4\|140689; |
| | CCDC102B\|79839; CCDC21\|64793; CCDC54\|84692; CCDC8\|83987; CCDC80\|151887; CCNA1\|8900; |
| | CCT6A\|908; CD109\|135228; CD276\|80381; CD300LG\|146894; CDC73\|79577; CDK6\|1021; |
| | CDKN1C\|1028; CEACAM16\|388551; CEACAM21\|90273; CELA3B\|23436; CERCAM\|51148; |
| | CERK\|64781; CES4\|51716; CGA\|1081; CGB1\|114335; CHPF2\|54480; CHRNA1\|1134; CHSY1\|22856; |
| | CIDEC\|63924; CKAP4\|10970; CKMT2\|1160; CLDN10\|9071; CLEC11A\|6320; CLEC4G\|339390; |
| | CLIC3\|9022; CLSTN2\|64084; CLTC\|1213; CMTM2\|146225; CNIH\|10175; CNN3\|1266; CNTN1\|1272; |
| | CNTNAP3\|79937; COBL\|23242; COG8\|84342; COL18A1\|80781; COL4A2\|1284; COL5A1\|1289; |
| | COL5A3\|50509; COL6A1\|1291; COL9A3\|1299; COPS3\|8533; COPS8\|10920; COPZ2\|51226; |
| | COX8C\|341947; CPXM1\|56265; CRNN\|49860; CRTAP\|10491; CSF3R\|1441; CSGALNACT2\|55454; |
| | CSNK1A1P\|161635; CSPG4\|1464; CTNNB1\|1499; CTPS\|1503; CTRB1\|1504; CTRB2\|440387; |
| | CUBN\|8029; CXADRP2\|646243; CXCL12\|6387; CXCR1\|3577; CXCR7\|57007; CYP19A1\|1588; |
| | CYTH3\|9265; CYTL1\|54360; DAD1\|1603; DARS2\|55157; DBN1\|1627; DDB1\|1642; DDX10\|1662; |
| | DDX21\|9188; DIRAS3\|9077; DISP2\|85455; DLX1\|1745; DLX4\|1748; DMRT3\|58524; DNAJB4\|11080; |
| | DNM3\|26052; DNMT3L\|29947; DNTT\|1791; DPH3B\|100132911; DSC1\|1823; DSEL\|92126; |
| | DSTYK\|25778; DUSP13\|51207; DUSP14\|11072; DYM\|54808; DYRK3\|8444; ECM1\|1893; |
| | EDNRA\|1909; EFCAB1\|79645; EHBP1\|23301; E1F2AK4\|440275; E1F3A\|8661; EIF4A3\|9775; |
| | EIF4E1B\|253314; ELOVL4\|6785; EMP1\|2012; EMP3\|2014; ENDOD1\|23052; ENKUR\|219670; |
| | ENPP1\|5167; ENTPD2\|954; EPDR1\|54749; EPHB3\|2049; EPHB4\|2050; EPN2\|22905; EPRS\|2058; |
| | ERC1\|23085; ERMN\|57471; ESD\|2098; ESF1\|51575; ESYT2\|57488; ETF1\|2107; EVC2\|132884; |
| | EXTL3\|2137; EYS\|346007; F10\|2159; F13A1\|2162; F2RL2\|2151; FAM101B\|359845; FAM110B\|90362; |
| | FAM126A\|84668; FAM129B\|64855; FAM168A\|23201; FAM180A\|389558; FAM20C\|56975; |
| | FAM25A\|643161; FAM25B\|100132929; FAM27B\|100133121; FAM43A\|131583; FAM49A\|81553; |
| | FAM5C\|339479; FASN\|2194; FGF1\|2246; FGF12\|2257; FGF19\|9965; FHL3\|2275; FJX1\|24147; |
| | FKBP10\|60681; FKBP14\|55033; FKBP9\|11328; FLJ42709\|441094; FLJ43390\|646113; FLRT2\|23768; |
| | FN1\|2335; FNTB\|2342; FOLR2\|2350; FOXI1\|2299; FOXL1\|2300; FRG2\|448831; FRG2B\|441581; |
| | FUT11\|170384; G6PD\|2539; GABRA3\|2556; GABRG1\|2565; GALK1\|2584; GANAB\|23193; |
| | GAS7\|8522; GBX2\|2637; GCG\|2641; GEMIN5\|25929; GFPT2\|9945; GGTLC1\|92086; GJA1\|2697; |
| | GLCE\|26035; GLI2\|2736; GLT25D1\|79709; GNA12\|2768; GOLGA8G\|283768; GPC1\|2817; |
| | GPHN\|10243; GPR32\|2854; GPR37\|2861; GPSM2\|29899; GPX8\|493869; GRAMD2\|196996; |
| | GRB14\|2888; GRIK2\|2898; GRK5\|2869; GTF2A1\|2957; GUCA1A\|2978; GUCY1B3\|2983; |
| | GULP1\|51454; GXYLT2\|727936; HAUS2\|55142; HDAC4\|9759; HDAC5\|10014; HDLBP\|3069; |
| | HECW1\|23072; HEPACAM2\|253012; HEYL\|26508; HHIPL2\|79802; HIPK2\|28996; HOXC5\|3222; |
| | HOXC8\|3224; HPD\|3242; HSPA6\|3310; HTRA4\|203100; IARS2\|55699; ICAM5\|7087; IFT122\|55764; |
| | IGF1\|3479; IGF2BP3\|10643; IGF2R\|3482; IGFL2\|147920; IL12A\|3592; IL31RA\|133396; IMPDH1\|3614; |
| | INHBB\|3625; INS\|3630; INSRR\|3645; IPO11\|51194; IPO4\|79711; IQGAP1\|8826; ITFG1\|81533; |
| | ITGA1\|3672; ITGB8\|3696; JAG1\|182; JDP2\|122953; KANK4\|163782; KCNE4\|23704; KCNH2\|3757; |
| | KCNU1\|157855; KCTD20\|222658; KCTD4\|386618; KDELC2\|143888; KDSR\|2531; KIAA0087\|9808; |
| | KIAA0090\|23065; KIAA0391\|9692; KIAA1328\|57536; KIAA1598\|57698; KIAA1919\|91749; |
| | KIF1B\|23095; KIF25\|3834; KIF26A\|26153; KIFAP3\|22920; KLHDC10\|23008; KLRG2\|346689; |
| | KPNB1\|3837; KRT23\|25984; KRT4\|3851; KRT79\|338785; KRTAP5-2\|440021; KRTDAP\|388533; |
| | L1TD1\|54596; LAMC1\|3915; LCN1\|3933; LDLR\|3949; LDLRAD3\|143458; LEPROT\|54741; |
| | LGALS1\|3956; LGTN\|1939; LHFP\|10186; LIN28A\|79727; LIN28B\|389421; LMAN1\|3998; |
| | LOC100192378\|100192378; LOC100216001\|100216001; LOC151162\|151162; LOC338588\|338588; |
| | LOC441208\|441208; LOX\|4015; LRP1\|4035; LRP12\|29967; LRP1B\|53353; LTBP1\|4052; LYVE1\|10894; |
| | MAFG\|4097; MAGEB16\|139604; MAN2A1\|4124; MAP1A\|4130; MAP1B\|4131; MAP2\|4133; |
| | MAP2K1\|5604; MAP7D1\|55700; MAP7D3\|79649; MAPK3\|5595; MARVELD1\|83742; |
| | MBOAT2\|129642; MDGA2\|161357; ME1\|4199; MED19\|219541; MEP1B\|4225; MESTIT1\|317751; |
| | MFF\|56947; MFSD11\|79157; MGC12916\|84815; MGC4473\|79100; MGC45800\|90768; MMP16\|4325; |
| | MMS19\|64210; MPRIP\|23164; MRO\|83876; MRPL37\|51253; MT1A\|4489; MTMR2\|8898; |
| | MXRA7\|439921; MYADM\|91663; MYH10\|4628; MYO5A\|4644; MYO9A\|4649; NAMPT\|10135; |
| | NAV3\|89795; NBAS\|51594; NCAM1\|4684; NCAPD2\|9918; NEBL\|10529; NEFL\|4747; NELF\|26012; |
| | NELL2\|4753; NES\|10763; NEURL\|9148; NGF\|4803; NHEDC2\|133308; NID2\|22795; NKX6-2\|84504; |
| | NLN\|57486; NLRP12\|91662; NOTCH3\|4854; NPAS3\|64067; NPC1\|4864; NPHP4\|261734; NPR3\|4883; |
| | NR0B1\|190; NRCAM\|4897; NRSN2\|80023; NT5C3L\|115024; NTRK1\|4914; NTRK2\|4915; NTS\|4922; |
| | NUCKS1\|64710; NUDT11\|55190; NUP188\|23511; NXPH3\|11248; NXPH4\|11247; OBP2A\|29991; |
| | ODZ3\|55714; OLFML2B\|25903; OR2W3\|343171; OSBPL10\|114884; OSCP1\|127700; OTX1\|5013; |
| | OTX2\|5015; P4HB\|5034; PADI4\|23569; PAFAH1B2\|5049; PAM\|5066; PAPPA\|5069; PCDH12\|51294; |
| | PCDHB10\|56126; PCDHB11\|56125; PCDHB12\|56124; PCDHB7\|56129; PCDHB8\|56128; |
| | PCDHGA1\|56114; PCDHGA2\|56113; PCDHGA3\|56112; PCDHGB1\|56104; PCDHGC3\|5098; |
| | PCLO\|27445; PCOLCE2\|26577; PCSK5\|5125; PDE5A\|8654; PDE6H\|5149; PDGFC\|56034; |
| | PDGFD\|80310; PDGFRA\|5156; PDGFRB\|5159; PDIA6\|10130; PDLIM2\|64236; PEG10\|23089; |
| | PFKM\|5213; PGA3\|643834; PGA5\|5222; PGF\|5228; PGLYRP3\|114771; PGLYRP4\|57115; PGM3\|5238; |
| | PHOSPHO1\|162466; PIGS\|94005; PIK3C3\|5289; PINX1\|54984; PIP\|5304; PITX3\|5309; |
| | PIWIL3\|440822; PLA2G1B\|5319; PLAGL1\|5325; PLCZ1\|89869; PLD5\|200150; PLEKHG4B\|153478; |
| | POLR3D\|661; PPEF1\|5475; PPP2R2C\|5522; PPP2R3A\|5523; PPT2\|9374; PPY\|5539; PRDM13\|59336; |
| | PRKAR2A\|5576; PRL\|5617; PRMT5\|10419; PRND\|23627; PRNP\|5621; PROKR2\|128674; |
| | PRPF19\|27339; PRR11\|55771; PRRT4\|401399; PRSS23\|11098; PRSS27\|83886; PRSS37\|136242; |
| | PRSS8\|5652; PTF1A\|256297; PTPLB\|201562; PTPN14\|5784; PTPN21\|11099; PTPRG\|5793; PVT1\|5820; |
| | RAB5C\|5878; RAC3\|5881; RAPGEF5\|9771; RASA1\|5921; RASAL2\|9462; RASD1\|51655; |
| | RASGEF1C\|255426; RASGRP4\|115727; RBBP5\|5929; RBMS3\|27303; RBP7\|116362; RCAN1\|1827; |
| | RDX\|5962; REEP6\|92840; REG1A\|5967; RGS17\|26575; RHOXF2B\|727940; RIMBP2\|23504; |
| | RNF217\|154214; RNF26\|79102; RNF40\|9810; RPAP1\|26015; RPTOR\|57521; RTTN\|25914; |
| | RUNX2\|860; SAMD8\|142891; SARS\|6301; SC65\|10609; SCD\|6319; SCEL\|8796; SCGB2A2\|4250; |
| | SCRN1\|9805; SEC23A\|10484; SEPT7\|989; SERINC1\|57515; SERPINB10\|5273; SERPINB12\|89777; |
| | SERPINF1\|5176; SERPINI1\|5274; SFRP5\|6425; SGCB\|6443; SGTB\|54557; SH2D6\|284948; |
| | SHC4\|399694; SIDT2\|51092; SIGLEC6\|946; SLC12A2\|6558; SLC12A3\|6559; SLC13A5\|284111; |
| | SLC16A6\|9120; SLC16A9\|220963; SLC1A5\|6510; SLC1A6\|6511; SLC22A11\|55867; SLC27A6\|28965; |
| | SLC2A12\|154091; SLC2A14\|144195; SLC2A3\|6515; SLC38A11\|151258; SLC45A1\|50651; |
| | SLC47A1\|55244; SLC6A2\|6530; SLC9A3R1\|9368; SLCO3A1\|28232; SNAI2\|6591; SNX17\|9784; |
| | SNX2\|6643; SNX24\|28966; SORBS3\|10174; SORT1\|6272; SOST\|50964; SOSTDC1\|25928; |
| | SPANXC\|64663; SPNS1\|83985; SPOCK1\|6695; SPRR3\|6707; SPSB4\|92369; SPTBN2\|6712; |
| | SRP54\|6729; SRP68\|6730; SRPX\|8406; SSRP1\|6749; STAC2\|342667; STK32B\|55351; STRAP\|11171; |
| | STRN\|6801; STT3A\|3703; STX5\|6811; STXBP1\|6812; STXBP5L\|9515; STYX\|6815; SULF2\|55959; |
| | SUMF2\|25870; SUN3\|256979; SUPT16H\|11198; SUPT6H\|6830; SUSD2\|56241; SVEP1\|79987; |
| | SYDE1\|85360; TAF13\|6884; TAF4B\|6875; TAS1R3\|83756; TBC1D16\|125058; TBCD\|6904; |
| | TBXA2R\|6915; TBXAS1\|6916; TCF4\|6925; TCL1B\|9623; TEAD4\|7004; TECR\|9524; TET1\|80312; |
| | TEX2\|55852; TGFB1\|7045; TGFBR2\|7048; THBS3\|7059; THOP1\|7064; TKT\|7086; TM4SF1\|4071; |
| | TMCC2\|9911; TMEM104\|54868; TMEM109\|79073; TMEM158\|25907; TMEM17\|200728; |
| | TMEM26\|219623; TMEM48\|55706; TMEM5\|10329; TMEM61\|199964; TMPRSS15\|5651; |
| | TMTC1\|83857; TMX2\|51075; TNFAIP8L3\|388121; TNFRSF6B\|8771; TNN\|63923; TOX4\|9878; |
| | TPD52L1\|7164; TPPP3\|51673; TPST1\|8460; TRAM2\|9697; TREML3\|340206; TREML4\|285852; |
| | TRIM16\|10626; TRIM16L\|147166; TRIM9\|114088; TRIML1\|339976; TROVE2\|6738; TRPV2\|51393; |
| | TSPAN9\|10867; TSPYL6\|388951; TUBA1A\|7846; TUBAL3\|79861; TUBGCP5\|114791; TULP3\|7289; |
| | TWIST2\|117581; TXNRD1\|7296; TYRO3\|7301; UACA\|55075; UBE2QL1\|134111; UBTD1\|80019; |
| | UCHL1\|7345; UCHL5\|51377; UPK3A\|7380; USP13\|8975; USP5\|8078; UTP18\|51096; VDAC2\|7417; |
| | VIM\|7431; VKORC1\|79001; VWA5B2\|90113; WLS\|79971; WWTR1\|25937; XAGE2\|9502; |
| | XPOT\|11260; YARS\|8565; ZC3HAV1L\|92092; ZNF385D\|79750; ZNF474\|133923; ZNF532\|55205; |
| | ZNF705A\|440077; ZNF804B\|219578; ZSCAN5B\|342933; ZW10\|9183 |

### Example 3

### Correlation Between Course of Bladder Cancer and Dynamic Change of Key Gene Expression

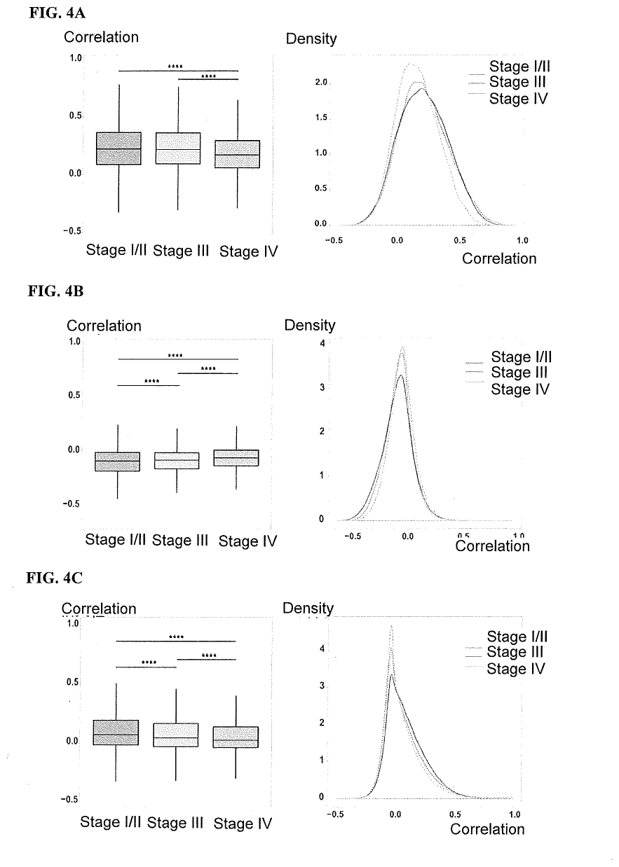

In Example 2, 1078 key genes were divided into two groups, namely, the protective effective genes and the risk effective genes. To investigate the correlation of the gene expressions inside or between the two genomes in various tumor stages of bladder cancer, the correlation coefficients of expression level of protective effective gene-protective effective gene, protective effective gene-risk effective gene and risk effective gene-risk effective gene were compared. The comparison results indicated that the correlations between genes having the same properties (i.e., protective effective gene-protective effective gene or risk effective gene-risk effective gene) or genes having different properties (i.e., protective effective gene-risk effective gene) would be significantly reduced with increased stages of bladder cancer or increased severity of condition (i.e., in accordance with the order of Stage I/II, Stage III, and Stage IV) (see, FIG. 4A-4C). For Stage I/II, Stage III, and Stage IV, FIG. 4A-4C showed the correlation coefficients of protective effective gene-protective effective gene, protective effective gene-risk effective gene or risk effective gene-risk effective gene (all abnormal values are not shown) and the corresponding density curve. Of those, *: the p value <0.05; **: the p value <0.01; ***: the p value <0.001; ****: the p value <0.0001, as detected by a double-sided Wilcoxon rank sum test.

This change could also be reflected by the variation of the corresponding density curve, that was, with increased tumor stages of bladder cancer and increased severity of condition, the density curve became higher and higher, and narrower and narrower. It can be seen that the analysis of the dynamic change in the pattern of gene expression correlations indicates that the change of the expression level of the identified key genes is closely correlated with the tumor stage (i.e., progression) of bladder cancer.

### Example4

### Construction of Co-Expression Network of Key Genes and Detection of Functional Gene Module Correlated With Clinical Feature

Test Method:

A Weighted Correlation Network Analysis (WGCNA) algorithm (see Langfelder P et al, BMC Bioinformatics 2008, 9:559) was used to construct their gene co-expression network. As compared with hard threshold filters, the WGCNA algorithm preserves all information about the target gene and its relationships through soft threshold methods. In order to obtain the correlation evidence between genes, “signed” type of the adjacency matrixes from the correlations of 1078 key genes obtained in Example 2 were selected. A gene co-expression network of the 1078 key genes in all BLCA samples was constructed by selecting an appropriate soft threshold, β=8, by use of the “pick Soft Threshold” function in the program.

In the WGCNA algorithm, a gene module is defined as a gene group comprising a number of highly linked genes in a constructed gene co-expression network. The topology overlap matrix (TOM) is obtained from the adjacency matrix by the “TOM similarity” function in the program. Based on the corresponding dissimilarity scores obtained from this topological overlap matrix, a tree view of the gene is obtained by use of the “hclust” function, and then a module identification is performed by use of the “cutreeDynamic” function. The minimum module size is set to 20. The “Mark Heat Map” function is used to generate a heat map of module-feature correlations.

Test Results:

The gene co-expression networks can provide an overall circumstance of gene-gene correlation. Based on the expression of genes in various stages of the BLCA patients, the gene co-expression networks specific to the tumor stages were constructed by use of WGCNA algorithm.

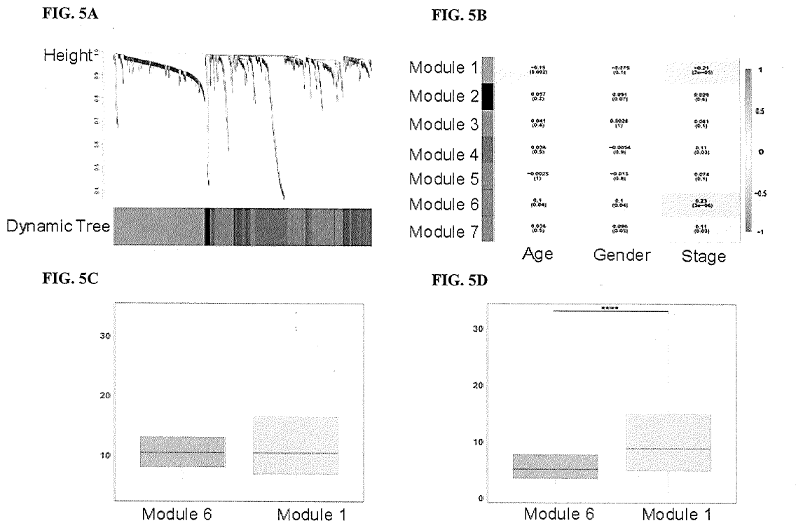

In the gene co-expression networks, the genes in the module often have similar behavior patterns. Such network modules are generally considered to have basic network topologic features, and able to provide advantageous hints of understanding the biological functions of the correlative genes in the module. To detect the functional gene module from the previously constructed gene co-expression networks, the adjacent matrix was first converted to topological overlap matrix, and provided a topological similarity score useful for the downstream module detection. Then, a dynamic tree cutting algorithm was run on a hierarchical clustering tree (i.e., a tree generated by dynamic tree cutting) generated by the WGCNA algorithm to produce seven differently sized network modules (see FIG. 5A and Table 6). FIG. 5A shows a hierarchical clustering tree (i.e., a tree diagram) constructed by WGCNA, which is derived from the dissimilarity scores represented by the various gene clusters and topological overlapping matrices derived by the dynamic tree cutting algorithm. At the bottom of FIG. 5A, various gene clusters are named in different colors; and at the left side of FIG. 5B, different numbers correspond to gene clusters represented by different colors, respectively, that is, Modules 1-7 represent the individual functional gene modules having cyan, black, yellow, brown, red, blue, and green colors, respectively.

To identify the gene modules associated with the clinical features of the BLCA patients, a correlation coefficient between the modular single genome (defined as the first major component of the gene expression profile of the corresponding module) and the clinical features of the patient with cancer was calculated (see FIG. 5B). FIG. 5B shows the relationship between the modular cells (rows) defined by the first major component of the gene expression profile in a single module and the clinical features (columns) of all the BLCA patients. Each box shows the correlation coefficient and the corresponding p value (in parentheses).

Due to the close correlation between the tumor stages and the patient survival, the gene modules associated with tumor analysis were specifically studied. It could be observed that the two gene modules had a negative correlation and a positive correlation with the bladder cancer stages, respectively (labeled by cyan and blue in FIGS. 5A-5B, respectively). In addition, it was found that most (about 93%) of the genes in the cyan module (i.e., negatively associated with the stage of bladder cancer) belong to the protective effective genes, while all the genes in the blue module (i.e., positively correlated with the stage of bladder cancer).) are risk effective genes.

The overall correlation in the blue and cyan modules (i.e., the average of the nodes in the entire network) and the correlation inside the module (i.e., the average degree of nodes within the module) were further calculated (see Table 4-Table 5, wherein Table 4 reflects the correlation of the cyan module; and Table 5 reflects the correlation of the blue module). It was found that the blue and the cyan modules showed significant differences in terms of correlation inside the modules, but there was no significant difference in their overall correlations, that is, the genes in the cyan module was more closely correlated with each other than those in the blue module (see FIG. 5C-5D). FIG. 5C shows the overall of the blue and the cyan modules, and FIG. 5D shows the correlation inside the two modules. **** indicates p-value <0.0001, as detected by double-sided Wilcoxon rank sum test.

On the basis, the genes with correlations of the first 30 modules were studied afterwards, and many of them (especially those in the blue module) have been reported in the literature to be associated with bladder cancer. For example, PDGFRB has been shown to be closely associated with recurrence of non-muscle invasive bladder cancer (see Feng J et al, PLoS One 2014, 9(5): e96671). The expression level of MARVELD1 was found to be down-regulated in several cancers including bladder cancer (see Wang S et al, Cancer Lett 2009, 282(1): 77-86). KCNE4, an ion channel gene, has been found to display abnormal expression levels in bladder cancer samples (see Biasiotta A et al J Transl Med 2016, 14(1): 285). The expression of CPT1B has been shown to be down-regulated in bladder cancer tissues, along with other genes in the carnitine-acylcarnitine metabolic pathway (see Kim W T et al, Yonsei Med J 2016, 57(4): 865-871). In addition, CKD6 has been shown to be involved in several regulatory pathways in bladder cancer (see Lu S et al, Exp Ther Med 2017, 13(6): 3309-3314). It can be seen that genes with high connectivity in the network module may also have important biological functions in the bladder cancer stages. Thus, the above results indicate that the phase-specific correlation between the survival rate of the BLCA patients and their tumor stage can be reflected by the expression levels of different groups of key genes.

Tables 4-5: Overall Correlation and Intramodular Correlation in Cyan mid Blue Modules)

TABLE 4

| | Overall | Correlation |
|---|---|---|
| | Correlation in | inside |
| Cyan Module | Cyan Module | Cyan Module |
| ABCB9\|3457 | 5.477834 | 1.744053 |
| ACOT13\|55856 | 6.381044 | 4.123986 |
| AGAP4\|119016 | 31.81634 | 30.51181 |
| AGAP6\|414189 | 29.36915 | 27.98925 |
| AGER\|177 | 19.9853 | 18.47127 |
| AGXT2L2\|85007 | 14.22039 | 11.84592 |
| AHSA2\|130872 | 21.02909 | 19.66428 |
| AK3\|50808 | 4.99064 | 2.771529 |
| AKR1B1\|231 | 5.822114 | 1.483181 |
| AKR1B15\|441282 | 5.911118 | 1.641508 |
| ALS2CL\|259173 | 14.27227 | 12.98624 |
| ANAPC4\|29945 | 15.39186 | 14.08156 |
| ANGEL2\|90806 | 10.75342 | 8.508384 |
| ANKRD10\|55608 | 20.10984 | 18.47098 |
| ANKRD22\|118932 | 5.102295 | 3.462735 |
| ANO9\|338440 | 12.94002 | 11.47108 |
| APLF\|200558 | 5.965242 | 3.776466 |
| APOBEC3D\|140564 | 6.028405 | 4.18395 |
| APOBEC3F\|200316 | 7.066094 | 5.540659 |
| APOBEC3G\|60489 | 4.832917 | 3.12444 |
| APOL1\|8542 | 6.182556 | 4.769884 |
| APOL2\|3780 | 6.928427 | 5.344402 |
| APOL4\|80832 | 6.158234 | 4.890135 |
| APOL6\|80830 | 4.751629 | 1.800271 |
| ARRDC2\|27106 | 11.85753 | 9.786373 |
| ASB13\|79754 | 5.589287 | 4.093198 |
| ATF7IP2\|80063 | 10.33559 | 9.135336 |
| ATOH8\|84913 | 8.905037 | 7.43206 |
| BAT1\|7919 | 12.03116 | 9.419455 |
| BATF\|10538 | 9.38761 | 8.014908 |
| BCL2L14\|79370 | 5.032201 | 3.707667 |
| BLOC1S3\|388552 | 6.406508 | 4.872376 |
| BTN2A1\|11120 | 8.122045 | 5.29039 |
| C11orf66\|220004 | 10.44755 | 8.699841 |
| C13orf39\|196541 | 10.19418 | 7.37013 |
| C15orf58\|390637 | 8.296109 | 6.994275 |
| C17orf86\|654434 | 14.67192 | 12.53005 |
| C19orf6\|91304 | 17.91574 | 16.37403 |
| C19orf66\|55337 | 8.90338 | 6.935171 |
| C19orf71\|100128569 | 10.67142 | 8.993376 |
| C1orf126\|200197 | 19.60872 | 18.65484 |
| C1orf159\|54991 | 23.32437 | 22.02795 |
| C1orf213\|148898 | 22.43741 | 21.19846 |
| C1orf63\|57035 | 19.4695 | 18.25647 |
| C20orf196\|149840 | 4.980749 | 2.769479 |
| C20orf96\|140680 | 14.03458 | 12.2192 |
| C22orf43\|51233 | 9.520324 | 7.810369 |
| C2orf60\|129450 | 7.426214 | 5.232193 |
| C2orf63\|130162 | 19.02092 | 17.83396 |
| C3orf19\|51244 | 12.83723 | 11.5565 |
| C3orf23\|285343 | 9.872907 | 8.834486 |
| C3orf62\|375341 | 16.54428 | 15.39259 |
| C4orf21\|55345 | 11.86983 | 9.743229 |
| C5orf56\|441108 | 7.364758 | 3.970657 |
| C6orf115\|58527 | 5.931571 | 4.717021 |
| C6orf134\|79969 | 13.24103 | 11.65404 |
| C6orf136\|221545 | 12.8006 | 11.41923 |
| C6orf47\|57827 | 5.622218 | 3.862053 |
| C6orf62\|81688 | 6.89753 | 4.748752 |
| C8orf44\|56260 | 12.81691 | 11.02788 |
| CALML3\|810 | 5.716297 | 1.197602 |
| CARD11\|84433 | 8.332518 | 7.348336 |
| CARD9\|64170 | 4.745875 | 1.905874 |
| CASP9\|842 | 10.56899 | 9.376432 |
| CCDC130\|81576 | 29.81135 | 28.36282 |
| CCDC14\|64770 | 22.47387 | 20.83231 |
| CCDC24\|149473 | 15.5272 | 14.09882 |
| CCNJL\|79616 | 4.465669 | 2.806769 |
| CCNL1\|57018 | 20.01031 | 18.69198 |
| CCNL2\|81669 | 34.28785 | 33.12275 |
| CCNT2\|905 | 14.7962 | 13.54894 |
| CCRL2\|9034 | 4.751551 | 2.902378 |
| CCT6P1\|643253 | 14.59447 | 13.05454 |
| CD96\|10225 | 6.209782 | 4.881115 |
| CDC42EP5\|148170 | 8.658783 | 7.264479 |
| CDK10\|8558 | 22.81239 | 21.33481 |
| CDK3\|1018 | 27.48636 | 26.1045 |
| CEACAM16\|388551 | 8.040437 | 5.432962 |
| CELF6\|60677 | 9.855245 | 7.695039 |
| CHKB-CPT1B\|386593 | 31.36763 | 30.23801 |
| CHMP4C\|92421 | 5.375063 | 3.940475 |
| CIRI\|9541 | 7.406743 | 6.037605 |
| CLDNI5\|24146 | 8.093615 | 5.92883 |
| CLEC2D\|29121 | 7.689356 | 5.424053 |
| CLK1\|1195 | 19.19412 | 17.78341 |
| CNKSR1\|10256 | 18.30573 | 17.36181 |
| COLQ\|8292 | 16.12491 | 14.65172 |
| COX7B\|1349 | 8.045075 | 6.154211 |
| COX8C\|341947 | 9.249511 | 6.539525 |
| CPT1B\|1375 | 23.3933 | 22.03905 |
| CRB3\|92359 | 11.91416 | 10.80861 |
| CROCCL1\|84809 | 16.83292 | 15.06859 |
| CRTC2\|200186 | 8.798996 | 7.031908 |
| CSAD\|51380 | 25.20462 | 23.87074 |
| CTRL\|1506 | 6.633586 | 4.400006 |
| CTU1\|90353 | 9.608321 | 7.177278 |
| CYorf15B\|84663 | 10.61341 | 9.078494 |
| CYP2C8\|1558 | 15.04377 | 13.72835 |
| CYP4Z1\|199974 | 6.830522 | 5.043863 |
| CYTH2\|9266 | 16.73179 | 15.27385 |
| DCAF4L1\|285429 | 8.728938 | 6.846966 |
| DCDC2B\|149069 | 5.990142 | 4.198565 |
| DEDD2\|162989 | 6.291006 | 4.777805 |
| DEGS2\|123099 | 8.512336 | 7.321962 |
| DMTF1\|9988 | 21.58423 | 19.62375 |
| DNAH1\|25981 | 13.04594 | 11.17844 |
| DNASE1L2\|1775 | 13.92555 | 12.08852 |
| DOK\|7285489 | 9.772867 | 8.502783 |
| DOM3Z\|1797 | 12.67981 | 10.56134 |
| ECHDC2\|55268 | 26.79565 | 25.77886 |
| EFHD2\|79180 | 6.31192 | 5.123317 |
| ELF4\|2000 | 5.409014 | 4.004626 |
| ELMOD3\|84173 | 17.84128 | 16.4241 |
| ENGASE\|64772 | 22.43419 | 21.23714 |
| ERCC5\|2073 | 12.1715 | 11.03147 |
| ETV7\|51513 | 6.257621 | 3.980334 |
| FAAH\|2166 | 18.12 | 17.09626 |
| FAAH2\|158584 | 6.830963 | 5.180738 |
| FAM113B\|91523 | 6.133204 | 4.606014 |
| FAM122B\|159090 | 8.310399 | 6.771675 |
| EAM13A\|10144 | 9.595964 | 8.245528 |
| FAM166B\|730112 | 4.86653 | 2.801875 |
| FAM193B\|54540 | 29.64834 | 28.32007 |
| FAM200B\|285550 | 10.59829 | 9.159282 |
| FAM25A\|643161 | 10.49674 | 7.752352 |
| FAM25B\|100132929 | 9.580697 | 7.003081 |
| FAM73B\|84895 | 17.12398 | 15.86081 |
| FANCF\|2188 | 6.897586 | 4.768119 |
| FBP1\|2203 | 10.69072 | 9.723552 |
| FBXO46\|23403 | 10.90846 | 9.474353 |
| FBXO6\|26270 | 4.716961 | 2.573429 |
| FCHSD1\|89848 | 22.86469 | 21.74014 |
| FER1L4\|80307 | 26.9798 | 26.06831 |
| FITM1\|161247 | 12.55968 | 10.70741 |
| FIJI\|2825\|440101 | 12.99091 | 11.25689 |
| FNBP4\|23360 | 19.35449 | 17.82523 |
| FOXD4L1\|200350 | 13.66608 | 12.20072 |
| GAK\|2580 | 11.23625 | 9.952901 |
| GBA2\|57704 | 10.08365 | 7.558709 |
| GEMIN8\|54960 | 17.89458 | 16.76461 |
| GGA1\|26088 | 19.51411 | 17.94414 |
| GGTLC1\|92086 | 10.23632 | 7.155106 |
| GK\|2710 | 5.373035 | 3.49107 |
| GLTSCR1\|29998 | 17.54171 | 16.32455 |
| GLYCTK\|132158 | 8.534931 | 6.690525 |
| GMIP\|51291 | 10.30856 | 9.140789 |
| GOLGA2B\|55592 | 17.62546 | 16.03707 |
| GRIPAP1\|56850 | 7.056478 | 5.422625 |
| GSDMB\|55876 | 13.50669 | 12.52347 |
| HCG26\|352961 | 6.381155 | 3.571162 |
| HCG27\|253018 | 6.952419 | 4.607102 |
| HCG4P6\|80868 | 6.491099 | 4.410092 |
| HDAC10\|83933 | 18.15317 | 16.86909 |
| HDHD3\|81932 | 9.401756 | 7.97115 |
| HEXDC\|284004 | 15.53542 | 13.97894 |
| HIPIR\|9026 | 15.24876 | 14.15623 |
| HIST1H2BL\|8340 | 5.445219 | 2.788151 |
| HIST1H4C\|8364 | 5.872322 | 3.191386 |
| HIST1H4J\|8363 | 7.887398 | 5.891447 |
| HIST2H2AC\|8338 | 9.622713 | 7.665179 |
| HLA-L\|3139 | 5.922755 | 3.105731 |
| HNMT\|3176 | 7.457185 | 6.059481 |
| HOXB5\|3215 | 10.62304 | 9.154597 |
| HOXB7\|3217 | 9.267802 | 7.9704 |
| HSH2D\|84941 | 11.04672 | 9.781971 |
| HSPA1L\|3305 | 6.557008 | 4.123867 |
| ID2B\|84099 | 7.747969 | 6.071335 |
| IDUA\|3425 | 19.26332 | 17.9001 |
| IFT27\|11020 | 14.57003 | 13.22262 |
| IKBKB\|3551 | 11.61332 | 10.47827 |
| INSL3\|3640 | 6.972142 | 4.373775 |
| IP6K2\|51447 | 23.31224 | 22.09011 |
| IRFI\|3659 | 7.032037 | 3.433866 |
| KCNJ15\|3772 | 6.124477 | 4.912261 |
| KIAA0907\|22889 | 21.0294 | 19.47434 |
| KIAA1530\|57654 | 17.71297 | 16.38034 |
| KIAA1875\|340390 | 11.6349 | 10.01482 |
| KIF21B\|23046 | 6.64563 | 3.984605 |
| KIF25\|3834 | 9.897006 | 6.930618 |
| KLHL36\|79786 | 6.184693 | 4.597239 |
| KLRA1\|10748 | 17.96575 | 16.64705 |
| KRT23\|25984 | 5.259303 | 1.429667 |
| LENG8\|114823 | 16.82126 | 15.19134 |
| LIME1\|54923 | 10.20363 | 8.139929 |
| LIPT1\|51601 | 8.455576 | 7.074986 |
| LMBR1L\|55716 | 19.62435 | 18.5135 |
| LOC100129637\|100129637 | 16.90884 | 15.25837 |
| LOC100144604\|100144604 | 9.444176 | 7.986742 |
| LOC100272146\|100272146 | 7.934453 | 6.141907 |
| LOC100286793\|100286793 | 5.820365 | 3.241588 |
| LOC100288778\|100288778 | 15.35939 | 13.9554 |
| LOC146880\|146880 | 21.84899 | 20.64445 |
| LOC221442\|221442 | 13.60515 | 12.26965 |
| LOC283314\|283314 | 7.016398 | 4.25478 |
| LOC284232\|284232 | 16.26431 | 14.78657 |
| LOC284233\|284233 | 15.86769 | 14.43893 |
| LOC284900\|284900 | 19.38433 | 17.80758 |
| LOC285074\|285074 | 10.71887 | 8.726309 |
| LOC285359\|285359 | 12.98196 | 11.5447 |
| LOC388692\|388692 | 10.60983 | 9.119242 |
| LOC391322\|391322 | 13.46223 | 11.89199 |
| LOC400927\|400927 | 10.15379 | 8.25913 |
| LOC401052\|401052 | 9.375472 | 7.494808 |
| LOC440944\|440944 | 10.4417 | 8.760527 |
| LOC642846\|642846 | 19.70623 | 18.12494 |
| LOC91316\|91316 | 20.89732 | 19.50587 |
| LUC7L\|55692 | 30.38672 | 28.83583 |
| LY6G5B\|58496 | 11.93231 | 9.8727 |
| MAGEB16\|139604 | 5.596637 | 1.718861 |
| MAPK8IP3\|23162 | 27.72589 | 26.49489 |
| ME3\|10873 | 15.67693 | 14.30617 |
| MFAP3L\|9848 | 8.961033 | 7.857667 |
| MFSD2A\|84879 | 4.40654 | 2.938799 |
| MRFAP1L1\|114932 | 5.608314 | 3.984897 |
| MRPS6\|64968 | 4.359446 | 1.777187 |
| MSL3\|10943 | 5.806092 | 3.652484 |
| MST1R\|4486 | 6.864897 | 5.416122 |
| MTERFD3\|80298 | 17.66065 | 15.94403 |
| MZF1\|7593 | 26.33303 | 25.10378 |
| NADSYN1\|55191 | 18.43657 | 17.50168 |
| NBR2\|10230 | 9.998711 | 8.588838 |
| NCRNA00105\|80161 | 20.3412 | 18.68782 |
| NCRNA00115\|79854 | 14.2597 | 12.60527 |
| NDOR1\|27158 | 8.868186 | 6.96213 |
| NFKBID\|84807 | 11.23387 | 9.546071 |
| NFYA\|4800 | 7.215941 | 5.452685 |
| NPAS2\|4862 | 11.45407 | 10.56953 |
| NPIPL3\|23117 | 26.3113 | 24.89084 |
| NR2F6\|2063 | 16.44082 | 15.19925 |
| NSUN5P1\|155400 | 23.52386 | 21.7444 |
| NSUN5P2\|269294 | 24.74286 | 23.04363 |
| NSUN6\|221078 | 19.77804 | 18.71947 |
| NUDT16P1\|152195 | 5.788355 | 4.402399 |
| NUDT19\|390916 | 5.127266 | 3.136472 |
| OAS1\|4938 | 4.887764 | 3.496281 |
| OFD1\|8481 | 21.04276 | 19.85606 |
| ORMDL1\|94101 | 13.16641 | 11.63414 |
| ORMDL3\|94103 | 8.251339 | 7.084864 |
| P2RY11\|5032 | 12.12945 | 9.881631 |
| P4HB\|5034 | 5.965058 | 1.743061 |
| PAQR6\|79957 | 10.68752 | 8.728874 |
| PAR1\|145624 | 8.593502 | 7.048349 |
| PARP4\|143 | 5.574615 | 3.901292 |
| PATL2\|197135 | 6.215167 | 3.070691 |
| PBOV1\|59351 | 8.052115 | 6.70591 |
| PCF11\|51585 | 14.82712 | 13.45844 |
| PDCL3\|79031 | 5.078479 | 3.206331 |
| PDXDC2\|283970 | 18.16582 | 17.03011 |
| PGPEP1\|54858 | 13.46394 | 12.08172 |
| PIGA\|5277 | 4.790925 | 2.973031 |
| PION\|54103 | 7.350285 | 5.410442 |
| PIWIL3\|440822 | 4.940142 | 1.681777 |
| PLA2G6\|8398 | 21.95075 | 20.6389 |
| PLEKHA6\|22874 | 11.55484 | 10.34337 |
| PLEKHH1\|57475 | 18.43447 | 17.14679 |
| PLGLB2\|5342 | 13.31189 | 11.47448 |
| PLIN5\|440503 | 14.1615 | 12.98385 |
| PLXNB1\|5364 | 24.65183 | 23.53078 |
| PMS1\|5378 | 7.752948 | 5.482885 |
| PMS2L3\|5387 | 15.59102 | 14.13082 |
| POLB\|5423 | 4.138356 | 2.172015 |
| PPFIBP2\|8495 | 16.46626 | 15.59512 |
| PRICKLE3\|4007 | 7.029037 | 5.610721 |
| PRKD2\|25865 | 7.66691 | 6.288953 |
| PRSS27\|83886 | 6.738052 | 3.608051 |
| PSMB10\|5699 | 8.230521 | 6.076476 |
| PSMB8\|5696 | 7.337279 | 3.902714 |
| PTPN6\|5777 | 8.225909 | 6.68406 |
| PYROXD2\|84795 | 13.99822 | 12.66306 |
| RABL2A\|11159 | 9.843486 | 7.523836 |
| RAD9A\|5883 | 15.4357 | 14.03285 |
| RASGEF1C\|255426 | 6.127558 | 1.717751 |
| RBCK1\|10616 | 7.73093 | 5.53174 |
| RBM6\|10180 | 24.53842 | 23.22358 |
| REEP6\|92840 | 9.880192 | 7.959849 |
| REV1\|51455 | 7.795962 | 6.256679 |
| RG9MTD3\|158234 | 11.85013 | 10.08577 |
| RGPD6\|729540 | 10.6672 | 9.11789 |
| RGS17\|26575 | 5.581623 | 1.684367 |
| RPL32P3\|132241 | 17.14246 | 15.25095 |
| RPP21\|79897 | 12.97309 | 11.53783 |
| RTP2\|344892 | 5.358445 | 3.335444 |
| RTP4\|64108 | 5.292846 | 3.335848 |
| RWDD3\|25950 | 8.239732 | 6.734428 |
| SCGB2A2\|4250 | 4.64805 | 1.484925 |
| SCXB\|642658 | 14.70029 | 1.28163 |
| SDCBP2\|127111 | 6.219454 | 4.87193 |
| SEC31B\|25956 | 15.03909 | 12.4755 |
| SEMA4D\|10507 | 5.687389 | 3.319947 |
| SEPT7P2\|641977 | 17.61252 | 15.63689 |
| SER1NC4\|619189 | 19.27247 | 17.79336 |
| SETMAR\|6419 | 9.911912 | 8.580758 |
| SFRS16\|1129 | 24.21007 | 22.95881 |
| SFRS17A\|8227 | 14.67536 | 13.19601 |
| SH3GLB2\|56904 | 17.23626 | 16.08434 |
| SHC3\|53358 | 5.580995 | 3.844049 |
| SKINTL\|391037 | 11.65639 | 9.97521 |
| SLC10A5\|347051 | 9.579309 | 7.977419 |
| SLCIA6\|6511 | 4.813263 | 1.791047 |
| SLC25A34\|284723 | 7.274232 | 5.140837 |
| SLC45A3\|85414 | 6.834352 | 5.421354 |
| SLC5A9\|200010 | 6.129555 | 3.619823 |
| SLC6A2\|6530 | 9.897217 | 6.846616 |
| SLC7A9\|11136 | 6.500446 | 4.472883 |
| SMAD6\|4091 | 8.709323 | 7.354959 |
| SP140L\|93349 | 5.528713 | 3.333537 |
| SPDYA\|245711 | 21.74397 | 20.40279 |
| SPG7\|6687 | 17.6.3375 | 16.06227 |
| SPOCD1\|90853 | 10.88838 | 9.623722 |
| SPSB3\|90864 | 21.14191 | 19.69228 |
| STAG3L3\|442578 | 11.8995 | 9.991007 |
| STAP2\|55620 | 14.46051 | 13.38422 |
| STAT6\|6778 | 10.49886 | 9.229399 |
| SYCP3\|50511 | 9.044773 | 7.042883 |
| TAF1C\|9013 | 22.9386 | 21.50097 |
| TBC1D3\|729873 | 28.97206 | 27.68081 |
| TBC1D3B\|414059 | 28.94549 | 27.64839 |
| TBC1D3P2\|440452 | 11.77211 | 9.901267 |
| TCEANC\|170082 | 6.903733 | 4.643725 |
| TCTE3\|6991 | 18.60029 | 17.02534 |
| TECR\|9524 | 6.595856 | 4.031214 |
| THUMPD2\|80745 | 13.68897 | 12.35451 |
| TIA1\|7072 | 24.04186 | 22.82855 |
| TMC7\|79905 | 14.28062 | 13.37474 |
| TMEM51\|55092 | 11.26155 | 10.34009 |
| TNFAIP2\|7127 | 9.909038 | 8.968554 |
| TNK1\|8711 | 7.192411 | 5.851428 |
| TOP3B\|8940 | 13.63757 | 12.13604 |
| TRAPPC2\|6399 | 11.25546 | 10.00041 |
| TRIM26\|7726 | 5.508255 | 4.107757 |
| TRIM27\|5987 | 9.357543 | 7.55447 |
| TRIM38\|10475 | 6.456025 | 5.088441 |
| TRPV1\|7442 | 15.28438 | 13.65457 |
| TSPAN14\|81619 | 8.563321 | 7.467451 |
| TSPYL6\|388951 | 4.50218 | 1.778208 |
| TTLL3\|26140 | 30.13044 | 28.9584 |
| UBD\|10537 | 5.610032 | 2.811204 |
| UCP3\|7352 | 14.30788 | 12.59246 |
| UNC93B1\|81622 | 7.763666 | 6.578028 |
| UPK3A\|7380 | 7.272182 | 5.197534 |
| USF1\|7391 | 7.025492 | 5.262917 |
| WASH3P\|374666 | 25.09869 | 23.74321 |
| WASH7P\|653635 | 28.63511 | 27.43092 |
| WDR52\|55779 | 22.54088 | 21.39501 |
| WDR6\|11180 | 18.15303 | 16.67556 |
| YTHDC1\|91746 | 10.86094 | 8.826721 |
| ZCWPW1\|55063 | 7.831115 | 6.083326 |
| ZFPM1\|161882 | 14.63273 | 13.27058 |
| ZNF100\|163227 | 15.78214 | 14.38227 |
| ZNF137\|7696 | 17.8921 | 16.77434 |
| ZNF160\|90338 | 17.53884 | 16.22887 |
| ZNF165\|7718 | 7.625495 | 6.125923 |
| ZNF169\|169841 | 10.17908 | 7.745575 |
| ZNF182\|7569 | 19.56101 | 18.17 |
| ZNF187\|7741 | 10.63295 | 8.171808 |
| ZNF193\|7746 | 20.61592 | 19.18829 |
| ZNF195\|7748 | 16.81893 | 15.4151 |
| ZNF254\|9534 | 13.15267 | 11.5224 |
| ZNF443\|10224 | 23.88888 | 22.81372 |
| ZNF480\|147657 | 10.75078 | 9.587031 |
| ZNF493\|284443 | 18.54166 | 17.2403 |
| ZNF506\|440515 | 18.92096 | 17.87655 |
| ZNF513\|130557 | 21.90681 | 20.69157 |
| ZNF524\|147807 | 11.32494 | 9.496758 |
| ZNF564\|163050 | 16.05829 | 1488475 |
| ZNF577\|84765 | 15.32286 | 13.90218 |
| ZNF600\|162966 | 18.13161 | 17.11223 |
| ZNF630\|57232 | 12.79631 | 11.46344 |
| ZNF638\|27332 | 14.98361 | 13.12917 |
| ZNF705A\|440077 | 4.489735 | 1.778573 |
| ZNF708\|7562 | 14.72619 | 13.21463 |
| ZNF763\|284390 | 25.19442 | 24.04222 |
| ZNF799\|90576 | 22.00174 | 20.89021 |
| ZNF814\|730051 | 19.41652 | 18.0971 |
| ZNF823\|55552 | 17.8819 | 16.72755 |
| ZNF841\|284371 | 12.64709 | 11.11573 |
| ZNRD1\|30834 | 7.686867 | 6.102825 |
| ZRANB2\|9406 | 11.09344 | 8.671744 |
| ZRSR2\|8233 | 12.89957 | 11.35714 |
| ZSCAN16\|80345 | 12.8668 | 11.376 |
| ZSCAN5B\|342933 | 7.936521 | 5.284159 |

TABLE 5

| | Overall | Correlation |
|---|---|---|
| | Correlation in | inside |
| Blue Module | Blue Module | Blue Module |
| ABCC9\|10060 | 13.13097 | 8.35915 |
| ACVR1\|90 | 12.9737 | 8.074888 |
| ADAMTS12\|81792 | 10.97046 | 7.038643 |
| ADAMTS16\|170690 | 12.91018 | 8.826261 |
| ADAMTS18\|170692 | 7.842351 | 2.426652 |
| ADAMTSL1\|92949 | 12.30389 | 6.536282 |
| ADCY7\|113 | 14.81676 | 8.115218 |
| AHNAK\|79026 | 11.61155 | 6.509888 |
| ALAS2\|212 | 6.606809 | 2.76708 |
| ALDH1L2\|160428 | 14.74237 | 9.240544 |
| ANPEP\|290 | 8.206162 | 3.997661 |
| ANχA1\|301 | 9.934962 | 5.136543 |
| ANχA2\|302 | 13.03304 | 8.144519 |
| ANχA2P1\|303 | 12.09204 | 7.47647 |
| ANXA2P2\|304 | 13.93089 | 8.902993 |
| ANXA5\|308 | 17.82176 | 10.8724 |
| ARCN1\|372 | 8.215289 | 3.984781 |
| ARHGAP29\|9411 | 7.944162 | 3.141984 |
| ARL10\|285598 | 10.55155 | 4.337158 |
| ARL4C\|10123 | 15.02817 | 9.591315 |
| ARMC9\|80210 | 16.12858 | 9.887214 |
| ARSI\|340075 | 12.26275 | 7.369992 |
| ATF6\|22926 | 6.991618 | 2.683127 |
| ATG9A\|79065 | 8.955216 | 4.038752 |
| ATP2B4\|493 | 8.357154 | 4.22214 |
| ATP6V0D1\|9114 | 7.516853 | 3.298198 |
| ATP6V1B2\|526 | 12.02187 | 5.76094 |
| BACE1\|23621 | 14.34184 | 8.835548 |
| BAIAP2\|10458 | 7.354748 | 3.145949 |
| BARX2\|8538 | 7.592111 | 3.208997 |
| C13orf15\|28984 | 12.83524 | 8.253818 |
| C13orf33\|84935 | 13.93916 | 10.16322 |
| C14orf37\|145407 | 10.85355 | 6.141901 |
| C15orf38\|348110 | 7.538243 | 3.570002 |
| C15orf54\|400360 | 6.92396 | 3.328511 |
| C17orf51\|339263 | 11.41931 | 5.552822 |
| C19orf59\|199675 | 7.771466 | 4.402957 |
| C5orf62\|85027 | 15.46133 | 10.93331 |
| C6orf138\|442213 | 6.126775 | 2.653129 |
| C6orf72\|116254 | 8.732508 | 4.82973 |
| CALU\|813 | 19.17093 | 13.10348 |
| CAPN2\|824 | 12.1286 | 7.328294 |
| CBLN4\|140689 | 5.670758 | 2.224679 |
| CCDC8\|83987 | 10.81159 | 6.428594 |
| CCDC80\|151887 | 16.30421 | 11.68408 |
| CD109\|135228 | 14.74307 | 9.149505 |
| CD276\|80381 | 12.50832 | 7.548895 |
| CD300LG\|146894 | 12.60023 | 8.70988 |
| CDK6\|1021 | 11.71512 | 6.009829 |
| CERCAM\|51148 | 14.21425 | 8.885395 |
| CHPF2\|54480 | 10.25568 | 5.73971 |
| CHRNA1\|1134 | 8.248898 | 2.761088 |
| CHSY1\|22856 | 15.23113 | 10.1951 |
| CIDEC\|63924 | 11.69002 | 7.795972 |
| CKAP4\|10970 | 10.70137 | 5.689388 |
| CKMT2\|1160 | 5.952701 | 2.203285 |
| CLEC11A\|6320 | 12.09828 | 5.760021 |
| CNIH\|10175 | 9.723282 | 4.764119 |
| CNN3\|1266 | 10.10571 | 5.568823 |
| CNTNAP3\|79937 | 7.907616 | 3.795006 |
| COL18A1\|80781 | 17.47177 | 11.69545 |
| COL4A2\|1284 | 14.50558 | 8.918225 |
| COL5A1\|1289 | 17.3722 | 12.988 |
| COL5A3\|50509 | 15.83705 | 10.95568 |
| COL6A1\|291 | 16.87165 | 10.26674 |
| COPS8\|10920 | 14.73249 | 8.720556 |
| COPZ2\|51226 | 15.35109 | 10.38111 |
| CPXM1\|56265 | 12.32833 | 8.049582 |
| CRTAP\|10491 | 14.38447 | 8.410304 |
| CSGALNACT2\|55454 | 13.92111 | 9.336234 |
| CSFG4\|1464 | 9.832308 | 5.785988 |
| CTNNB1\|1499 | 9.552838 | 4.119779 |
| CUBN\|8029 | 8.553863 | 4.189882 |
| CXCR1\|3577 | 7.927364 | 4.597704 |
| CYTH3\|9265 | 12.23345 | 6.815371 |
| DIRAS3\|9077 | 11.14663 | 5.402666 |
| DNAJB4\|11080 | 12.70036 | 5.835276 |
| DNM3\|26052 | 6.498784 | 2.234187 |
| DSEL\|92126 | 9.199302 | 4.311026 |
| DUSP14\|11072 | 10.80434 | 5.444753 |
| DYRK3\|8444 | 13.45486 | 7.480383 |
| ECM1\|1893 | 8.647865 | 4.221466 |
| EDNRA\|1909 | 15.92454 | 11.38422 |
| EFCAB1\|79645 | 6.007121 | 2.050167 |
| EHBP1\|23301 | 13.31934 | 6.672912 |
| EIF2AK4\|440275 | 9.244758 | 4.156171 |
| EMP1\|2012 | 10.00898 | 6.062596 |
| ENDOD1\|23052 | 6.841735 | 3.455309 |
| EPHB3\|2049 | 7.791979 | 2.522652 |
| EPHB4\|2050 | 7.3662 | 3.122009 |
| ERC1\|23085 | 10.62523 | 5.296502 |
| ESYT2\|57488 | 8.449135 | 3.953741 |
| ETF1\|2107 | 12.63043 | 5.660143 |
| EXTL3\|2137 | 8.390969 | 3.461675 |
| EYS\|346007 | 5.091301 | 2.007602 |
| F10\|2159 | 13.20233 | 9.043034 |
| FAM126A\|84668 | 15.55451 | 8.512436 |
| FAM129B\|64855 | 8.352389 | 4.209874 |
| FAM168A\|23201 | 13.84029 | 5.785627 |
| FAM180A\|389558 | 15.8028 | 11.64087 |
| FAM27B\|100133121 | 6.847168 | 2.701046 |
| FAM43A\|131583 | 9.817376 | 4.380366 |
| FJX1\|24147 | 7.608031 | 3.69628 |
| FKBP10\|60681 | 15.27251 | 8.340275 |
| FKBP14\|55033 | 10.77226 | 6.564184 |
| FKBP9\|11328 | 13.14549 | 8.184031 |
| FLJ42709\|441094 | 9.574818 | 5.356179 |
| FLRT2\|23768 | 9.295983 | 5.52671 |
| FN1\|2335 | 15.00208 | 9.619922 |
| FUT11\|170384 | 9.838205 | 4.818737 |
| GABRG1\|2565 | 7.088774 | 2.5392 |
| GANAB\|23193 | 10.04282 | 3.803233 |
| GAS7\|8522 | 13.99539 | 9.352765 |
| GFPT2\|9945 | 14.86317 | 9.775954 |
| GJA1\|2697 | 6.65733 | 2.576551 |
| GLI2\|2736 | 10.83867 | 4.447219 |
| GLT25D1\|79709 | 13.78211 | 8.046792 |
| GPX8493869 | 19.67874 | 13.49456 |
| GRIK2\|2898 | 6.844378 | 2.36241 |
| GUCY1B3\|2983 | 13.13088 | 8.32358 |
| GXYLT2\|727936 | 15.30132 | 10.73147 |
| HDLBP\|3069 | 11.02774 | 6.176633 |
| HEYL\|26508 | 14.09798 | 8.146192 |
| IGF1\|3479 | 14.11602 | 9.509533 |
| IGF2R\|3482 | 14.7972 | 7.256242 |
| IGFL2\|147920 | 6.341531 | 2.501959 |
| IMPDH1\|3614 | 8.585309 | 3.537629 |
| INHBB\|3625 | 8.594306 | 3.88493 |
| IPO11\|51194 | 12.17275 | 5.0442 |
| IQGAP1\|8826 | 11.00847 | 5.818501 |
| ITFG1\|81533 | 7.665031 | 3.465635 |
| ITGA1\|3672 | 13.46296 | 8.409885 |
| ITGB8\|3696 | 8.325666 | 3.849812 |
| JAG1\|182 | 8.593126 | 4.295717 |
| JDP2\|122953 | 9.403408 | 4.582998 |
| KANK4\|163782 | 9.058062 | 4.120964 |
| KCNE4\|23704 | 16.49897 | 11.3479 |
| KCTD20\|222658 | 10.4262 | 4.785003 |
| KCTD4\|386618 | 5.782522 | 1.908818 |
| KDELC2\|143888 | 9.860506 | 4.762998 |
| KDSR\|2531 | 8.904887 | 4.28475 |
| KIAA0090\|23065 | 11.40779 | 5.072922 |
| LAMC1\|3915 | 11.90056 | 6.693291 |
| LDLR\|3949 | 8.627431 | 4.068903 |
| LDLRAD3\|143458 | 10.28119 | 4.357235 |
| LEPROT\|54741 | 11.10741 | 5.864982 |
| LHFP\|10186 | 14.81526 | 9.210522 |
| LOG100216001\|1002.16001 | 8.026656 | 4.472244 |
| LOC338588\|338588 | 6.7839 | 3.227422 |
| LOX\|4015 | 18.47556 | 13.27361 |
| LRP1\|4035 | 16.84813 | 11.21042 |
| MAP1A\|4130 | 15.67279 | 8.193052 |
| MAP2K1\|5604 | 8.142472 | 3.091089 |
| MAP7D1\|55700 | 13.00835 | 7.971284 |
| MAP7D3\|79649 | 14.00652 | 7.430164 |
| MARVELD1\|83742 | 15.21347 | 10.24967 |
| MBOAT2\|129642 | 8.019315 | 3.204674 |
| MDGA2\|161357 | 6.69674 | 2.410635 |
| MGC4473\|79100 | 7.953262 | 3.648145 |
| MMP16\|4325 | 9.850205 | 5.224658 |
| MT1A\|4489 | 9.722983 | 5.920628 |
| MTMR2\|8898 | 8.475688 | 3.52042 |
| MXRA7\|439921 | 14.52116 | 8.357898 |
| MYADM\|91663 | 15.48857 | 10.29045 |
| MYH10\|4628 | 11.42752 | 5.28876 |
| MYO5A\|4644 | 15.38078 | 8.63875 |
| MYO9A\|4649 | 10.91678 | 4.647195 |
| NAV3\|89795 | 13.38848 | 7.520991 |
| NBAS\|51594 | 10.34689 | 4.707647 |
| NGF\|4803 | 10.91194 | 5.700058 |
| NPC1\|4864 | 9.801263 | 5.098277 |
| NUDT11\|55190 | 10.53455 | 3.609057 |
| OLFML2B\|25903 | 16.95808 | 11.80794 |
| OSBPL10\|114884 | 8.358555 | 4.492866 |
| PAPPA\|5069 | 7.415503 | 3.470966 |
| PCDHB10\|56126 | 7.476116 | 2.800416 |
| PCDHB12\|56124 | 9.016309 | 3.506567 |
| PCDHB7\|56129 | 7.376505 | 2.789048 |
| PCDHGA1\|56114 | 7.770497 | 3.195238 |
| PCDHGA2\|56113 | 8.325681 | 4.244322 |
| PCDHGA3\|56112 | 7.864012 | 3.866465 |
| PCDHGC3\|5098 | 13.31706 | 8.84903 |
| PDGFC\|56034 | 16.11213 | 10.86394 |
| PDGFRA\|5156 | 12.54598 | 8.047527 |
| PDGFRB\|5159 | 20.09555 | 14.50617 |
| PDIA6\|10130 | 9.554878 | 3.653958 |
| PDLIM2\|64236 | 16.08854 | 10.20853 |
| PGM3\|5238 | 8.156451 | 3.642834 |
| PITX3\|5309 | 10.697 | 5.532875 |
| PPEF1\|5475 | 11.60144 | 6.453486 |
| PPP2R3A\|5523 | 9.451206 | 4.134681 |
| PRKAR2A\|5576 | 11.5831 | 4.488739 |
| PRNP\|5621 | 10.42986 | 5.762221 |
| PTPN14\|5784 | 9.146508 | 4.38854 |
| PTPRG\|5793 | 12.24853 | 6.283886 |
| RAB5C\|5878 | 7.200387 | 3.235563 |
| RAPGEF5\|9771 | 8.848998 | 3.565774 |
| RASAL2\|9462 | 10.01021 | 5.256871 |
| RBMS3\|27303 | 13.64648 | 6.987052 |
| RCAN1\|1827 | 7.778552 | 3.659727 |
| RDX\|5962 | 5.399014 | 1.919903 |
| RNF217\|154214 | 13.3907 | 6.004643 |
| RNF26\|79102 | 7.817711 | 2.750613 |
| RTTN\|25914 | 9.516454 | 4.509806 |
| RUNX2\|860 | 10.01364 | 5.021739 |
| SAMD8\|142891 | 9.790667 | 4.156186 |
| SC65\|10609 | 10.06049 | 4.961883 |
| SCEL\|8796 | 7.282433 | 2.915824 |
| SCRN1\|9805 | 12.08431 | 6.182751 |
| SEC23A\|10484 | 13.86916 | 8.890863 |
| SEPT7\|989 | 10.89825 | 5.647405 |
| SERINC1\|57515 | 10.31578 | 5.496427 |
| SERPINF1\|5176 | 15.61961 | 11.07712 |
| SGCB\|6443 | 16.50585 | 9.083036 |
| SGTB\|54557 | 20.26334 | 10.10089 |
| SHC4\|399694 | 7.584366 | 4.288294 |
| SIDT2\|51092 | 9.903761 | 4.769914 |
| SLC13A5\|284111 | 6.878519 | 2.805847 |
| SLC45A1\|50651 | 7.684709 | 2.97891 |
| SNAI2\|6591 | 11.94192 | 6.35909 |
| SORBS3\|10174 | 12.36973 | 6.13756 |
| SPOCK1\|6695 | 10.17512 | 6.150526 |
| SRPX\|8406 | 12.76273 | 8.134469 |
| STT3A\|3703 | 5.980666 | 2.185264 |
| STX5\|6811 | 7.84214 | 3.030981 |
| STYX\|6815 | 10.82552 | 5.162872 |
| SULF2\|55959 | 10.48009 | 6.235844 |
| SUMF2\|25870 | 6.611026 | 2.619337 |
| SVEP1\|79987 | 15.45949 | 11.27987 |
| SYDE1\|85360 | 17.61939 | 11.3254 |
| TAF13\|6884 | 11.31507 | 5.423339 |
| TBC1D16\|125058 | 9.866843 | 4.631512 |
| TEAD4\|7004 | 13.36061 | 6.546983 |
| TEX2\|55852 | 10.79517 | 4.538975 |
| TGFB1\|7045 | 12.19039 | 7.551476 |
| THBS3\|7059 | 7.937434 | 2.965675 |
| TMEM109\|79073 | 7.118344 | 3.274175 |
| TMEM158\|25907 | 14.42556 | 7.144049 |
| TMEM17\|200728 | 7.880861 | 3.220485 |
| TMTC1\|83857 | 8.275007 | 3.146538 |
| TNFAIP8L3\|388121 | 14.40217 | 9.483474 |
| TNFRSF6B\|8771 | 9.975467 | 4.938206 |
| TOX4\|9878 | 8.246825 | 2.966731 |
| TPST1\|8460 | 11.58994 | 7.531146 |
| TRAM2\|9697 | 15.58328 | 9.100544 |
| TSPAN9\|10867 | 9.594071 | 5.551382 |
| TWIST2\|117581 | 12.27405 | 7.690602 |
| UACA\|55075 | 10.91503 | 6.0295 |
| VIM\|7431 | 16.57971 | 10.4705 |
| VKORC1\|79001 | 10.46261 | 4.970255 |
| WWTR1\|25937 | 16.25919 | 10.01286 |
| ZNF474\|133923 | 7.45068 | 2.65575 |
| ZNF532\|55205 | 13.16329 | 6.190627 |

TABLE 6
7 Network Modules

| | |
|---|---|
| Black | AKR7A2\|8574; ATP13A4\|84239; C1or184\|149469; CELA3B\|23436; CTRB1\|1504; CTRB21440387; |
| Module | GCG\|2641; INS\|3630; KRTAP5-2\|440021; MAPK3\|5595; NKX6-2\|84504; PLA2G1B\|5319; PPY\|5539; |
| | PROKR2\|128674; PRSS8\|5652; PTF1A\|256297; REG1A\|5967; RNF40\|9810; SFRP5\|6425; |
| | SLC16A9\|220963; SPNS1\|83985; SUSD2\|56241 |
| Blue | ABCC9\|10060; ACVR1\|90; ADAMTS12\|81792; ADAMTS16\|170690; ADAMTS18\|170692; |
| Module | ADAMTSL1\|92949; ADCY7\|113; AHNAK\|Q79026; ALAS2\|212; ALDHIL2\|160428; ANPEP\|290; |
| | ANXA1\|301; ANXA2P1\|303; ANXA2P2\|304; ANXA2\|302; ANXA5\|308; ARCN1\|372; |
| | ARHGAP29\|9411; ARL10\|285598; ARL4C\|10123; ARMC9\|80210; ARS1\|340075; ATF6\|22926; |
| | ATG9A\|79065; ATP2B4\|493; ATP6V0D1\|9114; ATP6V1B2\|526; BACE1\|23621; BAIAP2\|10458; |
| | BARX2\|8538; C13orf15\|28984; C13orf33\|84935; C14orf37\|145407; C15orf38\|348110; C15orf54\|400360; |
| | C17orf51\|339263; C19orf59\|199675; C5orf62\|85027; C6orf138\|442213; C6orf72\|116254; CALU\|813; |
| | CAPN2\|824; C.BLN4\|140689; CCDC80\|151887; CCDC8\|83987; CD109\|135228; CD276\|80381; |
| | CD300LG\|146894; CDK6\|1021; CERCAM\|51148; CHPF2\|54480; CHRNA1\|1134; CHSY1\|22856; |
| | CIDEC\|63924; CKAP4\|10970; CKMT2\|1160; CLEC11A\|6320; CNIH\|10175; CNN3\|1266; |
| | CNTNAP3\|79937; COL18A1\|80781; COL4A2\|1284; COL5A1\|1289; COL5A3\|50509; COL6A1\|1291; |
| | COPS8\|10920; COPZ2\|51226; CPXM1\|56265; CRTAP\|10491; CSGALNACT2\|55454; CSPG4\|1464; |
| | CTNNB1\|1499; CUBN\|8029; CXCR1\|3577; CYTH3\|9265; DIRAS3\|9077; DNAJB4\|11080; DNM3\|26052; |
| | DSEL\|92126; DUSP14\|11072; DYRK3\|8444; ECM1\|1893; EDNRA\|1909; EFCAB1\|79645; |
| | EHBP1\|23301; EIF2AK4\|440275; EMP1\|2012; ENDOD1\|23052; EPHB3\|2049; EPHB4\|2050; |
| | ERC1\|23085; ESYT2\|57488; ETF1\|2107; EXTL3\|2137; EYS\|346007; F10\|2159; FAM126A\|84668; |
| | FAM129B\|64855; FAM168A\|23201; FAM180A\|389558; FAM27B\|100133121; FAM43A\|131583; |
| | FJX1\|24147; FKBP10\|60681; FKBP14\|55033; FKBP9\|11328; FLJ42709\|441094; FLRT2\|23768; |
| | FN1\|2335; FUT11\|170384; GABRG1\|2565; GANAB\|23193; GAS7\|8522; GFPT2\|9945; GJA1\|2697; |
| | GLI2\|2736; GLT25D1\|79709; GPX8\|493869; GRIK2\|2898; GUCY1B3\|2983; GXYLT2\|727936; |
| | HDLBP\|3069; HEYL\|26508; IGF1\|3479; IGF2R\|3482; IGFL2\|147920; IMPDH1\|3614; INHBB\|3625; |
| | IPO11\|51194; IQGAP1\|8826; ITFG1\|81533; ITGA1\|3672; ITGB8\|3696; JAG1\|182; JDP2\|122953; |
| | KANK4\|163782; KCNE4\|23704; KCTD20\|222658; KCTD4\|386618; KDELC2\|143888; KDSR\|2531; |
| | KIAA0090\|23065; LAMC1\|3915; LDLRAD3\|143458; LDLR\|3949; LEPROT\|54741; LHFP\|10186; |
| | LOC100216001\|100216001; LOC338588\|338588; LOX\|4015; LRP1\|4035; MAP1A\|4130; MAP2K1\|5604; |
| | MAP7D1\|55700; MAP7D3\|79649; MARVELD1\|83742; MBOAT2\|129642; MDGA2\|161357; |
| | MGC4473\|79100; MMP16\|4325; MT1A\|4489; MTMR2\|8898; MXRA7\|439921; MYADM\|91663; |
| | MYH10\|4628; MYO5A\|4644; MYO9A\|4649; NAV3\|89795; NBAS\|51594; NGF\|4803; NPC1\|4864; |
| | NUDT11\|55190; OLFML2B\|25903; OSBPL10\|114884; PAPPA\|5069; PCDHB10\|56126; |
| | PCDHB12\|56124; PCDHB7\|56129; PCDHGA1\|56114; PCDHGA2\|56113; PCDHGA3\|56112; |
| | PCDHGC3\|5098; PDGFC\|56034; PDGFRA\|5156; PDGFRB\|5159; PDIA6\|10130; PDLIM2\|64236; |
| | PGM3\|5238; PITX3\|5309; PPEF1\|5475; PPP2R3A\|5523; PRKAR2A\|5576; PRNP\|5621; PTPN14\|5784; |
| | PTPRG\|5793; RAB5C\|5878; RAPGEF5\|9771; RASAL2\|9462; RBMS3\|27303; RCAN1\|1827; RDX\|5962; |
| | RNF217\|154214; RNF26\|79102; RTTN\|25914; RUNX2\|860; SAMD8\|142891; SC65\|10609; SCEL\|8796; |
| | SCRN1\|9805; SEC23A\|10484; SEPT7\|989; SERINC1\|57515; SERPINF1\|5176; SGCB\|6443; |
| | SGTB\|54557; SHC4\|399694; SIDT2\|51092; SLC13A5\|284111; SLC45A1\|50651; SNAI2\|6591; |
| | SORBS3\|10174; SPOCK1\|6695; SRPX\|8406; STT3A\|3703; STX5\|6811; STYX\|6815; SULF2\|55959; |
| | SUMF2\|25870; SVEP1\|79987; SYDE1\|85360; TAF13\|6884; TBC1D16\|125058; TEAD4\|7004; |
| | TEX2\|55852; TGFBI\|7045; THBS3\|7059; TMEM109\|79073; TMEM158\|25907; TMEM17\|200728; |
| | TMTC1\|83857; TNFAIP8L3\|388121; TNFRSF6B\|8771; TOX4\|9878; TPST1\|8460; TRAM2\|9697; |
| | TSPAN9\|10867; TWIST2\|117581; UAC,A\|55075; VIM\|7431; VKORC1\|79001; WWTR1\|25937; |
| | ZNF474\|133923; ZNF532\|55205 |
| Brown | ABCC1\|4363; ABCE1\|6059; ACCN1\|40; ACLY\|47; ADRA2B\|151; AHCY\|191; ALPL\|249; |
| Module | AMDHD1\|144193; ANO1\|55107; AP2B1\|163; ASPM\|259266; ATP1A1\|476; ATP6V0A1\|535; |
| | ATP6V1A\|523; ATP6V1B1\|525; ATP8B2\|57198; AVIL\|10677; B3GALT2\|8707; B4GALNT2\|124872; |
| | BSND\|7809; C10orf90\|118611; C11orf16\|56673; C11orf53\|341032; C11orf87\|399947; C14orf126\|112487; |
| | C14orf128\|84837; C14orf86\|283592; C16orf63\|123811; C17orf39\|79018; C18orf20\|221241; |
| | C18orf22\|79863; C19orf26\|255057; C20orf117\|140710; C5orf13\|9315; C9orf24\|84688; CA10\|56934; |
| | CA5A\|763; CA7\|766; CACNA1B\|774; CAD\|790; CALCA\|796; CALHM3\|119395; CALM1\|801; |
| | CCDC102B\|79839; CCDC21\|64793; CCNA1\|8900; CCT6A\|908; CDC73\|79577; CEACAM21\|90273; |
| | CERK\|64781; CLDN10\|9071; CLTC\|1213; CMTM2\|146225; COBL\|23242; COG8\|84342; COPS3\|8533; |
| | CRNN49860; CSNK1A1P\|161635; CXADRP2\|646243; CXCR7\|57007; CYP19A1\|1588; DARS2\|55157; |
| | DBN1\|1627; DDX10\|1662; DMRT3\|58524; DNTT\|1791; DPH3B\|100132911; DSC1\|1823; |
| | DSTYK\|25778; EIF3A\|8661; ELOVL4\|6785; ENKUR\|219670; EPN2\|22905; EPRS\|2058; ERMN\|57471; |
| | ESD\|2098; ESF1\|51575; EVC2\|132884; FAM110B\|90362; FAM5C\|339479; FGF12\|2257; FGF1\|2246; |
| | FNTB\|2342; FOX11\|2299; FRG2B\|441581; FRG2\|448831; G6PD\|2539; GEM1N5\|25929; GPHN\|10243; |
| | GPR37\|2861; GPSM2\|29899; GRB14\|2888; GRK5\|2869; GUCA1A\|2978; GULP1\|51454; HAUS2\|55142; |
| | HDAC4\|9759; HEPACAM2\|253012; HIPK2\|28996; HMGN4\|10473; HOXC8\|3224; HSPA6\|3310; |
| | IARS2\|55699; ICAM5\|7087; IFT122\|55764; IL12A\|3592; INSRR\|3645; IPO4\|79711; KCNH2\|3757; |
| | KCNU1\|157855; KIAA0087\|9808; KIAA0391\|9692; KIAA1328\|57536; KIAA1598\|57698; |
| | KIAA1919\|91749; KIFAP3\|22920; KRT4\|3851; KRT79\|338785; KRTDAP\|388533; LCN1\|3933; |
| | LGTN\|1939; LOC100192378\|100192378; LOC151162\|151162; LOC441208\|441208; LRP12\|29967; |
| | LRP1B\|53353; MAFG\|4097; MAP1B\|4131; MAP2\|4133; ME1\|4199; MED19\|219541; MESTIT1\|317751; |
| | MGC45800\|90768; MID1IP1\|58526; MMS19\|64210; MRPL37\|51253; NCAM1\|4684; NCAPD2\|9918; |
| | NEURL\|9148; NLN\|57486; NPAS3\|64067; NPHP4\|261734; NRSN2\|80023; NT5C3L\|115024; |
| | NTRK1\|4914; NTS\|4922; NUCKS1\|64710; NUP188\|23511; NXPH4\|11247; OBP2A\|29991; ODZ3\|55714; |
| | OR2W3\|343171; OSCP1\|127700; OTX1\|5013; PCDHB11\|56125; PCDHB8\|56128; PCDHGB1\|56104; |
| | PCLO\|27445; PFKM\|5213; PGLYRP3\|114771; PGLYRP4\|57115; PIK3C3\|5289; PINX1\|54984; PIP\|5304; |
| | PLAGL1\|5325; PLD5\|200150; POLR3D\|661; PPP2R2C\|5522; PRDM13\|59336; PRL\|5617; |
| | PRMT5\|10419; PRND\|23627; PRPF19\|27339; PRRT4\|401399; PRSS37\|136242; PVT1\|5820; |
| | RAB28\|9364; RAC3\|5881; RASD1\|51655; RBBP5\|5929; RBP7\|116362; RHOXF2B\|727940; |
| | RPAP1\|26015; SARS\|6301; SCD\|6319; SERPINB10\|5273; SERPINB12\|89777; SERPINI1\|5274; |
| | SH2D6\|284948; SLC2A12\|154091; SLC38A11\|151258; SLC9A3R1\|9368; SLCO3A1\|28232; |
| | SOSTDC1\|25928; SPSB4\|92369; SPTBN2\|6712; SRP54\|6729; SSRP1\|6749; STAC2\|342667; |
| | STRAP\|11171; STXBP1\|6812; SUPT16H\|11198; SUPT6H\|6830; TAF4B\|6875; TAS1R3\|83756; |
| | TBCD\|6904; TBXAS1\|6916; TCL1B\|9623; TGFBR2\|7048; TKT\|7086; TMCC2\|9911; TMEM48\|55706; |
| | TMEM5\|10329; TMEM61\|199964; TMX2\|51075; TNN\|63923; TPD52L1\|7164; TR1M16\|10626; |
| | TRIM9\|114088; TROVE2\|6738; TUBA1A\|7846; TUBGCP5\|114791; TULP3\|7289; UBE2QL1\|134111; |
| | UCHL1\|7345; UCHL5\|51377; USP13\|8975; USP5\|8078; UTP18\|51096; VDAC2\|7417; VWA5B2\|90113; |
| | XPOT\|11260; YARS\|8565; ZC3HAV1L\|92092; ZNF385D\|79750; ZNF804B\|219578 |
| Green | ADAM23\|8745; ADRA1D\|146; ALG1\|56052; ANGPT1\|284; AP2A2\|161; B4GALNT1\|2583; |
| Module | C12orf61\|283416; CES4\|51716; CLEC4G\|339390; CLSTN2\|64084; CXCL12\|6387; CYTL1\|54360; |
| | DAD1\|1603; EMP3\|2014; ENPP1\|5167; EPDR1\|54749; F13A1\|2162; F2RL2\|2151; FAM101B\|359845; |
| | FAM20C\|56975; FHL3\|2275; FOLR2\|2350; FOXL1\|2300; GALK1\|2584; GPC1\|2817; HECW1\|23072; |
| | HHIPL2\|79802; HPD\|3242; IL31RA\|133396; LGALS1\|3956; LYVE1\|10894; MFF\|56947; MRO\|83876; |
| | NAMPT\|10135; NEFL\|4747; NES\|10763; NHEDC2\|133308; NID2\|22795; NOTCH3\|4854; NPR3\|4883; |
| | NROB1\|190; NRCAM\|4897; NTRK2\|4915; PCDH12\|51294; PCOLCE2\|26577; PDGFD\|80310; |
| | PGA3\|643834; PGA5\|5222; PHOSPHO1\|162466; PLCZ1\|89869; PRSS23\|11098; RASGRP4\|115727; |
| | SLC47A1\|55244; SNX17\|9784; SOST\|50964; SPANXC\|64663; STK32B\|55351; STXBP5L\|9515; |
| | TBXA2R\|6915; THOP1\|7064; TM4SF1\|4071; TMEM26\|219623; TREML3\|340206; TREML4\|285852; |
| | TRIM16L\|147166; TRPV2\|51393; TXNRD1\|7296; UBTD1\|80019 |
| Red | AXIN28313; BRMS1L\|84312; C18orf54\|162681; C20orf177\|63939; CNTN1\|1272; DDX21\|9188; |
| Module | DLX1\|1745; DLX4\|1748; DYM\|54808; FGF19\|9965; GABRA3\|2556; GLCE\|26035; GTF2A1\|2957; |
| | HOXC5\|3222; KIF1B\|23095; KLHDC10\|23008; KPNB1\|3837; LMAN1\|3998; MAN2A1\|4124; |
| | MEP1B\|4225; MFSD11\|79157; MGC12916\|84815; NELF\|26012; NELL2\|4753; NXPH3\|11248; |
| | PAFAH1B2\|5049; PAM\|5066; PDE5A\|8654; PIGS\|94005; PRR11\|55771; RIMBP2\|23504; SLC1A5\|6510; |
| | STRN\|6801; TCF4\|6925; TMPRSS15\|5651; WLS\|79971 |
| Cyan | ABCB9\|23457; ACOT13\|55856; AGAP4\|119016; AGAP6\|414189; AGER\|177; AGXT2L2\|85007; |
| Module | AHSA2\|130872; AK3\|50808; AKR1B15\|441282; AKR1B1\|231; ALS2CL\|259173; ANAPC4\|29945; |
| | ANGEL2\|90806; ANKRD10\|55608; ANKRD22\|118932; ANO9\|338440; APLF\|200558; |
| | APOBEC3D\|140564; APOBEC3F\|200316; APOBEC3G\|60489; APOL1\|8542; APOL2\|23780; |
| | APOL4\|80832; APOL6\|80830; ARRDC2\|27106; ASB13\|79754; ATF7IP2\|80063; ATOH8\|84913; |
| | BAT1\|7919; BATF\|10538; BCL2L14\|79370; BLOC1S3\|388552; BTN2A1\|11120; C11orf66\|220004; |
| | C13orf39\|196541; C15orf58\|390637; C17orf86\|654434; C19orf66\|55337; C19orf6\|91304; |
| | C19orf71\|100128569; C1orf126\|200197; C1orf159\|54991; C1orf213\|148898; C1orf63\|57035; |
| | C20orf196\|149840; C20orf96\|140680; C22orf43\|51233; C2orf60\|129450; C2orf63\|130162; |
| | C3orf19\|51244; C3orf23\|285343; C3orf62\|375341; C4orf21\|55345; C5orf56\|441108; C6orf115\|58527; |
| | C6orf134\|79969; C6orf136\|221545; C6orf47\|57827; C6orf62\|81688; C8orf44\|56260; CALML3\|810; |
| | CARD11\|84433; CARD9\|64170; CASP9\|842; CCDC130\|81576; CCDC14\|64770; CCDC24\|149473; |
| | CCNJL\|79616; CCNL1\|57018; CCNL2\|81669; CCNT2\|905; CCRL2\|9034; CCT6P1\|643253; CD96\|10225; |
| | CDC42EP5\|148170; CDK10\|8558; CDK3\|1018; CEACAM16\|388551; CELF6\|60677; |
| | CHKB-CPT1B\|386593; CHMP4C\|92421; CIR1\|9541; CLDN15\|24146; CLEC2D\|29121; CLK1\|1195; |
| | CNKSR1\|10256; COLQ\|8292; COX7B\|1349; COX8C\|341947; CPT1B\|1375; CRB3\|92359; |
| | CROCCL1\|84809; CRTC2\|200186; CSAD\|51380; CTRL\|1506; CTU1\|90353; CYP2C8\|1558; |
| | CYP4Z1\|199974; CYTH2\|9266; CYorf15B\|84663; DCAF4L1\|285429; DCDC2B\|149069; |
| | DEDD2\|162989; DEGS2\|123099; DMTF1\|9988; DNAH1\|25981; DNASE1L2\|1775; DOK7\|285489; |
| | DOM3Z\|1797; ECHDC2\|55268; EFHD2\|79180; ELF4\|2000; ELMOD3\|84173; ENGASE\|64772; |
| | ERCC5\|2073; ETV7\|51513; FAAH2\|158584; FAAH\|2166; FAM113B\|91523; FAM122B\|159090; |
| | FAM13A\|10144; FAM166B\|730112; FAM193B\|54540; FAM200B\|285550; FAM25A\|643161; |
| | FAM25B\|100132929; FAM73B\|84895; FANCF\|2188; FBP1\|2203; FBXO46\|23403; FBXO6\|26270; |
| | FCHSD1\|89848; FER1L4\|80307; FITM1\|161247; FLJ12825\|440101; FNBP4\|23360; FOXD4L1\|200350; |
| | GAK\|2580; GBA2\|57704; GEMIN8\|54960; GGA1\|26088; GGTLC1\|92086; GK\|2710; GLTSCR1\|29998; |
| | GLYCTK\|132158; GMIP\|51291; GOLGA2B\|55592; GRIPAP1\|56850; GSDMB\|55876; HCG26\|352961; |
| | HCG27\|253018; HCG4P6\|80868; HDAC10\|83933; HDHD3\|81932; HEXDC\|284004; HIP1R\|9026; |
| | HIST1H2BL\|8340; HIST1H4C\|8364; HIST1H4J\|8363; HIST2H2AC\|8338; HLA-L\|3139; HNMT\|3176; |
| | HOXB5\|3215; HOXB7\|3217; HSH2D\|84941; HSPA1L\|3305; ID2B\|84099; IDUA\|3425; IFT27\|11020; |
| | IKBKB\|3551; INSL3\|3640; IP6K2\|51447; IRF1\|3659; KCNJ15\|3772; KIAA0907\|22889; |
| | KIAA1530\|57654; KIAA1875\|340390; KIF21B\|23046; KIF25\|3834; KLHL36\|79786; KLRA1\|10748; |
| | KRT23\|25984; LENG8\|114823; LIME1\|54923; LIPT1\|51601; LMBR1L\|55716; |
| | LOC100129637\|100129637; LOC100144604\|100144604; LOC100272146\|100272146; |
| | LOC100286793\|100286793; LOC100288778\|100288778; LOC146880\|146880; LOC221442\|221442; |
| | LOC283314\|283314; LOC284232\|284232; LOC284233\|284233; LOC284900\|284900; |
| | LOC285074\|285074; LOC285359\|285359; LOC388692\|388692; LOC391322\|391322; |
| | LOC400927\|400927; LOC401052\|401052; LOC440944\|440944; LOC642846\|642846; LOC91316\|91316; |
| | LUC7L\|55692; LY6G5B\|58496; MAGEB16\|139604; MAPK8IP3\|23162; ME3\|10873; MFAP3L\|9848; |
| | MFSD2A\|84879; MRFAP1L1\|114932; MRPS6\|64968; MSL3\|10943; MST1R\|4486; MTERFD3\|80298; |
| | MZF1\|7593; NADSYN1\|55191; NBR2\|10230; NCRNA00105\|80161; NCRNA00115\|79854; |
| | NDOR1\|27158; NFKBID\|84807; NFYA\|4800; NPAS2\|4862; NPIPL3\|23117; NR2F6\|2063; |
| | NSUN5P1\|155400; NSUN5P2\|260294; NSUN6\|221078; NUDT16P1\|152195; NUDT19\|390916; |
| | OAS1\|4938; OFD1\|8481; ORMDL1\|94101; ORMDL3\|94103; P2RY11\|5032; P4HB\|5034; PAQR6\|79957; |
| | PAR1\|145624; PARP4\|143; PATL2\|197135; PBOV1\|59351; PCF11\|51585; PDCL3\|79031; |
| | PDXDC2\|283970; PGPEP1\|54858; PIGA\|5277; PION\|54103; PIWIL3\|440822; PLA2G6\|8398; |
| | PLEKHA6\|22874; PLEKHH1\|57475; PLGLB2\|5342; PLIN5\|440503; PLXNB1\|5364; PMS1\|5378; |
| | PMS2L3\|5387; POLB\|5423; PPFIBP2\|8495; PRICKLE3\|4007; PRKD2\|25865; PRSS27\|83886; |
| | PSMB10\|5699; PSMB8\|5696; PTPN6\|5777; PYROXD2\|84795; RABL2A\|11159; RAD9A\|5883; |
| | RASGEF1C\|255426; RBCK1\|10616; RBM6\|10180; REEP6\|92840; REV1\|51455; RG9MTD3\|158234; |
| | RGPD6\|729540; RGS17\|26575; RPL32P3\|132241; RPP21\|79897; RTP2\|344892; RTP4\|64108; |
| | RWDD3\|25950; SCGB2A2\|4250; SCXB\|642658; SDCBP2\|27111; SEC31B\|25956; SEMA4D\|10507; |
| | SEPT7P2\|641977; SERINC4\|619189; SETMAR\|6419; SFRS16\|11129; SFRS17A\|8227; SH3GLB2\|56904; |
| | SHC3\|53358; SKINTL\|391037; SLC10A5\|347051; SLC1A6\|6511; SLC25A34\|284723; SLC45A3\|85414; |
| | SLC5A9\|200010; SLC6A2\|6530; SLC7A9\|11136; SMAD6\|4091; SP140L\|93349; SPDYA\|245711; |
| | SPG7\|6687; SPOCD1\|90853; SPSB3\|90864; STAG3L3\|442578; STAP2\|55620; STAT6\|6778; |
| | SYCP3\|50511; TAF1C\|9013; TBC1D3B\|414059; TBC1D3P2\|440452; TBC1D3\|729873; |
| | TCEANC\|170082; TCTE3\|6991; TECR\|9524; THUMPD2\|80745; TIA1\|7072; TMC7\|79905; |
| | TMEM51\|55092; TNFAIP2\|7127; TNK1\|8711; TOP3B\|8940; TRAPPC2\|6399; TRIM26\|7726; |
| | TRIM27\|5987; TRIM38\|10475; TRPV1\|7442; TSPAN14\|81619; TSPYL6\|388951; TTLL3\|26140; |
| | UBD\|10537; UCP3\|7352; UNC93B1\|81622; UPK3A\|7380; USF1\|7391; WASH3P\|374666; |
| | WASH7P\|653635; WDR52\|55779; WDR6\|11180; YTHDC1\|91746; ZCWPW1\|55063; ZFPM1\|161882; |
| | ZNF100\|163227; ZNF137\|7696; ZNF160\|90338; ZNF165\|7718; ZNF169\|169841; ZNF182\|7569; |
| | ZNF187\|7741; ZNF193\|7746; ZNF195\|7748; ZNF254\|9534; ZNF443\|10224; ZNF480\|147657; |
| | ZNF493\|284443; ZNF506\|440515; ZNF513\|130557; ZNF524\|147807; ZNF564\|163050; ZNF577\|84765; |
| | ZNF600\|162966; ZNF630\|57232; ZNF638\|27332; ZNF705A\|440077; ZNF708\|7562; ZNF763\|284390; |
| | ZNF799\|90576; ZNF814\|730051; ZNF823\|55552; ZNF841\|284371; ZNRD1\|30834; ZRANB2\|9406; |
| | ZRSR2\|8233; ZSCAN16\|80345; ZSCAN5B\|342933 |
| Yellow | ACCN2\|41; ADRA1B\|147; AIF1L\|83543; ANLN\|54443; ARID3A\|1820; ATP12A\|479; |
| Module | ATP6V1C2\|245973; C11orf20\|25858; C2orf62\|375307; C7orf33\|202865; C8orf31\|286122; CAPG\|822; |
| | CAST\|831; CCDC54\|84692; CDKN1C\|1028; CGA\|1081; CGB1\|114335; CLIC3\|9022; COL9A3\|1299; |
| | CSF3R\|1441; CTPS\|1503; DDB1\|1642; DISP2\|85455; DNMT3L\|29947; DUSP13\|51207; EIF4A3\|9775; |
| | EIF4E1B\|253314; ENTPD2\|954; FAM49A\|81553; FASN\|2194; FLJ43390\|646113; GBX2\|2637; |
| | GNA12\|2768; GOLGA8G\|283768; GPR32\|2854; GRAMD2 196996; HDAC5\|10014; HTRA4\|203100; |
| | IGF2BP3\|10643; KIF26A\|26153; KLRG2\|346689; L1TD1\|54596; LIN28A\|79727; LIN28B\|389421; |
| | LTBP1\|4052; MPRIP\|23164; NEBL\|10529; NLRP12\|91662; OTX2\|5015; PADI4\|23569; PCSK5\|5125; |
| | PDE6H\|5149; PEG10\|23089; PGF\|5228; PLEKHG4B\|153478; PPT2\|9374; PTPLB\|201562; |
| | PTPN21\|11099; RASA1\|5921; RPTOR\|57521; SIGLEC6\|946; SLC12A2\|6558; SLC12A3\|6559; |
| | SLC16A6\|9120; SLC22A11\|55867; SLC27A6\|28965; SLC2A14\|144195; SLC2A3\|6515; SNX24\|28966; |
| | SNX2\|6643; SORT1\|6272; SPRR3\|6707; SRP68\|6730; SUN3\|256979; TET1\|80312; TMEM104\|54868; |
| | TPPP3\|51673; TRIML1\|339976; TUBAL3\|79861; TYRO3\|7301; XAGE2\|9502; ZW10\|9183 |

### Example 5

### Analysis of Copy Number Variation

Test Method:

An analysis was performed by use of the CNV data from “SNP6 Copy Number Analysis (Gistic2)” in Broad GDAC Firehose (Level 4). CNV data for 1078 key genes selected from 400 BLCA samples were obtained, including 129 samples from stage I/II, 139 samples from stage III, and 132 samples from stage IV. For each gene, the frequency (i.e., amplification or deletion) of the sample with CNV in each phase was calculated. Taking into account the imbalance in the number of samples from different stages of bladder cancer, the frequency of the respective phase was normalized by use of Stage I/II as a baseline.

Test Results:

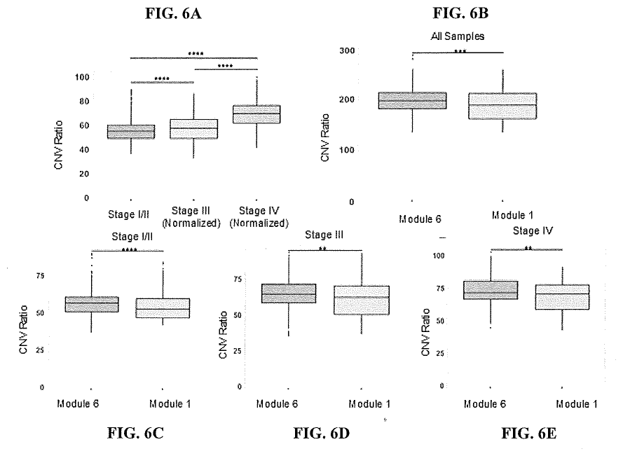

The results showed that the different stages of bladder cancer (stages I/II, III and IV) showed significantly different CNV frequencies, and the CNV increased significantly with the progression of bladder cancer (see FIG. 6A). This result means that the copy number abnormalities may contribute to the progression of bladder cancer. Meanwhile, the CNVs of the genes in the blue module and the cyan module (see Module 6 and Module 1 in FIG. 5B) in Example 4 were examined (see Table 7), which were most positively and negatively correlated with different stages of bladder cancer, respectively. It was found that in all the samples or in various stages of the BLCA patients, the blue module (where all genes are risk effective genes) showed greater CNV ratios than the cyan module (most of which (i.e., 93%) genes were protective effective genes) (see FIGS. 6B-6E). Of those, FIG. 6A shows a comparison of CNV ratios in different stages of bladder cancer. FIGS. 6B-6E show the comparison of CNV ratios for the blue and cyan modules as a whole and for Stages I/II, III and IV; where *p value<0.05; **: p value<0.01; ***: p value<0.001; ****: p value<0.0001, as detected by double-sided Wilcoxon rank sum test. The results indicate that copy number variation is an important factor affecting different stages (i.e., progression) of bladder cancer, and affects different functional gene modules at different levels.

TABLE 7
CNVs of Genes in Cyan and Blue Modules

| | Stage | | | | | Stage | | | |
|---|---|---|---|---|---|---|---|---|---|
| | I + | | | | | I + | | | |
| | Stage | Stage | Stage | | | Stage | Stage | Stage | |
| | II | III | IV | All | | II | III | IV | All |
| Cyan Module | (129) | (139) | (132) | Stages | Blue Module | (129) | (139) | (132) | Stages |
| ABCB9\|23457 | 53 | 53 | 60 | 166 | ABCC9\|10060 | 59 | 58 | 60 | 177 |
| ACOT13\|55856 | 56 | 65 | 74 | 195 | ACVR1\|90 | 46 | 50 | 63 | 159 |
| AGAP4\|119016 | 57 | 62 | 64 | 183 | ADAMTS12\|81792 | 69 | 81 | 77 | 227 |
| AGAP6\|414189 | 57 | 62 | 65 | 184 | ADAMTS16\|170690 | 74 | 87 | 80 | 241 |
| AGER\|177 | 51 | 61 | 72 | 184 | ADAMTS18\|170692 | 54 | 73 | 74 | 201 |
| AGXT2L2\|85007 | 63 | 78 | 82 | 223 | ADAMTSL1\|92949 | 83 | 95 | 83 | 261 |
| AHSA2\|130872 | 45 | 51 | 69 | 165 | ADCY7\|113 | 51 | 67 | 73 | 191 |
| AK3\|50808 | 80 | 95 | 82 | 257 | AHNAK\|79026 | 51 | 54 | 69 | 174 |
| AKR1B1\|231 | 52 | 55 | 73 | 180 | ALAS2\|212 | 49 | 44 | 46 | 139 |
| AKR1B15\|441282 | 52 | 56 | 73 | 181 | ALDHIL2\|160428 | 54 | 51 | 57 | 162 |
| ALS2CL\|259173 | 61 | 67 | 81 | 209 | ANPEP\|290 | 55 | 64 | 64 | 183 |
| ANAPC4\|29945 | 56 | 62 | 74 | 192 | ANXA1\|301 | 71 | 87 | 75 | 233 |
| ANGEL2\|90806 | 64 | 53 | 71 | 188 | ANXA2\|302 | 51 | 66 | 61 | 178 |
| ANKRD10\|55608 | 58 | 76 | 73 | 207 | ANXA5\|308 | 55 | 63 | 68 | 186 |
| ANKRD22\|118932 | 63 | 58 | 74 | 195 | ARCN1\|372 | 62 | 64 | 70 | 196 |
| ANO9\|338440 | 70 | 71 | 92 | 233 | ARHGAP29\|9411 | 46 | 38 | 64 | 148 |
| APLF\|200558 | 44 | 51 | 67 | 162 | ARL10\|285598 | 62 | 78 | 82 | 222 |
| APOBEC3D\|140564 | 69 | 78 | 77 | 224 | ARL4C\|10123 | 67 | 70 | 81 | 218 |
| APOBEC3F\|200316 | 69 | 78 | 77 | 224 | ARMC9\|80210 | 67 | 72 | 79 | 218 |
| APOBEC3G\|60489 | 69 | 78 | 77 | 224 | ARSI\|340075 | 59 | 72 | 82 | 213 |
| APOL1\|8542 | 70 | 78 | 73 | 221 | ATF6\|22926 | 72 | 63 | 85 | 220 |
| APOL2\|23780 | 70 | 78 | 73 | 221 | ATG9A\|79065 | 62 | 68 | 81 | 211 |
| APOL4\|80832 | 69 | 78 | 73 | 220 | ATP2B4\|493 | 62 | 54 | 70 | 186 |
| APOL6\|80830 | 69 | 77 | 74 | 220 | ATP6V0D1\|9114 | 58 | 72 | 76 | 206 |
| ARRDC2\|27106 | 45 | 64 | 70 | 179 | ATP6V1B2\|526 | 90 | 91 | 100 | 281 |
| ASB13\|79754 | 67 | 75 | 76 | 218 | BACE1\|23621 | 62 | 63 | 70 | 195 |
| ATF7IP2\|80063 | 52 | 73 | 80 | 205 | BAIAP2\|10458 | 54 | 72 | 80 | 206 |
| ATOH8\|84913 | 45 | 49 | 62 | 156 | BARX2\|8538 | 63 | 67 | 70 | 200 |
| BAT1\|7919 | 51 | 61 | 72 | 184 | C13orf15\|28984 | 59 | 73 | 79 | 211 |
| BATF\|10538 | 53 | 70 | 67 | 190 | C13orf33\|84935 | 58 | 69 | 71 | 198 |
| BCL2L14\|79370 | 60 | 63 | 62 | 185 | C14orf37\|145407 | 55 | 68 | 71 | 194 |
| BLOC1S3\|388552 | 58 | 75 | 80 | 213 | C15orf38\|348110 | 56 | 65 | 64 | 185 |
| BTN2A1\|11120 | 55 | 65 | 72 | 192 | C15orf54\|400360 | 56 | 65 | 68 | 189 |
| C11orf66\|220004 | 51 | 54 | 69 | 174 | C17orf51\|339263 | 60 | 75 | 82 | 217 |
| C13orf39\|196541 | 62 | 76 | 72 | 210 | C19orf59\|199675 | 49 | 68 | 79 | 196 |
| C15orf58\|390637 | 55 | 65 | 64 | 184 | C5orf62\|85027 | 60 | 73 | 82 | 215 |
| C19orf6\|91304 | 51 | 68 | 78 | 197 | C6orf138\|442213 | 53 | 57 | 73 | 183 |
| C19orf66\|55337 | 50 | 67 | 78 | 195 | CALU\|813 | 53 | 52 | 72 | 177 |
| C19orf71\|100128569 | 51 | 69 | 79 | 199 | CAPN2\|824 | 65 | 54 | 73 | 192 |
| C1orf159\|54991 | 52 | 52 | 58 | 162 | CBLN4\|140689 | 77 | 81 | 92 | 250 |
| C1orf213\|148898 | 50 | 48 | 56 | 154 | CCDC8\|83987 | 56 | 73 | 82 | 211 |
| C1orf63\|57035 | 48 | 46 | 56 | 150 | CCDC80\|151887 | 56 | 71 | 78 | 205 |
| C20orf196\|149840 | 68 | 78 | 86 | 232 | CD109\|135228 | 61 | 63 | 74 | 198 |
| C20orf96\|140680 | 69 | 78 | 85 | 232 | CD276\|80381 | 55 | 64 | 62 | 181 |
| C22orf43\|51233 | 66 | 76 | 77 | 219 | CD300LG\|146894 | 48 | 62 | 71 | 181 |
| C2orf60\|129450 | 51 | 57 | 68 | 176 | CDK6\|1021 | 56 | 58 | 69 | 183 |
| C2orf63\|130162 | 46 | 50 | 66 | 162 | CERCAM\|51148 | 67 | 85 | 70 | 222 |
| C3orf19\|51244 | 62 | 71 | 89 | 222 | CHPF2\|54480 | 52 | 56 | 70 | 178 |
| C3orf23\|285343 | 61 | 66 | 82 | 209 | CHRNA1\|1134 | 45 | 51 | 63 | 159 |
| C3orf62\|375341 | 62 | 71 | 85 | 218 | CHSY1\|22856 | 57 | 69 | 67 | 193 |
| C4orf21\|55345 | 54 | 61 | 67 | 182 | CIDEC\|63924 | 65 | 73 | 90 | 228 |
| C5orf56\|441108 | 60 | 73 | 80 | 213 | CKAP4\|10970 | 55 | 51 | 58 | 164 |
| C6orf115\|58527 | 60 | 71 | 83 | 214 | CKMT2\|1160 | 62 | 77 | 84 | 223 |
| C6orf134\|79969 | 52 | 62 | 71 | 185 | CLEC11A\|6320 | 59 | 74 | 84 | 217 |
| C6orf136\|221545 | 52 | 62 | 71 | 185 | CNIH\|10175 | 51 | 67 | 70 | 188 |
| C6orf47\|57827 | 51 | 61 | 72 | 184 | CNN3\|1266 | 43 | 39 | 63 | 145 |
| C6orf62\|81688 | 54 | 65 | 74 | 193 | CNTNAP3\|79937 | 79 | 91 | 84 | 254 |
| C8orf44\|56260 | 76 | 79 | 85 | 240 | COL18A1\|80781 | 63 | 70 | 81 | 214 |
| CALML3\|810 | 67 | 75 | 76 | 218 | COL4A2\|1284 | 59 | 76 | 74 | 209 |
| CARD11\|84433 | 53 | 72 | 84 | 209 | COL5A1\|1289 | 67 | 82 | 73 | 222 |
| CASP9\|842 | 51 | 50 | 55 | 156 | COL5A3\|50509 | 50 | 68 | 77 | 195 |
| CCDC130\|81576 | 48 | 65 | 69 | 182 | COL6A1\|1291 | 63 | 70 | 80 | 213 |
| CCDC14\|64770 | 60 | 70 | 82 | 212 | COPS8\|10920 | 67 | 69 | 80 | 216 |
| CCDC24\|149473 | 46 | 41 | 53 | 140 | COPZ2\|51226 | 47 | 62 | 77 | 186 |
| CCNJL\|79616 | 61 | 73 | 82 | 216 | CPXM1\|56265 | 69 | 78 | 85 | 232 |
| CCNL1\|57018 | 64 | 74 | 81 | 219 | CRTAP\|10491 | 59 | 65 | 85 | 209 |
| CCNL2\|81669 | 52 | 52 | 58 | 162 | CSGALNACT2\|55454 | 60 | 63 | 61 | 184 |
| CCNT2\|905 | 47 | 47 | 58 | 152 | CSPG4\|1464 | 55 | 65 | 62 | 182 |
| CCRL2\|9034 | 62 | 66 | 82 | 210 | CTNNB1\|1499 | 61 | 66 | 84 | 211 |
| CCT6P1\|643253 | 55 | 64 | 71 | 190 | CUBN\|8029 | 65 | 68 | 70 | 203 |
| CD96\|10225 | 55 | 72 | 79 | 206 | CXCR1\|3577 | 62 | 68 | 79 | 209 |
| CDC42EP5\|148170 | 60 | 72 | 81 | 213 | CYTH3\|9265 | 53 | 72 | 86 | 211 |
| CDK10\|8558 | 57 | 70 | 73 | 200 | DIRAS3\|9077 | 44 | 37 | 54 | 135 |
| CDK3\|1018 | 56 | 71 | 79 | 206 | DNAJB4\|11080 | 45 | 40 | 56 | 141 |
| CEACAM16\|388551 | 57 | 74 | 80 | 211 | DNM3\|26052 | 64 | 63 | 81 | 208 |
| CELF6\|60677 | 54 | 63 | 61 | 178 | DSEL\|92126 | 74 | 83 | 73 | 230 |
| CHMP4C\|92421 | 79 | 83 | 89 | 251 | DUSP14\|11072 | 52 | 64 | 71 | 187 |
| CIR1\|9541 | 45 | 52 | 62 | 159 | DYRK3\|8444 | 62 | 53 | 71 | 186 |
| CLDN15\|24146 | 51 | 59 | 70 | 180 | ECM1\|1893 | 69 | 62 | 82 | 213 |
| CLEC2D\|29121 | 60 | 61 | 60 | 181 | EDNRA\|1909 | 58 | 66 | 72 | 196 |
| CLK1\|1195 | 51 | 56 | 69 | 176 | EFCAB1\|79645 | 78 | 79 | 86 | 243 |
| CNKSR1\|10256 | 47 | 49 | 55 | 151 | EHBP1\|23301 | 46 | 52 | 70 | 168 |
| COLQ\|8292 | 62 | 70 | 88 | 220 | EIF2AK4\|440275 | 57 | 65 | 67 | 189 |
| COX7B\|1349 | 47 | 44 | 44 | 135 | EMP1\|2012 | 59 | 63 | 61 | 183 |
| COX8C\|341947 | 55 | 72 | 63 | 190 | ENDOD1\|23052 | 58 | 61 | 67 | 186 |
| CPT1B\|1375 | 68 | 80 | 82 | 230 | EPHB3\|2049 | 66 | 77 | 81 | 224 |
| CRB3\|92359 | 50 | 69 | 79 | 198 | EPHB4\|2050 | 52 | 60 | 69 | 181 |
| CRTC2\|200186 | 69 | 60 | 79 | 208 | ERC1\|23085 | 60 | 62 | 62 | 184 |
| CSAD\|51380 | 59 | 46 | 51 | 156 | ESYT2\|57488 | 54 | 61 | 70 | 185 |
| CTRL\|1506 | 59 | 73 | 76 | 208 | ETF1\|2107 | 59 | 75 | 79 | 213 |
| CTU1\|90353 | 59 | 74 | 83 | 216 | EXTL3\|2137 | 88 | 92 | 101 | 281 |
| CYP2C8\|1558 | 62 | 58 | 74 | 194 | EYS\|346007 | 60 | 61 | 79 | 200 |
| CYP4Z1\|199974 | 46 | 41 | 50 | 137 | F10\|2159 | 58 | 74 | 75 | 207 |
| CYTH2\|9266 | 60 | 70 | 84 | 214 | FAM126A\|84668 | 51 | 67 | 86 | 204 |
| DCAF4LI\|285429 | 55 | 56 | 73 | 184 | FAM129B\|64855 | 67 | 84 | 70 | 221 |
| DCDC2B\|149069 | 44 | 44 | 53 | 141 | FAM168A\|23201 | 58 | 61 | 73 | 192 |
| DEDD2\|162989 | 58 | 74 | 77 | 209 | FAM180A\|389558 | 52 | 55 | 72 | 179 |
| DEGS2\|123099 | 57 | 69 | 65 | 191 | FAM27B\|100133121 | 79 | 91 | 84 | 254 |
| DMTF1\|9988 | 53 | 58 | 69 | 180 | FAM43A\|131583 | 65 | 75 | 80 | 220 |
| DNAH1\|25981 | 60 | 71 | 83 | 214 | FJX1\|24147 | 62 | 64 | 85 | 211 |
| DNASE1L2\|1775 | 56 | 75 | 80 | 211 | FKBP10\|60681 | 48 | 61 | 71 | 180 |
| DOK7\|285489 | 60 | 59 | 75 | 194 | FKBP14\|55033 | 52 | 64 | 83 | 199 |
| DOM3Z\|1797 | 51 | 61 | 72 | 184 | FKBP9\|11328 | 55 | 65 | 79 | 199 |
| ECHDC2\|55268 | 47 | 39 | 52 | 138 | FLRT2\|23768 | 53 | 71 | 63 | 187 |
| EFHD2\|79180 | 50 | 50 | 55 | 155 | FN1\|2335 | 62 | 68 | 79 | 209 |
| ELF4\|2000 | 46 | 48 | 48 | 142 | FUT11\|170384 | 57 | 60 | 69 | 186 |
| ELMOD3\|84173 | 43 | 48 | 63 | 154 | GABRG1\|2565 | 54 | 57 | 67 | 178 |
| ENGASE\|64772 | 56 | 72 | 80 | 208 | GANAB\|23193 | 51 | 55 | 69 | 175 |
| ERCC5\|2073 | 62 | 76 | 72 | 210 | GAS7\|8522 | 72 | 86 | 89 | 247 |
| ETV7\|51513 | 53 | 60 | 71 | 184 | GFPT2\|9945 | 61 | 76 | 83 | 220 |
| FAAH\|2166 | 46 | 41 | 50 | 137 | GJA1\|2697 | 59 | 67 | 83 | 209 |
| FAAH2\|158584 | 49 | 44 | 46 | 139 | GLI2\|2736 | 42 | 46 | 54 | 142 |
| FAM113B\|91523 | 54 | 47 | 55 | 156 | GLT25D1\|79709 | 46 | 64 | 70 | 180 |
| FAM122B\|159090 | 46 | 47 | 49 | 142 | GPX8\|493869 | 60 | 81 | 85 | 226 |
| FAM13A\|10144 | 53 | 60 | 67 | 180 | GRIK2\|2898 | 63 | 65 | 83 | 211 |
| FAM166B\|730112 | 75 | 86 | 84 | 245 | GUCY1B3\|2983 | 63 | 66 | 72 | 201 |
| FAM193B\|54540 | 63 | 78 | 82 | 223 | GXYLT2\|727936 | 58 | 71 | 79 | 208 |
| FAM200B\|285550 | 57 | 62 | 73 | 192 | HDLBP\|3069 | 67 | 69 | 79 | 215 |
| FAM25A\|643161 | 60 | 58 | 76 | 194 | HEYL\|26508 | 47 | 43 | 59 | 149 |
| FAM25B\|100132929 | 57 | 62 | 64 | 183 | IGF1\|3479 | 53 | 54 | 57 | 164 |
| FAM73B\|84895 | 66 | 85 | 69 | 220 | IGF2R\|3482 | 61 | 74 | 86 | 221 |
| FANCF\|2188 | 67 | 68 | 91 | 226 | IGFL2\|147920 | 57 | 73 | 82 | 212 |
| FBP1\|2203 | 74 | 86 | 73 | 233 | IMPDH1\|3614 | 55 | 52 | 72 | 179 |
| FBXO46\|23403 | 59 | 74 | 81 | 214 | INHBB\|3625 | 42 | 46 | 54 | 142 |
| FBXO6\|26270 | 50 | 50 | 56 | 156 | IPO11\|51194 | 63 | 76 | 88 | 227 |
| FCHSD1\|89848 | 59 | 73 | 81 | 213 | IQGAP1\|8826 | 55 | 65 | 64 | 184 |
| FER1L4\|80307 | 77 | 83 | 93 | 253 | ITFG1\|81533 | 54 | 68 | 75 | 197 |
| FITM1\|161247 | 53 | 70 | 70 | 193 | ITGA1\|3672 | 60 | 76 | 83 | 219 |
| FNBP4\|23360 | 61 | 62 | 83 | 206 | ITGB8\|3696 | 51 | 67 | 86 | 204 |
| FOXD4L1\|200350 | 43 | 46 | 55 | 144 | JAG1\|182 | 73 | 77 | 87 | 237 |
| GAK\|2580 | 65 | 61 | 76 | 202 | JDP2\|122953 | 53 | 69 | 67 | 189 |
| GBA2\|57704 | 74 | 86 | 83 | 243 | KANK4\|163782 | 44 | 38 | 56 | 138 |
| GEMIN8\|54960 | 52 | 50 | 50 | 152 | KCNE4\|23704 | 63 | 69 | 81 | 213 |
| GGA1\|26088 | 70 | 77 | 75 | 222 | KCTD20\|222658 | 54 | 60 | 71 | 185 |
| GGTLC1\|92086 | 73 | 81 | 86 | 240 | KCTD4\|386618 | 58 | 72 | 78 | 208 |
| GK\|2710 | 49 | 49 | 50 | 148 | KDELC2\|143888 | 61 | 64 | 70 | 195 |
| GLTSCR1\|29998 | 57 | 70 | 83 | 210 | KDSR\|2531 | 74 | 82 | 72 | 228 |
| GLYCTK\|132158 | 59 | 70 | 83 | 212 | KIAA0090\|23065 | 49 | 49 | 55 | 153 |
| GMIP\|51291 | 46 | 67 | 72 | 185 | LAMC1\|3915 | 60 | 59 | 76 | 195 |
| GOLGA2B\|55592 | 53 | 52 | 58 | 163 | LDLR\|3949 | 50 | 69 | 74 | 193 |
| GRIPAP1\|56850 | 48 | 52 | 51 | 151 | LDLRAD3\|143458 | 63 | 65 | 87 | 215 |
| GSDMB\|55876 | 51 | 64 | 70 | 185 | LEPROT\|54741 | 46 | 37 | 55 | 138 |
| HCG27\|253018 | 52 | 63 | 71 | 186 | LHFP\|10186 | 58 | 72 | 80 | 210 |
| HDAC10\|83933 | 68 | 80 | 82 | 230 | LOX\|4015 | 61 | 76 | 82 | 219 |
| HDHD3\|81932 | 68 | 86 | 68 | 222 | LRP1\|4035 | 56 | 47 | 52 | 155 |
| HEXDC\|284004 | 55 | 72 | 80 | 207 | MAP1A\|4130 | 57 | 63 | 64 | 184 |
| HIP1R\|9026 | 52 | 53 | 60 | 165 | MAP2K1\|5604 | 55 | 62 | 62 | 179 |
| HIST1H2BL\|8340 | 53 | 64 | 72 | 189 | MAP7D1\|55700 | 47 | 42 | 55 | 144 |
| HIST1H4C\|8364 | 54 | 65 | 72 | 191 | MAP7D3\|79649 | 46 | 47 | 49 | 142 |
| HIST1H4J\|8363 | 54 | 64 | 72 | 190 | MARVELD1\|83742 | 62 | 59 | 73 | 194 |
| HIST2H2AC\|8338 | 65 | 58 | 79 | 202 | MBOAT2\|129642 | 52 | 51 | 70 | 173 |
| HNMT\|3176 | 46 | 48 | 57 | 151 | MDGA2\|161357 | 53 | 67 | 72 | 192 |
| HOXB5\|3215 | 48 | 64 | 78 | 190 | MMP16\|4325 | 79 | 84 | 91 | 254 |
| HOXB7\|3217 | 48 | 64 | 78 | 190 | MT1A\|4489 | 55 | 69 | 69 | 193 |
| HSH2D\|84941 | 50 | 66 | 68 | 184 | MTMR2\|8898 | 59 | 61 | 68 | 188 |
| HSPA1L\|3305 | 51 | 61 | 72 | 184 | MXRA7\|439921 | 55 | 71 | 79 | 205 |
| IDUA\|3425 | 65 | 61 | 76 | 202 | MYADM\|91663 | 59 | 74 | 81 | 214 |
| IFT27\|11020 | 70 | 77 | 77 | 224 | MYH10\|4628 | 71 | 85 | 87 | 243 |
| IKBKB\|3551 | 85 | 81 | 93 | 259 | MYO5A\|4644 | 57 | 65 | 64 | 186 |
| INSL3\|3640 | 45 | 64 | 70 | 179 | MYO9A\|4649 | 55 | 63 | 61 | 179 |
| IP6K2\|51447 | 61 | 72 | 84 | 217 | NAV3\|89795 | 57 | 51 | 60 | 168 |
| IRF1\|3659 | 60 | 73 | 80 | 213 | NBAS\|51594 | 52 | 52 | 69 | 173 |
| KCNJ15\|3772 | 59 | 69 | 79 | 207 | NG\|4803 | 42 | 42 | 63 | 147 |
| KIAA0907\|22889 | 66 | 63 | 78 | 207 | NPC1\|4864 | 65 | 71 | 72 | 208 |
| KIAA1530\|57654 | 65 | 63 | 75 | 203 | NUDT11\|55190 | 48 | 49 | 51 | 148 |
| KIAA1875\|340390 | 81 | 93 | 86 | 260 | OLFML2B\|25903 | 72 | 63 | 85 | 220 |
| KIF21B\|23046 | 61 | 55 | 68 | 184 | OSBPL10\|114884 | 60 | 66 | 85 | 211 |
| KIF25\|3834 | 61 | 73 | 88 | 222 | PAPPA\|5069 | 68 | 85 | 69 | 222 |
| KLHL36\|79786 | 56 | 69 | 74 | 199 | PCDHB10\|56126 | 59 | 73 | 82 | 214 |
| KLRA1\|10748 | 60 | 61 | 61 | 182 | PCDHB12\|56124 | 59 | 73 | 82 | 214 |
| KRT23\|25984 | 48 | 64 | 70 | 182 | PCDHB7\|56129 | 59 | 73 | 81 | 213 |
| LENG8\|114823 | 60 | 72 | 81 | 213 | PCDHGA1\|56114 | 59 | 73 | 81 | 213 |
| LIME1\|54923 | 76 | 79 | 92 | 247 | PCDHGA2\|56113 | 59 | 73 | 81 | 213 |
| LIPT1\|51601 | 48 | 47 | 59 | 154 | PCDHGA3\|56112 | 59 | 73 | 81 | 213 |
| LMBR1L\|55716 | 53 | 46 | 52 | 151 | PCDHGC3\|5098 | 59 | 73 | 81 | 213 |
| LOC100129637\|100129637 | 57 | 68 | 73 | 198 | PDGFC\|56034 | 62 | 66 | 72 | 200 |
| LOC221442\|221442 | 54 | 60 | 73 | 187 | PDGFRA\|5156 | 51 | 53 | 63 | 167 |
| LUC7L\|55692 | 58 | 74 | 77 | 209 | PDGFRB\|5159 | 59 | 72 | 82 | 213 |
| LY6G5B\|58496 | 51 | 61 | 72 | 184 | PDIA6\|10130 | 52 | 51 | 70 | 173 |
| MAGEB16\|139604 | 48 | 50 | 51 | 149 | PDLIM2\|64236 | 91 | 93 | 104 | 288 |
| MAPK8IP3\|23162 | 57 | 75 | 80 | 212 | PGM3\|5238 | 62 | 62 | 81 | 205 |
| ME3\|10873 | 59 | 62 | 66 | 187 | PITX3\|5309 | 63 | 58 | 72 | 193 |
| MFAP3L\|9848 | 62 | 67 | 73 | 202 | PPEF1\|5475 | 52 | 51 | 51 | 154 |
| MFSD2A\|84879 | 49 | 42 | 59 | 150 | PPP2R3A\|5523 | 63 | 71 | 82 | 216 |
| MRFAPIL1\|114932 | 58 | 59 | 75 | 192 | PRKAR2A\|5576 | 62 | 72 | 84 | 218 |
| MRPS6\|64968 | 59 | 67 | 79 | 205 | PRNP\|5621 | 68 | 79 | 85 | 232 |
| MSL3\|19943 | 52 | 50 | 50 | 152 | PTPN14\|5784 | 65 | 53 | 70 | 188 |
| MST1R\|4486 | 61 | 70 | 85 | 216 | PTPRG\|5793 | 58 | 71 | 83 | 212 |
| MTERFD3\|80298 | 55 | 51 | 57 | 163 | RAB5C\|5878 | 48 | 60 | 71 | 179 |
| MZF1\|7593 | 60 | 73 | 82 | 215 | RAPGEF5\|9771 | 50 | 67 | 86 | 203 |
| NADSYN1\|55191 | 58 | 63 | 71 | 192 | RASAL2\|9462 | 62 | 61 | 77 | 200 |
| NBR2\|10230 | 48 | 60 | 72 | 180 | RBMS3\|27303 | 61 | 66 | 84 | 211 |
| NCRNA00115\|79854 | 52 | 52 | 58 | 162 | RCAN1\|1827 | 60 | 67 | 78 | 205 |
| NDOR1\|27158 | 67 | 85 | 73 | 225 | RDX\|5962 | 61 | 63 | 70 | 194 |
| NFKBID\|84807 | 59 | 72 | 78 | 209 | RNF217\|154214 | 59 | 66 | 83 | 208 |
| NFYA\|4800 | 54 | 59 | 73 | 186 | RNF26\|79102 | 62 | 63 | 70 | 195 |
| NPAS2\|4862 | 47 | 45 | 59 | 151 | RTTN\|25914 | 72 | 83 | 75 | 230 |
| NR2F6\|2063 | 47 | 65 | 70 | 182 | RUNX2\|860 | 54 | 62 | 74 | 190 |
| NSUN5P1\|155400 | 52 | 60 | 73 | 185 | SAMD8\|142891 | 57 | 60 | 71 | 188 |
| NSUN5P2\|260294 | 53 | 60 | 73 | 186 | SC65\|10609 | 48 | 61 | 71 | 180 |
| NSUN6\|221078 | 62 | 68 | 69 | 199 | SCEL\|8796 | 59 | 72 | 73 | 204 |
| NGDT16P1\|152195 | 63 | 70 | 82 | 215 | SCRN1\|9805 | 52 | 64 | 83 | 199 |
| NUDT19\|390916 | 59 | 71 | 80 | 210 | SEC23A\|10484 | 56 | 66 | 72 | 194 |
| OAS1\|4938 | 53 | 52 | 60 | 165 | SEPT7\|989 | 54 | 63 | 79 | 196 |
| OFD1\|8481 | 52 | 50 | 50 | 152 | SERINC1\|57515 | 59 | 66 | 83 | 208 |
| ORMDL1\|94101 | 46 | 56 | 64 | 166 | SERPINF1\|5176 | 74 | 84 | 90 | 248 |
| ORMDL3\|94103 | 51 | 64 | 70 | 185 | SGCB\|6443 | 51 | 53 | 63 | 167 |
| P2RY11\|5032 | 50 | 67 | 78 | 195 | SGTB\|54557 | 65 | 78 | 88 | 231 |
| P4HB\|5034 | 54 | 73 | 80 | 207 | SHC4\|399694 | 58 | 65 | 65 | 188 |
| PAQR6\|79957 | 66 | 62 | 79 | 207 | SIDT2\|51092 | 62 | 64 | 70 | 196 |
| PARP4\|143 | 59 | 67 | 73 | 199 | SLC13A5\|284111 | 73 | 84 | 90 | 247 |
| PATL2\|197135 | 57 | 64 | 64 | 185 | SLC45A1\|50651 | 50 | 52 | 58 | 160 |
| PBOV1\|59351 | 60 | 71 | 83 | 214 | SNAI2\|6591 | 78 | 79 | 86 | 243 |
| PCF11\|51585 | 56 | 59 | 65 | 180 | SORBS3\|10174 | 91 | 93 | 104 | 288 |
| PDCL3\|79031 | 46 | 46 | 57 | 149 | SPOCK1\|6695 | 60 | 75 | 79 | 214 |
| PGPEP1\|54858 | 46 | 65 | 70 | 181 | SRPX\|8406 | 49 | 48 | 51 | 148 |
| PIGA\|5277 | 52 | 50 | 50 | 152 | STT3A\|3703 | 61 | 65 | 69 | 195 |
| PION\|54103 | 52 | 58 | 71 | 181 | STX5\|6811 | 52 | 54 | 69 | 175 |
| PIWIL3\|440822 | 65 | 77 | 76 | 218 | STYX\|6815 | 51 | 68 | 68 | 187 |
| PLA2G6\|8398 | 69 | 78 | 76 | 223 | SULF2\|55959 | 78 | 83 | 92 | 253 |
| PLEKHA6\|22874 | 63 | 54 | 73 | 190 | SUMF2\|25870 | 56 | 69 | 75 | 200 |
| PLEKHH1\|57475 | 57 | 69 | 70 | 196 | SVEP1\|79987 | 70 | 89 | 70 | 229 |
| PLGLB2\|5342 | 44 | 50 | 62 | 156 | SYDE1\|85360 | 48 | 65 | 69 | 182 |
| PLIN5\|440503 | 51 | 69 | 79 | 199 | TAF13\|6884 | 38 | 40 | 62 | 140 |
| PLXNB1\|5364 | 61 | 71 | 83 | 215 | TBC1D16\|125058 | 55 | 72 | 80 | 207 |
| PMS1\|5378 | 46 | 56 | 64 | 166 | TEAD4\|7004 | 61 | 64 | 61 | 186 |
| PMS2L3\|5387 | 52 | 60 | 74 | 186 | TEX2\|55852 | 54 | 71 | 84 | 209 |
| POLB\|5423 | 84 | 81 | 93 | 258 | TGFB1\|7045 | 62 | 74 | 80 | 216 |
| PPFIBP2\|8495 | 74 | 71 | 92 | 237 | THBS3\|7059 | 67 | 60 | 78 | 205 |
| PRICKLE3\|4007 | 48 | 52 | 51 | 151 | TMEM109\|79073 | 52 | 55 | 68 | 175 |
| PRKD2\|25865 | 57 | 69 | 82 | 208 | TMEM158\|25907 | 61 | 66 | 82 | 209 |
| PRSS27\|83886 | 56 | 75 | 80 | 211 | TMEM17\|200728 | 45 | 52 | 70 | 167 |
| PSMB10\|5699 | 59 | 73 | 76 | 208 | TMTC1\|83857 | 58 | 62 | 57 | 177 |
| PSMB8\|5696 | 51 | 61 | 72 | 184 | TNFAIP8L3\|388121 | 56 | 64 | 66 | 186 |
| PTPN6\|5777 | 60 | 62 | 59 | 181 | TNFRSF6B\|8771 | 76 | 79 | 92 | 247 |
| PYROXD2\|84795 | 62 | 59 | 73 | 194 | TOX4\|9878 | 52 | 68 | 74 | 194 |
| RABL2A\|11159 | 43 | 46 | 55 | 144 | TPST1\|8460 | 54 | 64 | 72 | 190 |
| RAD9A\|5883 | 56 | 57 | 69 | 182 | TRAM2\|9697 | 54 | 57 | 72 | 183 |
| RASGEF1C\|255426 | 61 | 76 | 82 | 219 | TSPAN9\|10867 | 63 | 64 | 61 | 188 |
| RBCK1\|10616 | 69 | 78 | 85 | 232 | TWIST2\|117581 | 66 | 69 | 77 | 212 |
| RBM6\|10180 | 62 | 70 | 85 | 217 | UACA\|55075 | 54 | 64 | 61 | 179 |
| REEP6\|92840 | 51 | 69 | 81 | 201 | VIM\|7431 | 65 | 68 | 70 | 203 |
| REV1\|51455 | 48 | 46 | 60 | 154 | VKORC1\|79001 | 47 | 64 | 73 | 184 |
| RG9MTD3\|158234 | 71 | 87 | 83 | 241 | WWTR1\|25937 | 66 | 74 | 83 | 223 |
| RGPD6\|729540 | 43 | 49 | 56 | 148 | ZNF474\|133923 | 61 | 76 | 82 | 219 |
| RGS17\|26575 | 58 | 72 | 85 | 215 | ZNF532\|55205 | 73 | 79 | 74 | 226 |
| RPL32P3\|132241 | 61 | 71 | 82 | 214 | ANXA2P1\|303 | 0 | 0 | 0 | 0 |
| RPP21\|79897 | 51 | 61 | 70 | 182 | ANXA2P2\|304 | 0 | 0 | 0 | 0 |
| RTP2\|344S92 | 67 | 77 | 79 | 223 | C6orf72\|116254 | 0 | 0 | 0 | 0 |
| RTP4\|64108 | 67 | 76 | 80 | 223 | FLJ42709\|441094 | 0 | 0 | 0 | 0 |
| RWDD3\|25950 | 43 | 40 | 65 | 148 | LOC100216001\|100216001 | 0 | 0 | 0 | 0 |
| SCGB2A2\|4250 | 51 | 53 | 70 | 174 | LOC338588\|338588 | 0 | 0 | 0 | 0 |
| SCXB\|642658 | 81 | 93 | 86 | 260 | MGC4473\|79100 | 0 | 0 | 0 | 0 |
| SDCBP2\|27111 | 69 | 78 | 85 | 232 | | | | | |
| SEC31B\|25956 | 64 | 59 | 73 | 196 | | | | | |
| SEMA4D\|10507 | 73 | 85 | 72 | 230 | | | | | |
| SEPT7P2\|641977 | 55 | 63 | 79 | 197 | | | | | |
| SERINC4\|619189 | 57 | 63 | 64 | 184 | | | | | |
| SETMAR\|6419 | 63 | 72 | 85 | 220 | | | | | |
| SFRS16\|11129 | 58 | 74 | 79 | 211 | | | | | |
| SFRS17A\|8227 | 56 | 54 | 53 | 163 | | | | | |
| SH3GLB2\|56904 | 66 | 85 | 69 | 220 | | | | | |
| SHC3\|53358 | 73 | 84 | 72 | 229 | | | | | |
| SKINTL\|391037 | 46 | 39 | 53 | 138 | | | | | |
| SLC10A5\|347051 | 79 | 83 | 89 | 251 | | | | | |
| SLC1A6\|6511 | 48 | 65 | 69 | 182 | | | | | |
| SLC25A34\|284723 | 51 | 50 | 55 | 156 | | | | | |
| SLC45A3\|85414 | 63 | 54 | 71 | 188 | | | | | |
| SLC5A9\|200010 | 47 | 39 | 53 | 139 | | | | | |
| SLC6A2\|6530 | 56 | 69 | 71 | 196 | | | | | |
| SLC7A9\|11136 | 58 | 71 | 80 | 209 | | | | | |
| SMAD6\|4091 | 54 | 62 | 61 | 177 | | | | | |
| SP140L\|93349 | 65 | 70 | 79 | 214 | | | | | |
| SPDYA\|245711 | 48 | 49 | 66 | 163 | | | | | |
| SPG7\|6687 | 57 | 70 | 73 | 200 | | | | | |
| SPOCD1\|90853 | 43 | 44 | 52 | 139 | | | | | |
| SPSB3\|90864 | 56 | 75 | 80 | 211 | | | | | |
| STAG3L3\|442578 | 53 | 60 | 73 | 186 | | | | | |
| STAP2\|55620 | 51 | 69 | 79 | 199 | | | | | |
| STAT6\|6778 | 56 | 47 | 52 | 155 | | | | | |
| SYCP3\|50511 | 54 | 54 | 57 | 165 | | | | | |
| TAF1C\|9013 | 56 | 69 | 74 | 199 | | | | | |
| TBC1D3\|729873 | 50 | 64 | 72 | 186 | | | | | |
| TBC1D3B\|414059 | 52 | 66 | 71 | 189 | | | | | |
| TBC1D3P2\|440452 | 54 | 68 | 84 | 206 | | | | | |
| TCEANC\|170082 | 52 | 50 | 50 | 152 | | | | | |
| TCTE3\|6991 | 60 | 72 | 87 | 219 | | | | | |
| TECR\|9524 | 48 | 65 | 69 | 182 | | | | | |
| THUMPD2\|80745 | 45 | 48 | 64 | 157 | | | | | |
| TIA1\|7072 | 44 | 52 | 68 | 164 | | | | | |
| TMC7\|79905 | 51 | 69 | 75 | 195 | | | | | |
| TMEM51\|55092 | 51 | 50 | 55 | 156 | | | | | |
| TNFAIP2\|7127 | 57 | 72 | 67 | 196 | | | | | |
| TNK1\|8711 | 72 | 85 | 88 | 245 | | | | | |
| TOP3B\|8940 | 65 | 77 | 77 | 219 | | | | | |
| TRAPPC2\|6399 | 52 | 50 | 50 | 152 | | | | | |
| TRIM26\|7726 | 51 | 61 | 70 | 182 | | | | | |
| TRIM27\|5987 | 53 | 63 | 70 | 186 | | | | | |
| TRIM38\|10475 | 55 | 65 | 73 | 193 | | | | | |
| TRPV1\|7442 | 73 | 85 | 89 | 247 | | | | | |
| TSPAN14\|81619 | 54 | 59 | 71 | 184 | | | | | |
| TSPYL6\|388951 | 45 | 50 | 66 | 161 | | | | | |
| TTLL3\|26140 | 65 | 73 | 90 | 228 | | | | | |
| UBD\|10537 | 51 | 61 | 71 | 183 | | | | | |
| UCP3\|7352 | 58 | 60 | 70 | 188 | | | | | |
| UNC93B1\|81622 | 57 | 60 | 69 | 186 | | | | | |
| UPK3A\|7380 | 67 | 77 | 81 | 225 | | | | | |
| U5F1\|7391 | 77 | 66 | 86 | 229 | | | | | |
| WASH3P\|374666 | 58 | 68 | 67 | 193 | | | | | |
| WDR52\|55779 | 55 | 71 | 80 | 206 | | | | | |
| WDR6\|11180 | 62 | 72 | 84 | 218 | | | | | |
| YTHDC1\|91746 | 50 | 55 | 68 | 173 | | | | | |
| ZCWPW1\|55063 | 53 | 60 | 69 | 182 | | | | | |
| ZFPM1\|161882 | 57 | 69 | 73 | 199 | | | | | |
| ZNF100\|163227 | 47 | 63 | 75 | 185 | | | | | |
| ZNF160\|90338 | 59 | 74 | 83 | 216 | | | | | |
| ZNF165\|1718 | 54 | 63 | 71 | 188 | | | | | |
| ZNF169\|169841 | 74 | 86 | 73 | 233 | | | | | |
| ZNF182\|7569 | 48 | 52 | 51 | 151 | | | | | |
| ZNF193\|7746 | 54 | 64 | 71 | 189 | | | | | |
| ZNF195\|7748 | 74 | 73 | 92 | 239 | | | | | |
| ZNF254\|9534 | 56 | 69 | 80 | 205 | | | | | |
| ZNF443\|10224 | 50 | 67 | 72 | 189 | | | | | |
| ZNF480\|147657 | 58 | 73 | 85 | 216 | | | | | |
| ZNF493\|284443 | 47 | 66 | 75 | 188 | | | | | |
| ZNF506\|440515 | 46 | 66 | 73 | 185 | | | | | |
| ZNF513\|130557 | 48 | 49 | 68 | 165 | | | | | |
| ZNF524\|147807 | 61 | 73 | 83 | 217 | | | | | |
| ZNF564\|163050 | 50 | 67 | 73 | 190 | | | | | |
| ZNF577\|84765 | 58 | 73 | 85 | 216 | | | | | |
| ZNF600\|162966 | 59 | 73 | 85 | 217 | | | | | |
| ZNF630\|57232 | 48 | 52 | 51 | 151 | | | | | |
| ZNF638\|27332 | 43 | 52 | 67 | 162 | | | | | |
| ZNF705A\|440077 | 60 | 60 | 61 | 181 | | | | | |
| ZNF708\|7562 | 46 | 66 | 75 | 187 | | | | | |
| ZNF763\|2S4390 | 49 | 68 | 73 | 190 | | | | | |
| ZNF799\|90576 | 50 | 67 | 72 | 189 | | | | | |
| ZMF814\|730051 | 62 | 74 | 82 | 218 | | | | | |
| ZNF823\|55552 | 48 | 69 | 73 | 190 | | | | | |
| ZNF841\|284371 | 58 | 73 | 85 | 216 | | | | | |
| ZNRD1\|30834 | 51 | 61 | 70 | 182 | | | | | |
| ZRANB2\|9406 | 45 | 38 | 55 | 138 | | | | | |
| ZRSR2\|8233 | 52 | 50 | 50 | 152 | | | | | |
| ZSCAN16\|80345 | 54 | 63 | 71 | 188 | | | | | |
| ZSCAN3B\|342933 | 61 | 72 | 82 | 215 | | | | | |
| C17orf86\|654434 | 0 | 0 | 0 | 0 | | | | | |
| C1orf126\|200197 | 0 | 0 | 0 | 0 | | | | | |
| CARD9\|64170 | 0 | 0 | 0 | 0 | | | | | |
| CHKB-CPT1B\|386593 | 0 | 0 | 0 | 0 | | | | | |
| CROCCL1\|84809 | 0 | 0 | 0 | 0 | | | | | |
| CYorf15B\|84663 | 0 | 0 | 0 | 0 | | | | | |
| FLJ12825\|440101 | 0 | 0 | 0 | 0 | | | | | |
| HCG26\|352961 | 0 | 0 | 0 | 0 | | | | | |
| HCG4P6\|80868 | 0 | 0 | 0 | 0 | | | | | |
| HLA-L\|3139 | 0 | 0 | 0 | 0 | | | | | |
| ID2B\|84099 | 0 | 0 | 0 | 0 | | | | | |
| LOC100144604\|100144604 | 0 | 0 | 0 | 0 | | | | | |
| LOC100272146\|100272146 | 0 | 0 | 0 | 0 | | | | | |
| LOC100286793\|100286793 | 0 | 0 | 0 | 0 | | | | | |
| LOC100288778\|100288778 | 0 | 0 | 0 | 0 | | | | | |
| LOC146880\|146880 | 0 | 0 | 0 | 0 | | | | | |
| LOC283314\|283314 | 0 | 0 | 0 | 0 | | | | | |
| LOC284232\|284232 | 0 | 0 | 0 | 0 | | | | | |
| LOC284233\|284233 | 0 | 0 | 0 | 0 | | | | | |
| LOC284900\|284900 | 0 | 0 | 0 | 0 | | | | | |
| LOC285074\|285074 | 0 | 0 | 0 | 0 | | | | | |
| LOC285359\|285359 | 0 | 0 | 0 | 0 | | | | | |
| LOC388692\|388692 | 0 | 0 | 0 | 0 | | | | | |
| LOC391322\|391322 | 0 | 0 | 0 | 0 | | | | | |
| LOC400927\|400927 | 0 | 0 | 0 | 0 | | | | | |
| LOC401052\|401052 | 0 | 0 | 0 | 0 | | | | | |
| LOC440944\|440944 | 0 | 0 | 0 | 0 | | | | | |
| LOC642846\|642846 | 0 | 0 | 0 | 0 | | | | | |
| LOC91316\|91316 | 0 | 0 | 0 | 0 | | | | | |
| NCRNA00105\|80161 | 0 | 0 | 0 | 0 | | | | | |
| NPIPL3\|23117 | 0 | 0 | 0 | 0 | | | | | |
| PAR1\|145624 | 0 | 0 | 0 | 0 | | | | | |
| PDXDC2\|283970 | 0 | 0 | 0 | 0 | | | | | |
| WASH7P\|653635 | 0 | 0 | 0 | 0 | | | | | |
| ZNF137\|7696 | 0 | 0 | 0 | 0 | | | | | |
| ZNF187\|7741 | 0 | 0 | 0 | 0 | | | | | |

### Example 6

### Analysis of DNA methylation

Test Method:

By use of the “correlation between mRNA expression and DNA methylation” in the Broad GDAC Firehose, 933 DNA methylation probes were obtained for identification of 1078 key genes obtained in Example 2, and each of them was most negatively correlated with the expression of the corresponding gene. The beta values of these DNA methylation probes were then extracted from the “jhu-usc.edu_BLCA.Human-Methylation450” file of TCGA. Subsequently, a multi-variable regularized Cox regression (a LASSO-based regression method) was used to identify a set of optimal genes with low multicollinearity from the above 933 DNA methylation probes. A total of 23 DNA methylation genes were retained as active synergistic variables for this analysis (see Table 8), and they also showed statistically significant differences s in the corresponding single-variable Cox regression model (i.e., the adjusted p value<0.05).

In the foregoing LASSO-based regression analysis, the obtained DNA methylation data set was subject to 10 cross-validation to determine the optimal values of the regularization parameters. The regression analysis was performed by use of an R package “glmnet”.

Test Results:

The DNA methylation circumstances of 1078 key genes screened in Example 2 were analyzed, and some of the DNA methylation features could be used as biomarkers for bladder cancer prognosis.

First, 933 DNA methylation probes were obtained for 1078 key genes, and the DNA methylation features which were most associated with the expression of the corresponding genes were identified. Then, a LASSO regression-based, multi-variable regularized Cox regression method was used to screen out 23 important DNA methylation genes that were most responsible for these input survival data (see Table 8). All of the 23 selected genes showed statistically significant differences in the corresponding single-variable Cox regression models, while the p-value was adjusted to be <0.05. Among the 23 DNA methylation genes, it has been reported that genes associated with play important roles in bladder cancer, such as JAG1, CLIC3, IRF1, and POLB (for example, see Shi T P et al., J Urol 2008, 180 (1): 361-366).

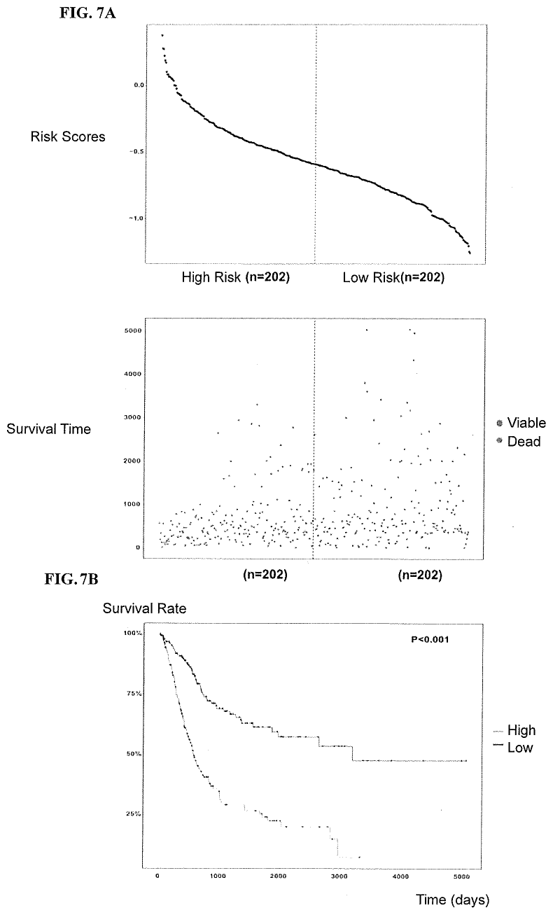

A risk value was then introduced, which was defined as the linear combination of the methylation levels (i.e., beta value) and the corresponding coefficients of the 23 DNA methylation genes in the regularized Cox regression. Next, all the BLCA patients were scored according to the median of the new risk value and divided into high-risk and low-risk groups. Kaplan-Meier analysis and log-rank test were then performed on these two groups of patients. The results showed that the high-risk group and the low-risk group showed significantly different risk score distributions (see FIG. 7A). In addition, it can be observed that the plotted Kaplan-Meier curve also has a significant difference, i.e., the higher the risk score, the worse the prognosis, and vice versa (see FIG. 7B). FIG. 7A shows the distribution of risk scores (based on the 23 selected DNA methylation genes) and the corresponding clinical features of patients in the high-risk and low-risk groups of DNA methylation analysis; the dotted line shows the cut-off value of the risk score. FIG. 7B shows Kaplan-Meier survival curves for the high-risk and low-risk groups, with statistical differences between the two groups by log-rank test. The results indicate that the new risk values based on the selected DNA methylation genes cars provide as good prognostic indicator for bladder cancer.

TABLE 8
23 Methylated Genes

| Gene Name | Correlation Coefficient |
|---|---|
| CYTH2 | −0.984161972 |
| PGLYRP4 | −0.835135351 |
| JAG1 | −0.758694541 |
| LTBP1 | −0.358058521 |
| CLIC3 | −0.344045267 |
| AKR1B1 | −0.21615728 |
| CNN3 | −0.174817703 |
| MESTIT1 | −0.165094565 |
| BAIAP2 | −0.091244951 |
| THBS3 | −0.078528329 |
| EIF2AK4 | −0.058860853 |
| KCNJ15 | 0.011163386 |
| MTERFD3 | 0.066920184 |
| PARP4 | 0.076173864 |
| IRF1 | 0.125102152 |
| TEAD4 | 0.247255028 |
| TIA1 | 0.293154238 |
| EFHD2 | 0.542824755 |
| PRRT4 | 0.641295163 |
| POLB | 0.703060414 |
| CRTC2 | 0.881500449 |
| C3orf19 | 1.083780825 |
| CCDC21 | 1.245618158 |

### Example7

### Analysis of Somatic Mutations

1078 key genes screened in Example 2 were analyzed for genomic features of the somatic mutations thereof.

Test Method:

After downloading the somatic mutation data from TCGA (Level 2), total 6052 somatic mutations in 908 genes were obtained from 1078 genes of 397 BLCA samples, wherein the 397 samples comprise 129 samples in Stage I/II, 135 samples in Stage III, and 133 samples in Stage IV.

Test Results:

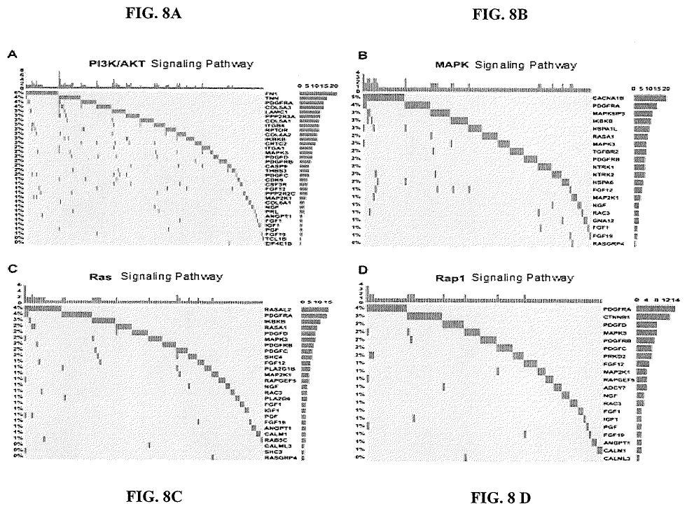

First, the pathways which might be affected by mutant genes were studied. Enrichment analysis of KEGG pathways of 908 mutant genes in the 1078 key genes was performed by DAVID (see Huang da W et al., Nat Protoc 2009, 4(1): 44-57), and it was found that a relatively large proportion of enrichment pathways had actually been considered as tumor-associated signaling pathways (see Table 9). In particular, there were four important pathways which had been proved to be associated with bladder cancer, that is, the PI3K/AKT pathway, the Ras pathway, the Rap1 pathway, and the MAPK pathway (see, for example, Houede N et al, Pharmacol Ther 2015, 145: 1-18). FIGS. 8A-8D show the significant enrichment of mutant genes for the PI3K-AKT pathway, the MAPK pathway, the Ras pathway, and the Rap1 pathway, respectively, in samples from BLCA patients. Of those, rows represent the mutant genes and are sequentially arranged in accordance with the frequency of the mutant genes in all samples; columns represent the involved samples (wherein the blank columns representing no mutation have been removed). The results of FIG. 8 show that a significant portion of the four pathways were mutated in bladder cancer. In particular, in all samples, 60% of the MAPK pathways, 56% of the PI3K/AKT pathways, 35% of the Rapl pathways, and 35% of the Ras pathways have had mutant genes, and the frequency of mutagenesis exceeds 1%. It can be observed that the four pathways have relatively high frequency of somatic mutations. This result is consistent with the previous studies that genetic mutations in important signaling pathways of cells are often driving tumorigenesis (see, e.g., Fawdar Set al., Proc Natl Acad Sci USA 2013, 110(30): 12426-12431).

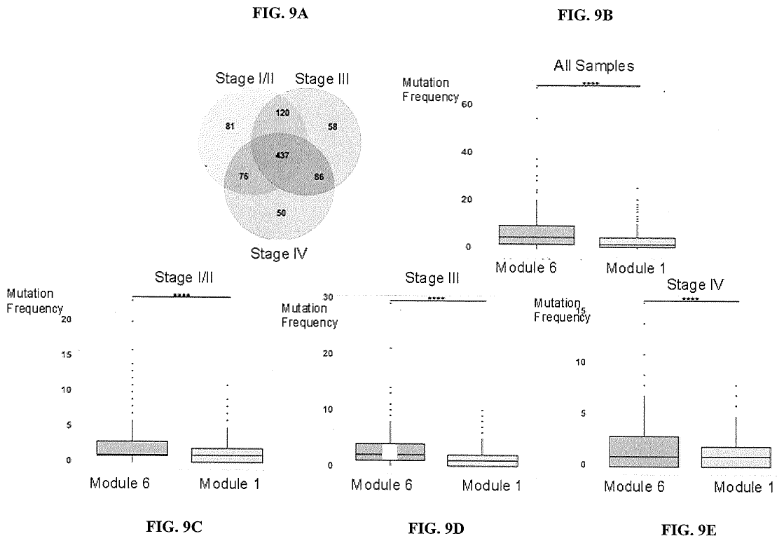

Meanwhile, the distribution of mutant genes in various bladder cancer stages were further analyzed (see FIG. 9). It was found that among the 1078 key genes, the BLCA patients in various stages shared most of the somatic mutant genes (437 genes) (see FIG. 9A). More importantly, it can be observed that the mutation frequency between the two modules (i.e., the corresponding blue and cyan modules in Example 4, which are most positively and negatively associated with different stages of tumor, respectively) has significant difference in samples of all or specific stages. In particular, the genes in the blue module (where all genes are risk effective genes) have more somatic mutations than the genes in the cyan module (93% of which are protective effective genes) (see FIGS. 9B-9E). This result indicates that even if somatic mutations are present in most of key genes, they are significantly biased toward the genes of the genome that are specifically associated with the tumor stage. This result provides useful clues for understanding of the effects of somatic mutations on various stages (progression) of bladder cancer.

TABLE 9
Results of KEG Analysis

| | | | Gene | | |
|---|---|---|---|---|---|
| Type | Term | Number | Ratio | P Value | Genes |
| KEGG_PATHWAY | hsa04014: | 26 | 2.872928 | 6.80E−05 | 801, 56034, 53358, 3479, 284, 80310, 9771, 2246, |
| | Ras signaling | | | | 4803, 5228, 5159, 8398, 5881, 5156, 5604, 3551, |
| | pathway | | | | 399694, 5921, 810, 9462, 2257, 5595, 115727, 5878, |
| | | | | | 9965, 5319 |
| KEGG_PATHWAY | hsa04151: | 34 | 3.756906 | 9.13E−05 | 842, 1291, 200186, 56034, 63923, 2335, 5617, 3479, |
| | PI3K-Akt | | | | 253314, 284, 80310, 2246, 4803, 3696, 1289, 5228, |
| | signaling | | | | 5159, 3672, 1441, 5156, 1284, 5604, 3551, 57521, |
| | pathway | | | | 5523, 1021, 5522, 3915, 2257, 50509, 5595, 9623, |
| | | | | | 7059, 9965 |
| KEGG_PATHWAY | hsa04721: | 12 | 1.325967 | 1.65E−04 | 161, 6812, 163, 523, 9114, 535, 26052, 774, 1213, |
| | Synaptic vesicle | | | | 245973, 526, 525 |
| | cycle | | | | |
| KEGG_PATHWAY | hsa04974: | 14 | 1.546961 | 2.48E−04 | 5222, 1291, 4225, 1284, 1299, 476, 6510, 643834, |
| | Protein | | | | 23436, 50509, 1289, 11136, 80781, 1506 |
| | digestion and | | | | |
| | absorption | | | | |
| KEGG_PATHWAY | hsa04510: | 23 | 2.541436 | 3.09E−04 | 824, 1291, 3672, 1284, 5881, 5156, 5604, 56034, |
| | Focal adhesion | | | | 2335, 63923, 3479, 53358, 399694, 1499, 80310, |
| | | | | | 3915, 50509, 3696, 7059, 5595, 1289, 5228, 5159 |
| KEGG_PATHWAY | hsa05218: | 11 | 1.21547 | 0.001848 | 80310, 1021, 2246, 5156, 2257, 5604, 5595, 56034, |
| | Melanoma | | | | 3479, 9965, 5159 |
| KEGG_PATHWAY | hsa05214: | 10 | 1.104972 | 0.003478 | 1021, 5156, 801, 5604, 5595, 53358, 3479, 399694, |
| | Glioma | | | | 810, 5159 |
| KEGG_PATHWAY | hsa04015: Rap1 | 20 | 2.209945 | 0.005327 | 5881, 113, 5156, 801, 5604, 56034, 3479, 284, 810, |
| | signaling | | | | 1499, 9771, 80310, 25865, 2246, 4803, 2257, 5595, |
| | pathway | | | | 5228, 9965, 5159 |
| KEGG_PATHWAY | hsa04966: | 6 | 0.662983 | 0.008164 | 523, 9114, 535, 245973, 526, 525 |
| | Collecting duct | | | | |
| | acid secretion | | | | |
| KEGG_PATHWAY | hsa04540: Gap | 11 | 1.21547 | 0.008838 | 80310, 2697, 5156, 113, 7846, 5604, 5595, 2983, |
| | junction | | | | 56034, 79861, 5159 |
| KEGG_PATHWAY | hsa05215: | 11 | 1.21547 | 0.008838 | 80310, 842, 5156, 5604, 5595, 56034, 3551, 3479, |
| | Prostate cancer | | | | 3645, 1499, 5159 |
| KEGG_PATHWAY | hsa04270: | 13 | 1.436464 | 0.011038 | 8398, 146, 147, 113, 5604, 801, 2768, 157855, 810, |
| | Vascular | | | | 1909, 2983, 5595, 5319 |
| | smooth muscle | | | | |
| | contraction | | | | |
| KEGG_PATHWAY | hsa04022: | 16 | 1.767956 | 0.012738 | 146, 147, 113, 5604, 801, 2768, 476, 157855, 7417, |
| | cGMP-PKG | | | | 8654, 810, 1909, 493, 151, 2983, 5595 |
| | signaling | | | | |
| | pathway | | | | |
| KEGG_PATHWAY | hsa04750: | 11 | 1.21547 | 0.018061 | 8398, 4803, 113, 41, 40, 801, 7442, 3479, 4914, |
| | Inflammatory | | | | 51393, 810 |
| | mediator | | | | |
| | regulation of | | | | |
| | TRP channels | | | | |
| KEGG_PATHWAY | hsa04512: | 10 | 1.104972 | 0.022394 | 1291, 3672, 1284, 3915, 50509, 3696, 7059, 2335, |
| | ECM-receptor | | | | 63923, 1289 |
| | interaction | | | | |
| KEGG_PATHWAY | hsa04810: | 18 | 1.98895 | 0.023632 | 3672, 5881, 5156, 5604, 56034, 2768, 2335, 80310, |
| | Regulation of | | | | 2246, 10458, 8826, 2257, 3696, 5595, 5962, 3645, |
| | actin | | | | 9965, 5159 |
| | cytoskeleton | | | | |
| KEGG_PATHWAY | hsa05230: | 8 | 0.883978 | 0.031963 | 6510, 2539, 5156, 5604, 5595, 4914, 5213, 5159 |
| | Central carbon | | | | |
| | metabolism in | | | | |
| | cancer | | | | |
| KEGG_PATHWAY | hsa04010: | 20 | 2.209945 | 0.035369 | 3305, 5881, 5156, 774, 5604, 2768, 3551, 23162, |
| | MARK | | | | 5921, 2246, 4803, 2257, 5595, 7048, 115727, 3310, |
| | signaling | | | | 4915, 4914, 9965, 5159 |
| | pathway | | | | |
| KEGG_PATHWAY | hsa04320: | 5 | 0.552486 | 0.037612 | 440822, 4854, 5604, 5595, 51513 |
| | Dorso-ventral | | | | |
| | axis formation | | | | |
| KEGG_PATHWAY | hsa04144: | 20 | 2.209945 | 0.039153 | 3305, 9265, 9266, 5156, 26052, 22905, 161, 3949, |
| | Endocytosis | | | | 163, 3482, 1213, 6643, 2359, 92421, 7048, 3310, |
| | | | | | 2869, 5878, 4914, 56904 |
| KEGG_PATHWAY | hsa05110: | 7 | 0.773481 | 0.039375 | 523, 9114, 535, 6558, 245973, 526, 525 |
| | Vibrio cholerae | | | | |
| | infection | | | | |

### Example 8

### Dynamic Change of MicroRNA Regulatory Network in Various Tumor Stages

The miRNA regulatory network of the key genes of various bladder cancer stages screened in Example 2 was analyzed for its dynamic change.

Test Method:

Network Analysis of MicroRNAs Regulatory Network

A R package “igraph” was used to calculate the synergic degree of microRNA regulatory network in various bladder cancer stages. The network plot was generated by Cytoscape 3.5.0.

Process of MicroRNA-mRNA Interaction Data

First, the interactions between microRNAs and the 1078 key genes screened form the miRWalk2.0 database which had been validated by experiments were obtained (see, Dweep Het al., Nat Methods 2015, 12 (8): 697). Then, the correlation coefficients between the expression values of 1078 key genes and the corresponding interaction microRNAs were calculated for each bladder cancer stages. If the correlation coefficient of a pair of microRNA and gene was less than −0.3, they were considered as a potential regulatory pair. Otherwise, the pair of microRNA and gene was removed from the initial microRNA-gene interacting network. Furthermore, specific microRNAs which are correlated with the bladder cancer can be found from the miRCancer database (December 2016 Edition) (see, Xie B et al., Bioinformatics 2013, 29 (5): 638-644).

Test Results:

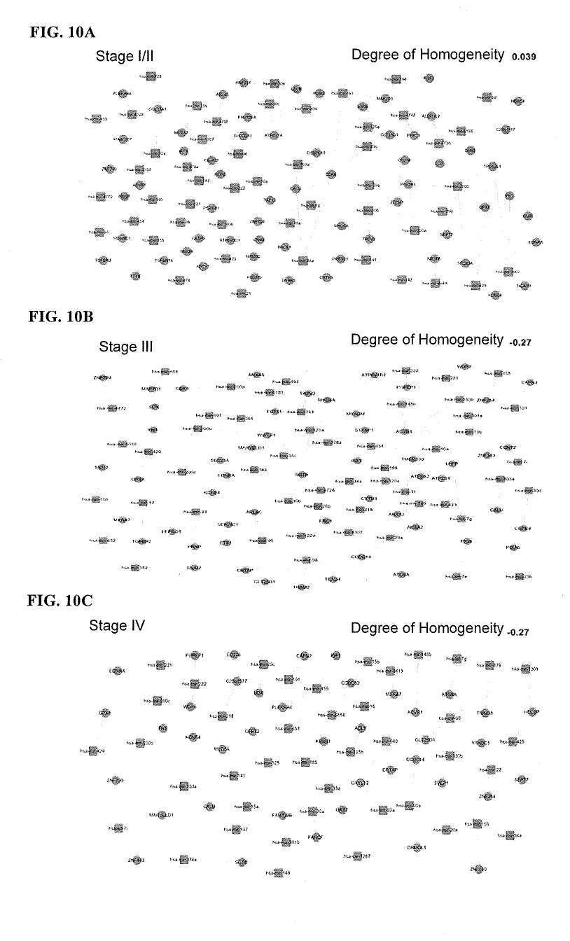

The microRNAs interacting with the 1078 key genes screened in Example 2 are shown in Table 10. By calculation of the correlation coefficients between the microRNAs and the expression values of the corresponding target genes were calculated, only the microRNA-gene pairs having a coefficient below −0.3 were selected as potential regulatory partners, and on the basis a microRNA-gene interacting network was constructed for each bladder cancer stage. It is found that in different bladder cancer stages (progression), the structure of the microRNA regulatory network (including the interactions involving microRNAs which are known to be BLCA-specific) tend to become sparser, and thus it can be seen that the interaction with each other is gradually reduced (see, FIG. 10). To quantify this trend, the synergic degrees of the individual microRNA networks in different stages were further calculated, and a significant decreasing trend was observed in Stage I/II, Stage III, and Stage IV: 0.039, −0.27 and −0.27. FIG. 10A-10C show the visual dynamic changes of the microRNA regulatory network in Stage I/II, Stage III, and Stage IV, respectively. Of those, the rectangles represent the selected microRNAs, and the known BLCA-specific microRNAs arc shown in red; and the target genes corresponding to the microRNAs arc represented by green circles, and the cooperation degrees of the individual networks arc also shown.

It can be seen that the microRNA regulatory network of 1078 genes screened from the BLCA patients showed a discretely increasing trend with the progression of bladder cancer, which is likely to be associated with the dysregulation of microRNAs in cancer cells. It also reflects the disorders of intracellular regulation and control gene expression in bladder cancer.

TABLE10
MicmRNAs Interacting with 1078 Key Genes

| Stage I + Stage II | | | Stage III | | | Stage IV | | |
|---|---|---|---|---|---|---|---|---|
| Label | Source | Target | Label | Source | Target | Label | Source | Target |
| miRCancer | hsa-mir-103a | CALCU | miRCancer | hsa-mir-101 | CAPN2 | miRCancer | hsa-mir-101 | CAPN2 |
| miRCancer | hsa-mir-103a | MYO5A | miRCancer | hsa-mir-103a | CALU | miRCancer | hsa-mir-103a | CALU |
| miRCancer | hsa-mir-103a | SLCO3A1 | miRCancer | hsa-mir-141 | FUTI1 | miRCancer | hsa-mir-103a | MYO5A |
| miRCancer | hsa-mir-141 | MYO5A | miRCancer | hsa-mir-141 | MYO5A | miRCancer | hsa-mir-125b | ACLY |
| miRCancer | hsa-mir-141 | PRSS23 | miRCancer | hsa-mir-141 | TRPV2 | miRCancer | hsa-mir-125b | KPNB1 |
| miRCancer | hsa-mir-141 | 7-Sep | miRCancer | hsa-mir-155 | ZNF254 | miRCancer | hsa-mir-141 | MYO5A |
| miRCancer | hsa-mir-141 | TRPV2 | miRCancer | hsa-mir-17 | LEPROT | miRCancer | hsa-mir-155 | ZNF160 |
| miRCancer | hsa-mir-155 | TSPAN14 | miRCancer | hsa-mir-17 | TGFBR2 | miRCancer | hsa-mir-155 | ZNF254 |
| miRCancer | hsa-mir-183 | C6orf72 | miRCancer | hsa-mir-182 | SNAI2 | miRCancer | hsa-mir-16 | CCDC80 |
| miRCancer | hsa-mir-186 | FGF1 | miRCancer | hsa-mir-183 | SGTB | miRCancer | hsa-mir-185 | GXYLT2 |
| miRCancer | hsa-mir-186 | MAP7D1 | miRCancer | hsa-mir-186 | ACVR1 | miRCancer | hsa-mir-200b | FN1 |
| miRCancer | hsa-mir-200a | CDK6 | miRCancer | hsa-mir-200a | CDK6 | miRCancer | hsa-mir-200b | GPX8 |
| miRCancer | hsa-mir-200a | 7-Sep | miRCancer | hsa-mir-200a | FUT11 | miRCancer | hsa-mir-200c | EDNRA |
| miRCancer | hsa-mir-200a | WWTR1 | miRCancer | hsa-mir-200a | WWTR1 | miRCancer | hsa-mir-200c | FN1 |
| miRCancer | hsa-mir-200b | FN1 | miRCancer | hsa-mir-200b | FN1 | miRCancer | hsa-mir-200c | GPX8 |
| miRCancer | hsa-mir-200b | GPX8 | miRCancer | hsa-mir-200b | GPX8 | miRCancer | hsa-mir-200c | KCNE4 |
| miRCancer | hsa-mir-200b | LOX | miRCancer | hsa-mir-200b | LOX | miRCancer | hsa-mir-218 | C20orf177 |
| miRCancer | hsa-mir-200b | SEC23A | miRCancer | hsa-mir-200b | SEC23A | miRCancer | hsa-mir-221 | PGPEP1 |
| miRCancer | hsa-mir-200b | 7-Sep | miRCancer | hsa-mir-200b | WWTR1 | miRCancer | hsa-mir-222 | PGPEP1 |
| miRCancer | hsa-mir-200b | TPD52L1 | miRCancer | hsa-mir-200c | EDNRA | miRCancer | hsa-mir-222 | WDR6 |
| miRCancer | hsa-mir-200b | WWTR1 | miRCancer | hsa-mir-200c | FN1 | miRCancer | hsa-mir-29c | CD276 |
| miRCancer | hsa-mir-200c | CNIH | miRCancer | hsa-mir-200c | GPX8 | miRCancer | hsa-mir-29c | LOX |
| miRCancer | hsa-mir-200c | EDNRA | miRCancer | hsa-mir-200c | KCNE4 | miRCancer | hsa-mir-34a | 7-Sep |
| miRCancer | hsa-mir-200c | FN1 | miRCancer | hsa-mir-200c | SEC23A | miRCancer | hsa-mir-429 | GPX8 |
| miRCancer | hsa-mir-200e | GPX8 | miRCancer | hsa-mir-218 | ANXA2 | miRCancer | hsa-mir-92a | GXYLT2 |
| miRCancer | hsa-mir-200c | KCNE4 | miRCancer | hsa-mir-221 | PGPEP1 | miRCancer | hsa-mir-99a | CCDC14 |
| miRCancer | hsa-mir-200c | NCAM1 | miRCancer | hsa-mir-222 | PGPEP1 | miRCancer | hsa-mir-99a | ORMDL1 |
| miRCancer | hsa-mir-200c | SEC23A | miRCancer | hsa-mir-222 | WDR6 | non-miRCancer | hsa-let-7g | ATG9A |
| miRCancer | hsa-mir-200c | 7-Sep | miRCancer | hsa-mir-23b | PDIA6 | non-miRCancer | hsa-let-7i | ZNF443 |
| miRCancer | hsa-mir-200c | TPD52L1 | miRCancer | hsa-mir-26b | ERC1 | non-miRCancer | hsa-let-7i | ZNF799 |
| miRCancer | hsa-mir-205 | TRPV2 | miRCancer | hsa-mir-34a | CYTH3 | non-miRCancer | hsa-mir-107 | CALU |
| miRCancer | hsa-mir-221 | PGPEP1 | miRCancer | hsa-mir-429 | GPX8 | non-miRCancer | hsa-mir-128 | FAM129B |
| miRCancer | hsa-mir-222 | PGPEP1 | miRCancer | hsa-mir-429 | SEC23A | non-miRCancer | hsa-mir-128 | GAS7 |
| miRCancer | hsa-mir-222 | WDR6 | miRCancer | hsa-mir-96 | ARL4C | non-miRCancer | hsa-mir-128 | GFPT2 |
| miRCancer | hsa-mir-222 | ZNF708 | miRCancer | hsa-mir-96 | SNAI2 | non-miRCancer | hsa-mir-1287 | GAS7 |
| miRCancer | hsa-mir-223 | PLEKHA6 | non-miRCancer | hsa-let-7e | PIGS | non-miRCancer | hsa-mir-1301 | HDLBP |
| miRCancer | hsa-mir-34a | CALU | non-miRCancer | hsa-let-7g | ATG9A | non-miRCancer | hsa-mir-130b | SVEP1 |
| miRCancer | hsiHnir-34a | CDK6 | non-miRCancer | bsa-let-7g | CALU | non-miRCancer | hsa-mir-148b | ACVRI |
| miRCancer | hsu-nur-34a | CYTH3 | non-miRCancer | hsa-let-7g | LHFP | non-miRCancer | hsa-mir-149 | SGTB |
| miRCancer | hsa-mtr-34a | DYRK3 | non-miRCancer | hsa-lel-7i | CCNT2 | non-miRCancer | hsa-mir-15a | CALU |
| miRCancer | hsa-mir-34a | MTMR2 | non-miRCancer | hsa-let-7i | ZNF443 | non-miRCancer | hsa-mir-15b | CCDC80 |
| miRCancer | hsa-mir-429 | GPX8 | non-miRCancer | hsa-mir-10a | TMEM109 | non-miRCancer | hsa-mir-15b | MXRA7 |
| miRCancer | hsa-mir-429 | SEC23A | non-miRCancer | hsa-mir-1229 | ERC1 | non-miRCancer | hsa-mir-20a | SVEP1 |
| miRCancer | hsa-mir-92a | COL18A1 | non-niiRCancer | hsa-mir-125a | ATP6V1B2 | non-miRCancer | hsa-mir-22 | YTHDC1 |
| miRCancer | hsa-mir-96 | C6orf72 | non-miRCa ncer | hsa-mir-125a | SGTB | non-miRCancer | hsa-mir-30a | FANCF |
| miRCancer | hsa-mir-2c | CD276 | non-miRCa ncer | hsa-mir-1307 | ERC1 | non-miRCancer | hsa-mir-33 a | GXYLT2 |
| miRCancer | hsa-mir-29c | CDK6 | non-miRCa ncer | hsa-mir-130b | ACVR1 | non-miRCancer | hsa-mir-3613 | MXRA7 |
| miRCancer | hsa-mir-29c | WWTR1 | non-miRCa ncer | hsa-mir-148b | ACVR1 | non-miRCancer | hsa-mir-378a | MARVELD1 |
| non-miRCancer | hsa-mir-29a | ZFPM1 | non-miRCa ncer | hsa-mir-15b | MXRA7 | non-miRCancer | hsa-mir-378a | SGTB |
| non-miRCancer | hsa-lct-7g | CALU | non-miRCancer | hsa-mir-15b | SIDT2 | non-miRCancer | hsa-mir-3913 | SGTB |
| non-miRCancer | hsa-mir-10a | CALU | non-miRCancer | hsa-mir-191 | CDK6 | non-miRCancer | hsa-mir-425 | HDLBP |
| non-miRCancer | hsa-mir-125a | GLT25D1 | non-miRCancer | hsa-mir-191 | MAP7D1 | non-miRCancer | hsa-mir-455 | IGF1 |
| non-miRCancer | hsa-mir-125a | SGTB | non-miRCancer | hsa-mir-197 | ACVR1 | non-miRCancer | hsa-mir-455 | PLEKHA6 |
| non-miRCancer | hsa-mir-1307 | C6orf72 | non-miRCancer | hsa-mir-19b | ACVR1 | non-miRCancer | hsa-mir-651 | CCDC80 |
| non-miRCancer | hsa-mir-1307 | IRF1 | non-miRCancer | hsa-mir-29a | AHSA2 | non-miRCancer | hsa-mir-6814 | CCDC80 |
| non-miRCancer | hsa-mir-142 | NR2F6 | non-miRCancer | hsa-mir-29a | CCDC14 | non-miRCancer | hsa-mir-876 | TRIM38 |
| non-miRCancer | hsa-mir-15a | BACE1 | non-miRCancer | hsa-mir-301a | ACVR1 | non-miRCancer | hsa-mir-940 | CRTAP |
| non-miRCancer | hsa-mir-15a | CALU | non-miRCancer | hsa-mir-30b | SGTB | non-miRCancer | hsa-mir-940 | GXYLT2 |
| non-miRCancer | hsa-mir-15a | CNN3 | non-miRCancer | hsa-mir-30c | SGTB | non-miRCancer | hsa-mir-98 | ATG9A |
| non-miRCancer | hsa-mir-15a | MYO5A | non-miRCancer | hsa-mir-30d | COPS8 | non-miRCancer | hsa-mir-98 | GLT25D1 |
| non-miRCancer | hsa-mir-15a | TAF13 | non-miRCancer | hsa-mir-31 | TMEM109 | | | |
| non-miRCancer | hsa-mir-15b | MXRA7 | non-miRCancer | bsa-mir-320a | CYTH3 | | | |
| norwniRCancer | hsa-mir-191 | CDK6 | non-miRCancer | hsa-mir-361 | WWTR1 | | | |
| non-miRCancer | hsa-mir-191 | MAP7D1 | non-miRCancer | hsa-mir-378a | IRF1 | | | |
| non-miRCancer | hsa-mir-193b | CASP9 | non-miRCancer | hsa-mir-378a | MARVELD1 | | | |
| non-miRCancer | hsa-mir-193b | PGPEP1 | non-miRCancer | hsa-mir-378a | MYADM | | | |
| non-miRCancer | hsa-mir-19b | ACVR1 | non-miRCancer | hsa-mir-378a | SGTB | | | |
| non-miRCancer | hsa-mir-21 | PDGFD | non-miRCancer | hsa-mir-378a | STXBP1 | | | |
| non-miRCancer | hsa-mir-22 | C20orf117 | non-miRCancer | hsa-mir-423 | ATP2B4 | | | |
| non-miRCancer | hsa-mir-22 | HDAC4 | non-miRCancer | hsa-mir-423 | ATP8B2 | | | |
| non-miRCancer | hsa-mir-29b | WWTR1 | non-miRCancer | hsa-mir-454 | ACVR1 | | | |
| non-miRCancer | hsa-mir-30c | ATP6V1A | non-miRCancer | hsa-mir-4728 | SGTB | | | |
| non-miRCancer | hsa-mir-30c | FAM126A | non-miRCancer | hsa-mir-4772 | ZNF799 | | | |
| non-miRCancer | hsa-mir-30c | LDLR | non-miRCancer | hsa-mir-484 | MAP7D1 | | | |
| non-miRCancer | hsa-mir-30c | OSBPL10 | non-miRCancer | hsa-mir-5010 | MAP7D1 | | | |
| non-miRCancer | hsa-mir-30c | WWTR1 | non-miRCancer | hsa-mir-652 | MXRA7 | | | |
| non-miRCancer | hsa-mir-30d | LDLR | non-miRCancer | hsa-mir-6781 | ANXA5 | | | |
| non-miRCancer | hsa-mir-30d | PGM3 | non-miRCancer | hsa-mir-769 | ANXA2 | | | |
| non-miRCancer | hsa-mir-30e | LDLR | non-miRCancer | hsa-mir-93 | ETF1 | | | |
| non-miRCancer | hsa-mir-30e | RNF217 | non-miRCancer | hsa-mir-93 | LEPROT | | | |
| non-miRCancer | hsa-mir-3199 | KIAA0907 | non-miRCancer | hsa-mir-93 | PRNP | | | |
| non-miRCancer | hsa-mir-378a | IRF1 | non-miRCancer | hsa-mir-93 | SER1NC1 | | | |
| non-miRCancer | hsa-mir-423 | ATP6V0D1 | non-miRCancer | hsa-mir-93 | SGTB | | | |
| non-miRCancer | hsa-mir-454 | ACVR1 | non-miRCancer | hsa-mir-98 | ATG9A | | | |
| non-miRCancer | hsa-mir-455 | PLEKHA6 | non-miRCancer | hsa-mir-98 | CRTAP | | | |
| non-miRCancer | hsa-mir-4649 | NR2F6 | non-miRCancer | hsa-mir-98 | GLT25D1 | | | |
| non-miRCancer | hsa-mir-4742 | PRR11 | non-miRCancer | hsa-mir-98 | TEAD4 | | | |
| non-miRCancer | hsa-mir-4756 | ARL4C | non-miRCancer | hsa-mir-98 | TRAM2 | | | |
| non-miRCancer | hsa-mir-4756 | MXRA7 | | | | | | |
| non-miRCancer | hsa-mir-4756 | SLCO3A1 | | | | | | |
| non-miRCancer | hsa-mir-4772 | ZNF799 | | | | | | |
| non-miRCancer | hsa-mir-5193 | DBN1 | | | | | | |
| non-miRCancer | hsa-mir-6728 | PLEKHA6 | | | | | | |
| non-miRCancer | hsa-mir-6730 | ALDH1L2 | | | | | | |
| non-miRCancer | hsa-mir-874 | ADCY7 | | | | | | |
| non-miRCancer | hsa-mir-93 | PRNP | | | | | | |
| non-miRCancer | hsa-mir-93 | SERINC1 | | | | | | |
| non-miRCancer | hsa-mir-93 | STYX | | | | | | |
| non-miRCancer | hsa-mir-93 | TGFBR2 | | | | | | |
| non-miRCancer | hsa-mir-98 | SNX24 | | | | | | |

### Example 9

### Comprehensive Analysis of Various Factors on Bladder Cancer Stages

For comprehensive understanding of various genomes and clinical factors on the bladder cancer progression, an ordered logistic regression model is used for comprehensive analysis of these factors.

Test Method

Ordered Logistic Regression for Comprehensive Analysis

The “mnrfit” function in Matlab 2016b was used to execute an ordinal logistic regression task. In this comprehensive analysis, the response variable was the tumor stage (stage IV=1, stage III=2, stage I/II=3), while the predictive variables included the mean expression values of protective effective genes and risk effective genes (z-normalized), the frequency of copy number variations (z-normalized), the risk scores of DNA methylation, the age and the gender (male=0, female=1).

Test Results

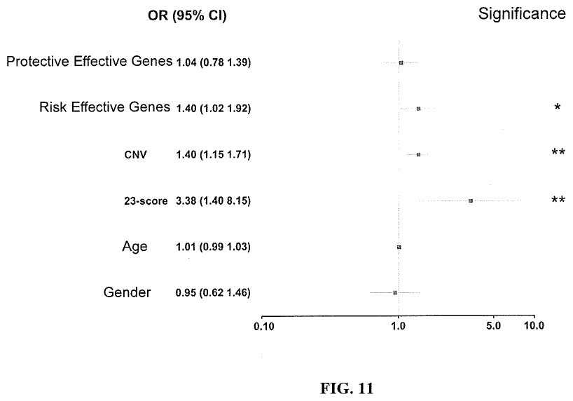

The mean expression (z-normalized) of the protective effective genes and the risk effective genes, the frequency of copy number variations (z-normalized), the risk scores of DNA methylation, the age and the gender were considered in the comprehensive analysis (see Table 11). As shown in the forest plot in FIG. 11, it can be observed that the mean expression of the risk genes, the frequency of copy number variations, and the risk scores of DNA methylation can significantly affect the stage of bladder cancer. In FIG. 11, the boxes and the lines represent the odds ratio (OR) and the corresponding 95% confidence interval, respectively, and the asterisks “*” represent statistically significant variables. Of those, *: p value<0.05; **: p value<0.01.

The ORs of these factors are all greater than 1, indicating that they can be considered as risk factors for bladder cancer progression. All the comprehensive modeling results are consistent with the results of the single-variable analyses in Examples 2-8. Thus, even the genomic data have heterogeneity as they come from different platforms, the multi-angle, multi-index comprehensive analysis and the clinical information thereof provides a reliable basis for study of the combined effect of bladder cancer genome as well as the clinical factors on tumor progression.

TABLE 11
Results of Comprehensive Analysis

| | B | stats. p | Parallel Tests |
|---|---|---|---|
| intercept1 | −0.6617 | 0.3585 | 0.089 |
| intercept2 | 0.9609 | 0.183 | |
| Average of protective effective genes | 0.0366 | 0.8041 | |
| Average of risk effective genes | 0.3386 | 0.0356 | |
| CNV | 0.3349 | 0.001 | |
| DNA | 1.2193 | 0.0066 | |
| Age | 0.0084 | 0.3649 | |
| Gender | −0.048 | 0.8246 | |

After Exp Transformation

| | |
|---|---|
| Average of protective effective genes | 1.037278027 (0.776467882 1.385415369) |
| Average of risk effective genes | 1.40298204 (1.02326654 1.923218337*) |
| CNV | 1.397800598 (1.145681894 1.705741647**) |
| DNA | 3.384817532 (1.404947591 8.149853894**) |
| Age | 1.008435379 (0.990049834 1.027367803) |
| Gender | 0.953133787 (0.623130071 1.457904309) |

The foregoing detailed description is provided for illustrative and exemplary purposes, and not intended to limit the scope of the accompanying claims. Various modifications of the embodiments as currently listed herein are obvious fat persons ordinarily skilled in the art, and fall within the scope of the accompanying claims and its equivalences.

1. A device of identifying a biological indicator of capable of evaluating a tumor progression comprising:
1) a clinical feature module capable of providing a clinical feature of a patient with said tumor, wherein said clinical feature comprises a tumor stage of said patient and/or a survival time of said patient;
2) a biological indicator module capable of providing at least one biological indicator derived from the patient;
3) a correlation determination module capable of determining a correlation between said at least one biological indicator of said individual patient with said clinical feature of the corresponding patient; and
4) an identification module capable of identifying said biological indicator which is determined to be correlated with said clinical feature in the module 3) as being capable of evaluating the tumor progression.

2. A device of identifying a biological indicator capable of evaluating a tumor progression comprising a computer for identifying said biological indicator, said computer is programmed to executing the steps of:
1) providing a clinical feature of a patient with said tumor, wherein said clinical feature comprises a tumor stage of said patient and/or a survival time of said patient;
2) providing at least one biological indicator derived from the patient;
3) determining a correlation between said at least one biological indicator of said individual patient and said clinical feature of the corresponding patient; and
4) identifying said biological indicator which is determined to be correlated with said clinical feature in 3) as being capable of evaluating said tumor progression.

3. A method of identifying a biological indicator capable of evaluating a tumor progression comprising:
1) providing a clinical feature of a patient with said tumor, wherein said clinical feature comprises a tumor stage of said patient and/or a survival time of said patient;
2) providing at least one biological indicator derived from said patient;
3) determining a correlation between said at least one biological indicator of said individual patient and said clinical feature of the corresponding patient; and
4) identifying said biological indicator which is determined to be correlated with said clinical feature in 3) as being capable of evaluating said tumor progression.

4. The device of claim 1, wherein said tumor comprises a bladder cancer.

5. The device of claim 1, wherein said at least one biological indicator comprises one or more classes of indicators selected from the group consisting of:
Class 1: an expression level of gene in said patient;
Class 2: a copy number variation of gene in said patient;
Class 3: a DNA methylation of gene in said patient;
Class 4: a somatic mutation of gene in said patient; and
Class 5: a microRNA in said patient.

6. The device of claim 5, wherein said at least one biological indicator comprises the expression level of gene in said patient, and determining a correlation between the expression level of said gene and said clinical feature comprises: performing a single variable regression analysis against said clinical feature by use of said expression level of said gene as the single variable, and identifying the genes of which a p value is less than or equal to a first threshold and a FDR value is less than or equal to a second threshold in the regression analysis as being correlated with said clinical feature.

7. The device of claim 5, wherein said at least one biological indicator comprises the expression level of said gene in said patient, and determining a correlation between the expression level of said gene and said clinical feature comprises performing a multiple-variable regression analysis against the clinical feature, and identifying the gene of which a FDR value is less than or equal to a third threshold in the regression analysis as being correlated with said clinical feature, and wherein the multiple variable comprises the expression level of said gene in said patient, the age of said patient, the gender of said patient, and/or the tumor stage of said patient.

8. The device of claim 5, wherein the at least one biological indicator comprises the expression level of gene in the patient, and the determining the correlation between the expression level of said gene and said clinical feature further comprises: determining the expression level of gene in the patient in an individual tumor stage, determining accordingly a co-expression circumstance of genes which is specific for tumor staging, classifying said genes into two or more groups in accordance with the co-expression circumstance of the genes, and determining the correlation between the expression level of gene of each group and said clinical feature.

9. A device of determining a tumor progression in a subject, comprising:
a) an analysis module capable of measuring an expression level of one or more genes as shown in Table 1 in said subject or a biological sample derived from said subject; and
b) a determination module capable of determining said tumor progression of said subject in accordance with the expression level as measuring in a).

10. A device of determining a tumor progression in a subject, comprising a computer for determining a tumor progression in a subject, said computer being programmed to executing the steps of:
a) determining the expression levels of one or more genes as shown in Table 1 in said subject or a biological sample derived from said subject; and
b) determining the tumor progression in the subject in accordance with the expression level as measured in a).

11. A method of determining a tumor progression in a subject, comprising:
a) measuring an expression level of one or more genes as shown in Table 1 in the subject or a biological sample derived from the subject; and
b) determining the tumor progression in the subject in accordance with the expression level as measured in a).

12. The device of claim 9, wherein the tumor progression comprises the tumor stage and/or a survival rate of the subject.

13. The device of claim 9, wherein the tumor comprises bladder cancer.

14. The device of claim 9, wherein the one or more genes comprises u least one or more genes as shown in Table 4.

15. The device of claim 9, wherein the one or more genes comprises tit least one or more genes as shown in Table 5.

16. The device of claim 9, wherein determining the expression levels of one or more genes as shown in Table 1 in the subject or a biological sample derived from the subject comprises: determining the average expression level of the genes as shown in Table 2 in said one or more genes; and determining the average expression level of the genes as shown in Table 3 in said one or more genes.

17. The device of claim 16, wherein determining the tumor progression in the subject is in accordance with Formula (I):
$$ln ( \frac{P ( Stages 1 )}{1 - P ( Stages 1 )} ) = Intercept + 0.0366 * a + 0.3386 * b + 0.3349 * c + 1.2193 * d + 0.0084 * e - 0.048 * f \qquad ( I )$$
wherein, when j=tumor stage III, Intercept=0.9609; and j=tumor stage I/II, Intercept=−0.6617;
a is the average expression level of the eerier as shown in Table 2 in the one or more genes;
b is the average expression level of the genes as shown in Table 3 in the one or more genes;
c is the copy number variation of the one or more genes;
d is the risk value of DNA methylation of the genes as shown in Table 8 in the one or more genes;
e is the subject's age; and
f is the subject's gender, wherein male is 0, and female is 1.

18. A device of treating a tumor in a subject comprising:
a) an analysis module capable of determining the expression levels of one or more genes as shown in Table 1 in the subject or a biological sample derived from the subject;
b) a determination module capable of determining the tumor progression in the subject in accordance with the expression level as measured in a); and
c) a treatment module capable of administering an effective amount of treatment to the subject in accordance with the progression as determined in b).
